(19) United States
(12) Patent Application Publication
(10) Pub. No.: US 2011/0224193 A1
(43) Pub. Date: Sep. 15, 2011

(54) INHIBITORS OF DIACYLGLYCEROL ACYLTRANSFERASE

(75) Inventors: Pauline C. Ting, New Providence, NJ (US); Robert Aslanian, Rockaway, NJ (US); Jianhua Cao, Edison, NJ (US); Mary Ann Caplen, Sayreville, NJ (US); Tin-Yau Chan, Edison, NJ (US); Hyunjin Kim, Livingston, NJ (US); Jae-Hun Kim, Scotch Plains, NJ (US); Rongze Kuang, Green Brook, NJ (US); Joe F. Lee, Brooklyn, NY (US); Heping Wu, Edison, NJ (US)

(73) Assignee: MERCK SHARP & DOHME CORP

(21) Appl. No.: 13/129,821

(22) PCT Filed: Nov. 17, 2009
(86) PCT No.: PCT/US09/64762

§ 371 Date: May 18, 2011

Related U.S. Application Data
(60) Provisional application No. 61/115,995, filed on Nov. 19, 2008.

(51) Int. Cl. A61K 31/5513; C07D 413/14; A61K 31/4545; A61K 31/497; A61P 3/00; A61P 3/04; A61P 3/10; A61P 9/00; A61P 3/08

(52) U.S. Cl. 514/218; 546/193; 514/318; 514/253.1; 544/364; 540/492

(57) ABSTRACT

The present invention relates to novel heterocyclic compounds as diacylglycerol acyltransferase (“DGAT”) inhibitors, pharmaceutical compositions comprising the heterocyclic compounds and the use of the compounds for treating or preventing a cardiovascular disease, a metabolic disorder, obesity or an obesity-related disorder, diabetes, dyslipidemia, a diabetic complication, impaired glucose tolerance or impaired fasting glucose. An illustrative compound of the invention is shown with this abstract.

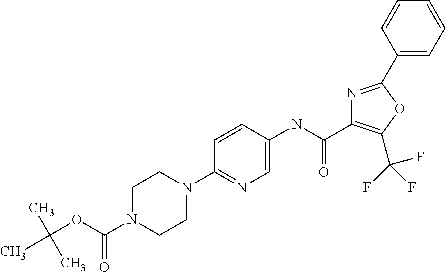

### FIELD OF THE INVENTION

The present invention relates to certain heterocyclic compounds useful as diacylglycerol acyltransferase (“DGAT”) inhibitors, especially diacylglycerol acyltransferase 1 (“DGAT1”) inhibitors, pharmaceutical compositions containing the compounds, and methods of treatment using the compounds and compositions to treat or prevent various diseases including cardiovascular disease, dyslipidemia, obesity and diabetes (e.g., Type 2 diabetes).

### BACKGROUND OF THE INVENTION

There is a need for additional ways of treating diseases associated with metabolic syndrome such as, for example, dyslipidemia, cardiovascular disease, obesity and diabetes (e.g., Type 2 diabetes).

Triglycerides or triacylglycerols are the major form of energy storage in eukaryotic organisms. In mammals, these compounds are primarily synthesized in three tissues: the small intestine, liver, and adipocytes. Triglycerides or triacylglycerols support the major functions of dietary fat absorption, packaging of newly synthesized fatty acids and storage in fat tissue (see Subauste and Burant, Current Drug Targets—Immune, Endocrine & Metabolic Disorders (2003) 3, pp. 263-270).

Diacylglycerol O-acyltransferase, also known as diglyceride acyltransferase or DGAT, is a key enzyme in triglyceride synthesis. DGAT catalyzes the final and rate-limiting step in the triacylglycerol synthesis from 1,2-diacylglycerol (DAG) and long chain fatty acyl CoA as substrates. Thus, DGAT plays an essential role in the metabolism of cellular diacylglycerol and is critically important for triglyceride production and energy storage homeostasis (see Mayorek at al. European Journal of Biochemistry (1989) 182, pp. 395-400).

Two forms of DGAT have been cloned and are designated DGAT1 and DGAT2 [see Cases et al, Proceedings of the National Academy of Science, USA (1998) 95, pp. 13018-13023, Lardizabal at al, Journal of Biological Chemistry (2001) 276, pp. 38862-38869 and Cases et al, Journal of Biological Chemistry (2001) 276, pp. 38870-38876]. Although both enzymes utilize the same substrates, there is no homology between DGAT1 and DGAT2. Both enzymes are widely expressed however some differences do exist in the relative abundance of expression in various tissues.

Disorders or imbalances in triglyceride metabolism, both absorption as well as de novo synthesis, have been implicated in the pathogenesis of a variety of disease risks. These include obesity, insulin resistance syndrome, Type II diabetes, dyslipidemia, metabolic syndrome (syndrome X) and coronary heart disease [see Kahn, Nature Genetics (2000) 25, pp. 6-7, Yanovski and Yanovski, New England Journal of Medicine (2002) 346, pp. 591-602, Lewis et al, Endocrine Reviews (2002) 23, pp. 201, Brazil, Nature Reviews Drug Discovery (2002) 1, pp. 408, Malloy and Kane, Advances in Internal Medicine (2001) 47, pp. 111, Subauste and Burant, Current Drug Targets—Immune, Endocrine & Metabolic Disorders (2003) 3, pp. 263-270 and Yu and Ginsberg, Annals of Medicine (2004) 36, pp. 252-261]. Compounds that can decrease the synthesis of triglycerides from diacylglycerol by inhibiting or lowering the activity of the DGAT enzyme would be of value as therapeutic agents for the treatment of diseases associated with abnormal metabolism of triglycerides.

Known inhibitors of DGAT include: dibenzoxazepinones (see Ramharack et al, EP1219716 and Burrows et al, 26th National Medicinal Chemistry Symposium (1998) poster C-22), substituted amino-pyrimidino-oxazines (see Fox et al, WO2004047755), chalcones such as xanthohumol (see Tabata et al, Phytochemistry (1997) 46, pp. 683-687 and Casaschi et al, Journal of Nutrition (2004) 134, pp. 1340-1346), substituted benzyl-phosphonates (see Kurogi et al. Journal of Medicinal Chemistry (1996) 39, pp. 1433-1437, Goto at al, Chemistry and Pharmaceutical Bulletin (1996) 44, pp. 547-551, Ikeda et al, Thirteenth International Symposium on Athersclerosis (2003), abstract 2P-0401, and Miyata et al, JP 2004067635), aryl alkyl acid derivatives (see Smith et al, WO2004100881 and US20040224997), furan and thiophene derivatives (see WO2004022551), pyrrolo[1,2b]pyridazine derivatives (see Fox at al, WO2005103907), and substituted sulfonamides (see Budd Haeberlein and Buckett, WO20050442500).

Also known to be inhibitors of DGAT are; 2-bromo-palmitic acid (see Colman et al, Biochimica et Biophysica Acta (1992) pp. 1125, 203-9), 2-bromo-octanoic acid (see Mayorek and Bar-Tana, Journal of Biological Chemistry (1985) 260, pp. 6528-6532), roselipins (see Noriko et al, (Journal of Antibiotics (1999) 52, pp. 815-826), amidepsin (see Tomoda et al, Journal of Antibiotics (1995) 48, pp. 42-7), isochromophilone, prenylflavonoids (see Chung et al, Planta Medica (2004) 70, v58-260), polyacetylenes (see Lee et al, Planta Medica (2004) 70, pp. 97-200), cochlioquinones (see Lee et al, Journal of Antibiotics (2003) 56, pp. 967-969), tanshinones (see Ko et al, Archives of Pharmaceutical Research (2002) 25, pp. 446-448), gemfibrozil (see Zhu et al, Atherosclerosis (2002) 164, pp. 221-228), and substituted quinolones (see Ko et al, Planta Medica (2002) 68, pp. 1131-1133). Also known to be modulators of DGAT activity are antisense oligonucleotides (see Monia and Graham, US20040185559).

Particular mention is made to PCT publication WO 2007/060140 (published May 31, 2007; applicant: F. Hoffmann-La Roche AG). Claim 1 therein discloses compounds of the formula:

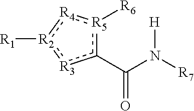

wherein R₁, R₂, R₃, R₄, R₅, R₆ and R₇ are described. Additional publications include WO 2008/141976 (published May 13, 2008), US 2009/0093497 (published May 1, 2009) and US 2009/0105273 (published May 1, 2009).

A need exists in the art, however, for additional DGAT inhibitors that have efficacy for the treatment of metabolic disorders such as, for example, obesity, Type it diabetes mellitus and metabolic syndrome.

### SUMMARY OF THE INVENTION

In an embodiment, this invention discloses a compound, or pharmaceutically acceptable salts, solvates, ester or prodrugs of said compound, or pharmaceutically acceptable salts, solvates or esters of said prodrug, the compound being represented by the general formula

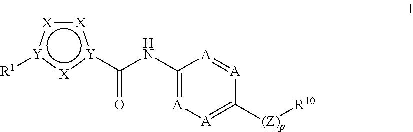

wherein:
each A is independently selected from C(R³) and N;

or alternately the moiety:

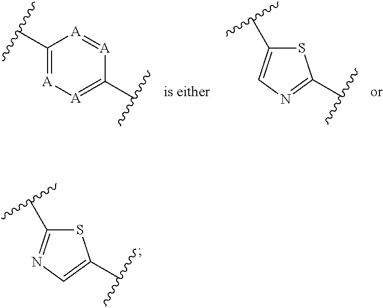

X is independently selected from C(R³), N, N(R⁴), O and S, provided that no more than one X is S or O, and at least one X or one Y is N, O, or S;
Y is independently selected from C and N;
Z is independently a bond, O or NR⁴;
p is 0 or 1;
R¹ is selected from aryl, heteroaryl, alkyl or cycloalkyl, wherein said aryl is unsubstituted or optionally independently substituted with one or more moieties which are the same or different, each substituent being independently selected from the group consisting of alkyl, haloalkoxy, methoxy-ethoxy alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR^{c}, —C(O)R^{c}, —C(O)OR^{c}, —C(O)N(R^{c})(R^{d}), —SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}), —N(R^{c})(R^{d}), -alkyl-N(R^{c})(R^{d}), —N(R^{c})C(O)R^{d}, —CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d}, —N(R^{c})S(O)₂N(R^{d})(R^{b}), —N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{c}, —N₃, —NO₂ and —S(O)₂R^{c}, wherein each R^{b}, R^{c} and R^{d}, is independently selected;
R³ is selected from the group of H, lower alkyl, hydroxy, halo, O-alkyl, O-haloalkyl, O-cycloalkyl, S-alkyl, S-haloalkyl, CN, CF₃, —SF₅, —OSF₅, —Si(R^{c})₃, —SR^{c}, cycloalkyl, heterocyclyl, haloalkyl, aryl, heteroaryl, N-alkyl, N-haloalkyl, NH₂, and N-cycloalkyl;
R⁴ is selected from the group of H, lower alkyl, cycloalkyl, heterocyclyl, haloalkyl, aryl, and heteroaryl;
R¹⁰ is either (i) a 4-8 membered heterocyclyl ring having from 1 to 3 ring N atoms, or (ii) a bicyclic heterocyclyl ring having from 1 to 3 ring N atoms,
wherein each of said heterocyclyl ring or bicyclic heterocyclyl ring for R¹⁰ is optionally fused with a heteroaryl ring, further wherein each of said heterocyclyl ring or bicyclic heterocyclyl ring for R¹⁰ is independently unsubstituted or optionally substituted, off of either (i) a ring N atom or (ii) a ring carbon atom on said heterocyclyl ring or said bicyclic heterocyclyl ring, with one or more G moieties wherein said G moieties can be the same or different, each G moiety being independently selected from the group consisting of:

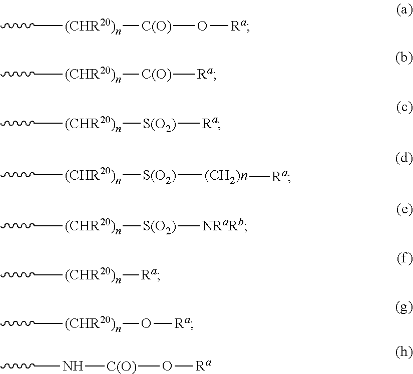

off of only C and not off of N, with the proviso that R¹⁰ is not a 5- or 6-membered heterocyclyl ring;

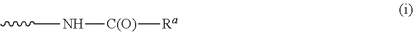

off of only C and not off of N, with the proviso that R¹⁰ is not a 5- or 6-membered heterocyclyl ring;

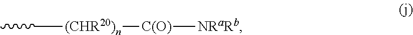

with the proviso that R¹⁰ is not a 5- or 6-membered heterocycyl ring;

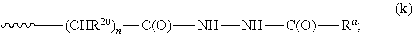

off of only C and not off of N;

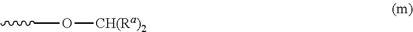

off on only C and not off of N; n) an oxo group off of only C and not off of N;

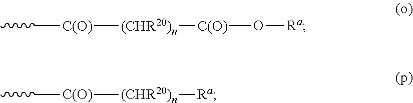

and (q) a spirocyclyl group;
wherein R^{a} is selected from the group consisting of hydrogen, hydroxy, CN, halo, alkyl, haloalkyl, alkenyl, alkynyl, aryl, heteroaryl, heterocyclyl, cycloalkyl or spirocyclyl, wherein each of said alkyl, alkenyl, alkynyl, aryl, heteroaryl, heterocyclyl and cycloalkyl is unsubstituted or optionally independently substituted with one or more moieties which are the same or different, each moiety being selected independently from the group consisting of O-haloalkyl, S-haloalkyl, CN, NO₂, CF₃, cycloalkyl, heterocyclyl, haloalkyl, aryl, heteroaryl, N-alkyl, N-haloalkyl, and N-cycloalkyl; alkyl, alkenyl, alkynyl, cycloalkylalkyl, cycloalkenyl, heterocyclylalkyl, aryl, arylalkyl, heteroaryl, heteroarylalkyl, halo, —OR^{c}, —C(O)R^{c}, —C(O)OR^{c}, —C(O)N(R^{c})(R^{d}), SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}), —N(R^{c})(R^{d}), -alkyl-N(R^{c})(R^{d}), —N(R^{c})C(O)R^{d}, —CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d}, —N(R^{c})S(O)₂N(R^{d})(R^{b}), —N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{c}, —N₃, and —S(O)₂R^{c}; and
wherein each R^{b}, R^{c} and R^{d} is independently selected;
R^{b} is H, lower alkyl, cycloalkyl, aryl, heteroaryl or heterocycloalkyl;
R^{c} is H, lower alkyl, cycloalkyl, aryl, heteroaryl or heterocycloalkyl;
R^{d} is H, lower alkyl, cycloalkyl, aryl, heteroaryl or heterocycloalkyl;
wherein each of said alkyl, cycloalkyl, aryl, heteroaryl or heterocycloalkyl in R^{b}, R^{c}, and R^{d} can be unsubstituted or optionally independently substituted with 1-2 substituents independently selected from halo, OH, NH₂, CF₃, CN, Oalkyl, NHalkyl, N(alkyl)₂ and Si(alkyl)₃;
   R²⁰ is H, —OH, halo, or —CF₃;
   m is 1-3, and
   n is 0-3.

The term “spirocyclyl” refers to a cyclic group substituted off the same carbon atom. A non-limiting example would be:

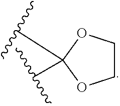

The term “oxo” refers to the moiety ═C(O) substituted off the same carbon atom.

The term “bicyclic heterocyclyl” refers to bicyclic compounds containing heteroatom as part of the ring atoms. A non-limiting example would be:

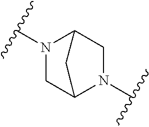

with no limitation as to the position of the heteroatom.

When a disubstituted moiety is shown with [symbol] on both sides, the attachment points are from left to right when looking at the parent formula, e.g. Formula I. Thus, for example, if the moiety:

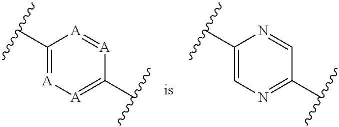

in Formula I, it means that the pyrazine ring is attached to NH on the left hand side and R¹⁰ on the right hand side in Formula I.

In another aspect, this invention provides compositions comprising at least one compound of Formula I.

In another aspect, this invention provides pharmaceutical compositions comprising at least one compound of Formula I and at least one pharmaceutically acceptable carrier.

In another aspect, this invention provides a method of treating diabetes in a patient in need of such treatment using therapeutically effective amounts of at least one compound of Formula I, or of a composition comprising at least one compound of Formula I.

In another aspect, this invention provides a method of treating diabetes in a patient in need of such treatment, e.g., Type 2 diabetes, using therapeutically effective amounts of at least one compound of Formula I, or of a composition comprising at least one compound of Formula I.

In another aspect, this invention provides a method of treating metabolic syndrome in a patient in need of such treatment, using therapeutically effective amounts of at least one compound of Formula I, or of a composition comprising at least one compound of Formula I.

In another aspect, this invention provides a method of inhibiting DGAT using therapeutically effective amounts of at least one compound of Formula I, or of a composition comprising at least one compound of Formula I.

In another aspect, this invention provides a method of inhibiting DGAT1 using therapeutically effective amounts of at least one compound of Formula I, or of a composition comprising at least one compound of Formula I.

### DESCRIPTION OF THE INVENTION

In an embodiment, the present invention discloses compounds of Formula I, or pharmaceutically acceptable salts, solvates, esters or prodrugs thereof.

The following embodiments (stated as “another embodiment”) are independent of each other; different such embodiments can be independently selected and combined in various combinations. Such combinations should be considered as part of the invention.

In another embodiment, A is C(R³).

In another embodiment, A is N.

In another embodiment, one A is N and the other A moieties are C(R³).

In another embodiment, one A is C(R³) and the other A moieties are N.

In another embodiment, two A moieties are N and the other two A moieties are C(R³).

In another embodiment, X is C(R³).

In another embodiment, X is N.

In another embodiment, X is N(R⁴).

In another embodiment, X is O.

In another embodiment, X is S.

In another embodiment, at least one X is O.

In another embodiment, at least one Y is N.

In another embodiment, one X is O and one other X is N.

In another embodiment, one X is O, one X is N and the other X is C(R³).

In another embodiment, Y is C.

In another embodiment, Y is N.

In another embodiment, R¹ is unsubstituted aryl.

In another embodiment. R¹ is aryl substituted as previously described.

In another embodiment, R¹ is unsubstituted heteroaryl.

In another embodiment, R¹ is heteroaryl substituted as previously described.

In another embodiment, R¹ is unsubstituted alkyl.

In another embodiment, R¹ is alkyl substituted as previously described.

In another embodiment, R¹ is unsubstituted cycloalkyl.

In another embodiment, R¹ is cycloalkyl substituted as previously described.

In another embodiment, R³ is H.

In another embodiment, R³ is lower alkyl.

In another embodiment, R³ is hydroxyl.

In another embodiment, R³ is —O-alkyl.

In another embodiment, R³ is —CN.

In another embodiment, R³ is —CF₃.

In another embodiment, R³ is —O— haloalkyl.

In another embodiment, R³ is —OSF₅.

In another embodiment. R³ is —SF₅.

In another embodiment, R⁴ is H.

In another embodiment, R⁴ is lower alkyl.

In another embodiment, R¹⁰ is a 4-8-membered heterocyclyl ring having from 1 to 3 ring N atoms, wherein said heterocyclyl ring is substituted off of a ring N atom.

In another embodiment, R¹⁰ is a 4-8-membered heterocyclyl ring having from 1 to 3 ring N atoms, wherein said heterocyclyl ring is substituted off of a ring carbon atom.

In another embodiment, R¹⁰ is a bicyclic heterocyclyl ring having from 1 to 3 ring N atoms, wherein said bicyclic heterocyclyl ring is substituted off of a ring N atom.

In another embodiment, R¹⁰ is a bicyclic heterocyclyl ring having from 1 to 3 ring N atoms, wherein said bicyclic heterocyclyl ring is substituted off of a ring carbon atom.

In another embodiment, R¹⁰ is a 4-8-membered heterocyclyl ring having from 1 to 3 ring N atoms, wherein said heterocyclyl ring is substituted with G, wherein G is as previously described.

In another embodiment, R¹⁰ is a 4-8-membered heterocyclyl ring having from 1 to 3 ring N atoms, wherein said heterocyclyl ring is fused with a heteroaryl ring, wherein said R¹⁰ is optionally substituted with G, wherein G is as previously described.

In another embodiment, R¹⁰ is the moiety:

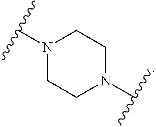

In another embodiment, R¹⁰ is the moiety:

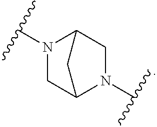

In another embodiment, R¹⁰ is the moiety:

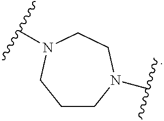

In another embodiment, R¹⁰ is the moiety:

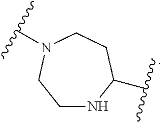

In another embodiment, R¹⁰ is a piperidinyl ring, wherein said piperidinyl ring is substituted with G, wherein G is as previously described.

In another embodiment, R¹⁰ is a piperazinyl ring, wherein said piperazinyl ring is with G, wherein G is as previously described.

In another embodiment, R¹⁰ is a diazepinyl ring, wherein said diazepinyl ring is substituted with G, wherein G is as previously described.

In another embodiment, R¹⁰ is a diazepinyl ring, wherein said diazepinyl ring is substituted with two G moieties, wherein G is as previously described.

In another embodiment, G is

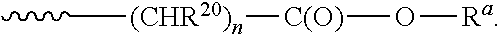

In another embodiment, G is

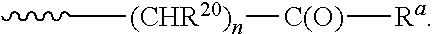

In another embodiment, G is

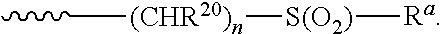

In another embodiment, G is

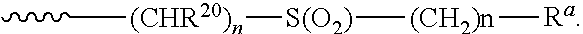

In another embodiment, G is

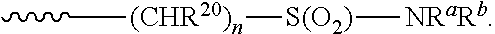

In another embodiment, G is

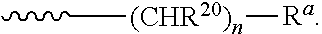

In another embodiment, G is

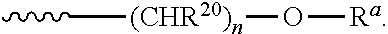

In another embodiment, G is

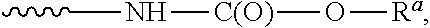

with the proviso described earlier.

In another embodiment, G is

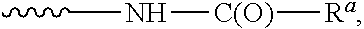

with the proviso described earlier.

In another embodiment, G is

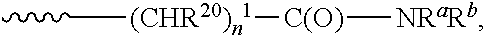

with the proviso described earlier.

In another embodiment, G is

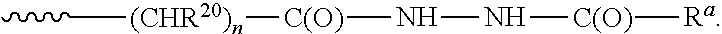

In another embodiment, G is

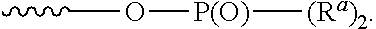

In another embodiment, G is

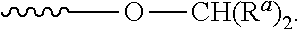

In another embodiment. G is an oxo group.

In another embodiment, G is

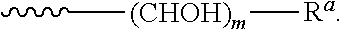

In another embodiment, G is

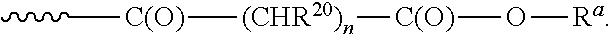

In another embodiment, G is

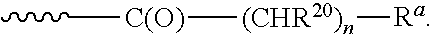

In another embodiment, G is a spirocyclyl group.

In another embodiment, G is the moiety:

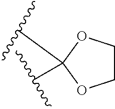

coming off of a carbon atom of R¹⁰.

In another embodiment, R^{a} is unsubstituted alkyl.

In another embodiment, R^{a} is alkyl substituted as previously described under formula I.

In another embodiment, R^{a} is unsubstituted aryl.

In another embodiment. R^{a} is aryl substituted as previously described under formula I.

In another embodiment, R^{a} is unsubstituted heteroaryl.

In another embodiment, R^{a} is heteroaryl substituted as previously described under formula I.

In another embodiment, R^{a} is unsubstituted cycloalkyl.

In another embodiment, R^{a} is cycloalkyl substituted as previously described under formula I.

In another embodiment, R^{a} is unsubstituted heterocyclyl.

In another embodiment, R^{a} is heterocyclyl substituted as previously described under formula I.

In another embodiment, R^{a} is hydroxy.

In another embodiment, R^{a} is cyano.

In another embodiment, R^{a} is halo.

In another embodiment, R^{a} is alkeny.

In another embodiment, R^{a} is alkynyl.

In another embodiment, R^{a} is alkoxyalkyl.

In another embodiment, R^{a} is aralkyl.

In another embodiment, R^{a} is haloalkyl.

In another embodiment, R^{a} is CF₃.

In another embodiment, R^{a} is phenyl substituted with one or more halo groups.

In another embodiment. R^{a} is heteroaryl.

In another embodiment, R^{a} is pyridyl.

In another embodiment, R^{a} is oxazolyl.

In another embodiment, R^{a} is oxadiazolyl.

In another embodiment, the moiety:

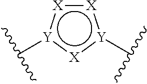

is selected from the group consisting of the following moieties:

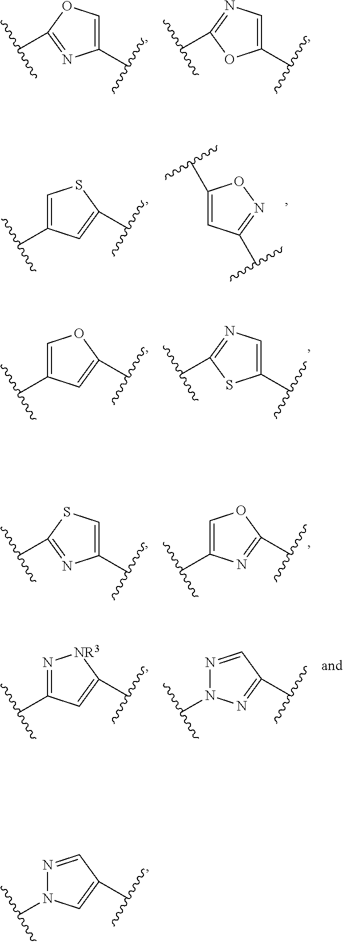

as well as their possible positional isomers, these moieties being unsubstituted or optionally substituted with R³.

In another embodiment, in Formula I, the moiety:

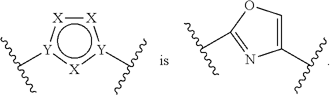

In another embodiment, in Formula I, the moiety:

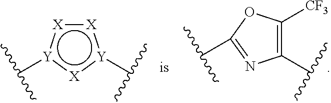

In another embodiment, in Formula I, the moiety:

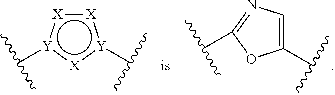

In another embodiment, in Formula I, the moiety:

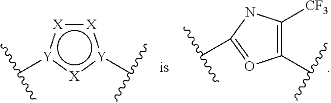

In another embodiment, in Formula the moiety:

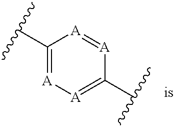

selected from the group consisting of the following moieties:

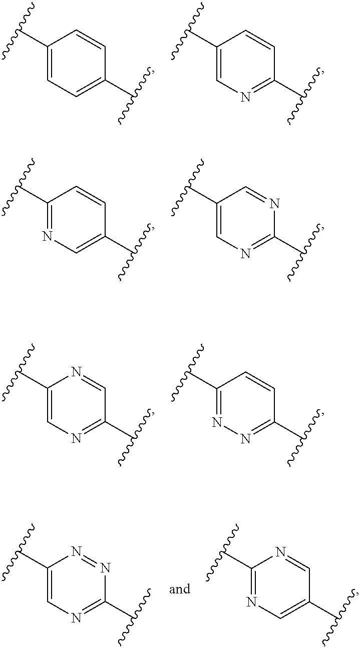

as well as any of their positional isomers.

In another embodiment, in Formula I, the moiety:

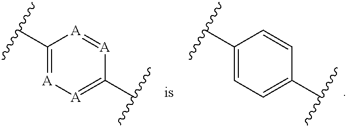

In another embodiment, in Formula I, the moiety:

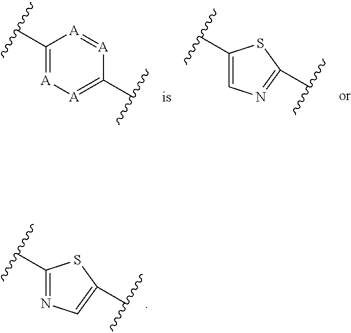

In another embodiment, in Formula I, the moiety:

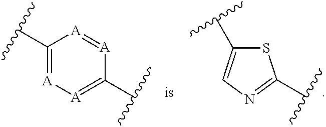

In another embodiment, in Formula I, the moiety:

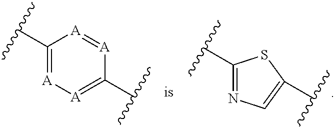

In another embodiment, in Formula I, the moiety:

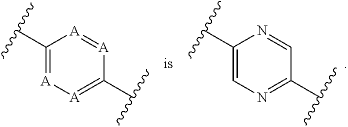

In another embodiment, in Formula I, the moiety:

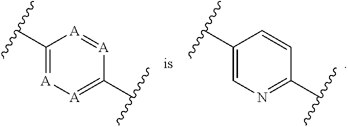

In another embodiment, in Formula I the moiety:

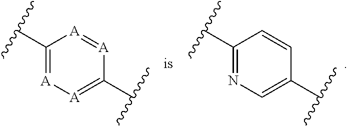

In another embodiment, in Formula I, the moiety:

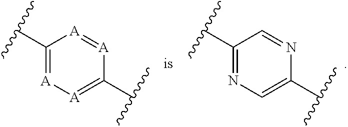

In another embodiment, Z is a bond. Z is a bond means that R¹⁰ is directly linked to the

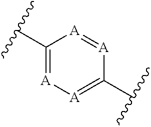

ring.

In another embodiment, Z is O.

In another embodiment, Z is NR⁴.

In another embodiment, p is 0.

In another embodiment, p is 1.

In another embodiment of Formula I, wherein X, Y, R¹, A, R¹⁰, R^{a} and the other moieties are independently selected, one X is N, a second X is C, and the third X is O, both Y are C, one A is N and the other A moieties are C, R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is alkyl, R¹⁰ is piperidinyl ring and R^{a} is as previously described.

In another embodiment of Formula I, wherein X, Y, R¹, A, R¹⁰, R^{a} and the other moieties are independently selected, one X is N a second X is C, and the third X is O, both Y are C, one A is N and the other A's are C, R¹ is aryl substituted as described previously under Formula I, R¹⁰ is piperidinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is aryl substituted as described previously under Formula I, R³ is alkyl, R¹⁰ is piperidinyl ring and R^{a} is as previously described.

In another embodiment of Formula I, wherein X, Y, R¹, A, R¹⁰, R^{a} and the other moieties are independently selected, one X is N, a second X is C, and the third X is O, both Y are C, one A is N and the other A's are C, R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is alkyl, R¹⁰ is piperazinyl ring and R^{a} is as previously described.

In another embodiment of Formula I, wherein X, Y, R¹, A, R¹⁰, R^{a} and the other moieties are independently selected, one X is N, a second X is C, and the third X is O, both Y are C, one A is N and the other A's are C, R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is aryl substituted as described previously under Formula I, R³ is alkyl, R¹⁰ is piperazinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is haloalkyl, R¹⁰ is piperidinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is haloalkyl, R¹⁰ is piperazinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is —CN, R¹⁰ is piperidinyl ring and R^{a} is as previously described.

In another embodiment of Formula I, wherein X, Y, R¹, A, R¹⁰, R^{a} and the other moieties are independently selected, one X is N, a second X is C(R³), and the third X is O, both Y are C, one A is N and the other A's are C, R¹ is unsubstituted aryl, R³ is —CN, R¹⁰ is piperazinyl ring and R^{a} is as previously described.

In another embodiment of Formula I, wherein X, Y, R¹, A, R¹⁰, R^{a} and the other moieties are independently selected, the moiety:

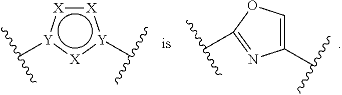

one A is N and the other A's are C, R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

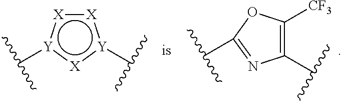

one A is N and the other A's are C, R¹ is unsubstituted aryl, R¹⁰ is piperazinyl ring and R^{a} is as previously described. In another embodiment of Formula I, wherein X, Y, R¹, A, R¹⁰, R^{a} and the other moieties are independently selected, the moiety:

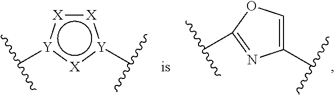

one A is N and the other A's are C, R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

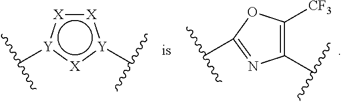

one A is N and the other A's are C, R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

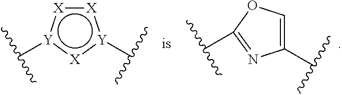

one A is N and the other A's are C, R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

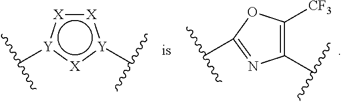

one A is N and the other A's are C, R¹ is aryl substituted as previously described under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

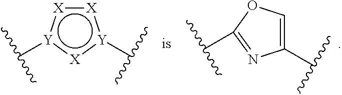

the moiety:

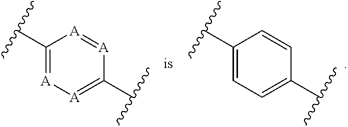

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

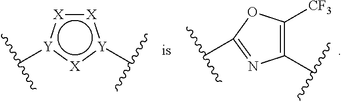

the moiety:

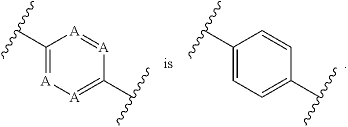

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

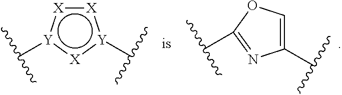

the moiety:

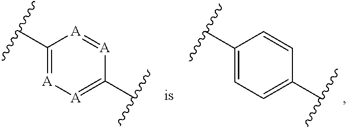

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

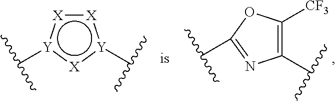

the moiety:

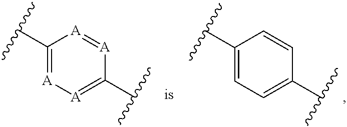

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

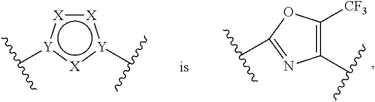

the moiety:

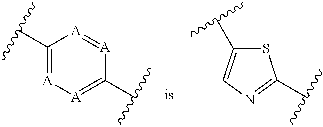

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

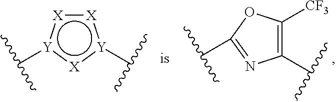

the moiety:

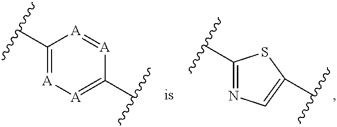

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

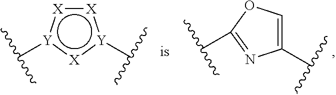

one A is N and the other A's are C, R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

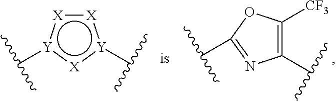

one A is N and the other A's are C, R¹ is aryl substituted as previously described under Formula I, R¹⁰ is piperidinyl ring and R^{a} is as previously described.

In another embodiment of Formula I, wherein X, Y, R¹, A, R¹⁰, R^{a} and the other moieties are independently selected, the moiety:

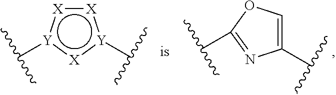

the moiety:

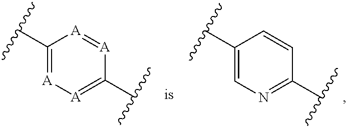

R¹ is aryl substituted as described previously under Formula I, R¹⁰ is piperidinyl ring and R^{a} is as previously described.

In another embodiment of Formula I, wherein X, Y, R¹, A, R¹⁰, R^{a} and the other moieties are independently selected, the moiety:

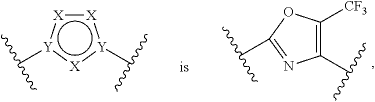

the moiety:

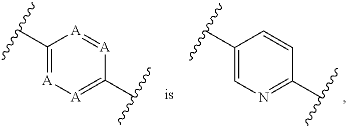

R¹ is aryl substituted as described previously under Formula I, R¹⁰ is piperidinyl ring and R^{a} is as previously described.

In another embodiment of Formula I, wherein X, Y, R¹, A, R¹⁰, R^{a} and the other moieties are independently selected, the moiety:

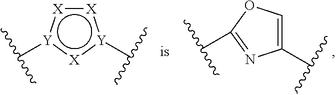

the moiety:

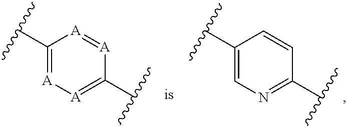

R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

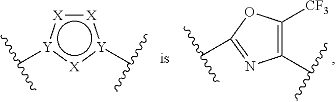

the moiety:

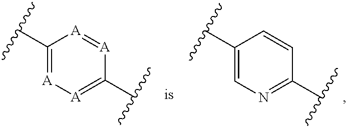

R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

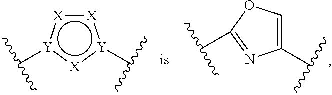

the moiety:

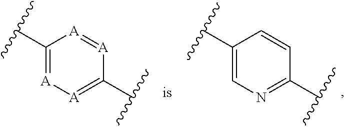

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

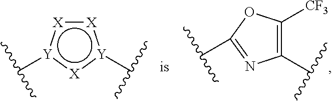

the moiety:

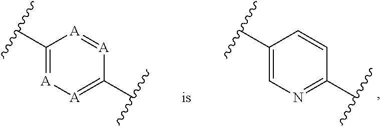

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

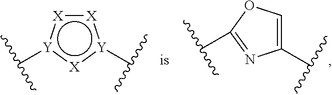

the moiety:

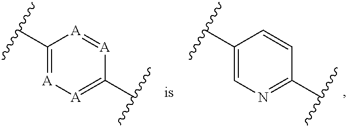

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

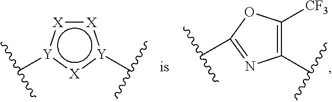

the moiety:

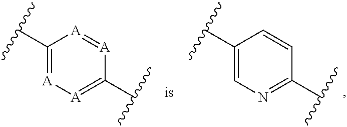

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

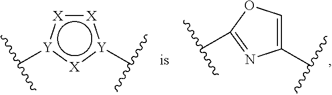

the moiety:

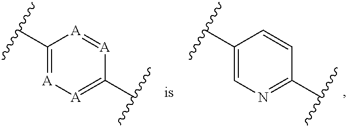

R¹ is aryl substituted as described previously under Formula I, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

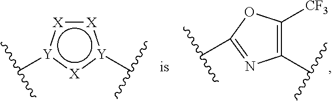

the moiety:

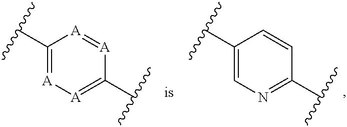

R¹ is aryl substituted as described previously under Formula I, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

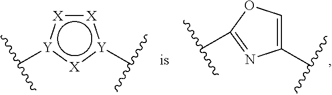

the moiety:

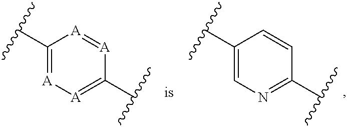

R¹ is aryl substituted as described previously under Formula I, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

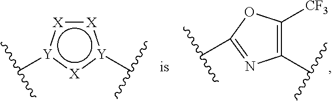

the moiety:

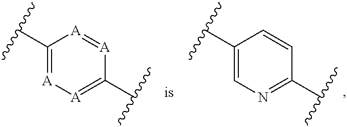

R¹ is aryl substituted as described previously under Formula I, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

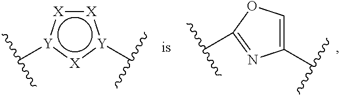

the moiety:

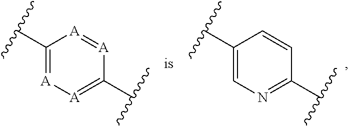

R¹ is unsubstituted aryl, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

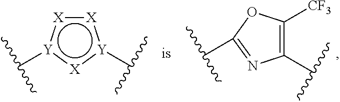

the moiety:

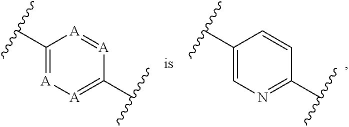

R¹ is unsubstituted aryl, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

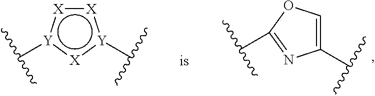

the moiety:

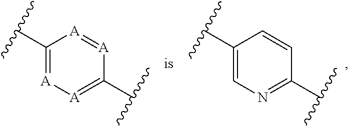

R¹ is unsubstituted aryl, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

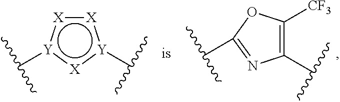

the moiety:

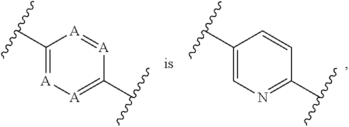

R¹ is unsubstituted aryl, R¹⁰ is piperazinyl with —C(O)—O—R^{a}, and R^{a} is as previously described.

In another embodiment of Formula I, wherein X, Y, R¹, A, R¹⁰, R^{a} and the other moieties are independently selected, the moiety:

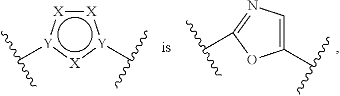

one A is N and the other A's are C, R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

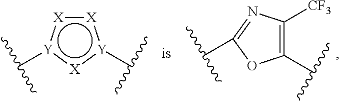

one A is N and the other A's are C, R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

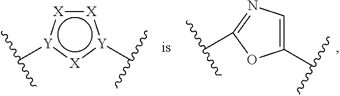

one A is N and the other A's are C, R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

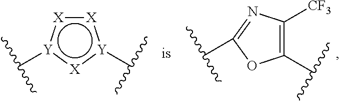

one A is N and the other A's are C, R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

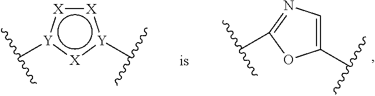

one A is N and the other A's are C, R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

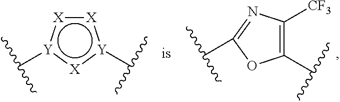

one A is N and the other A's are C, R¹ is aryl substituted as previously described under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

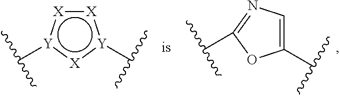

the moiety:

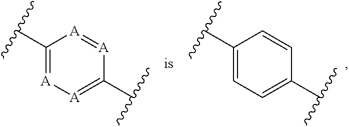

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

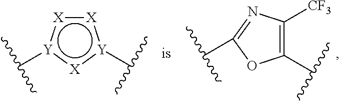

the moiety:

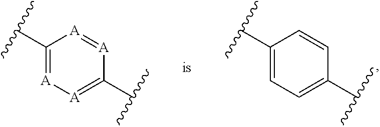

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

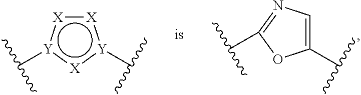

the moiety:

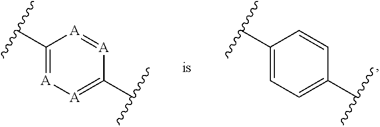

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

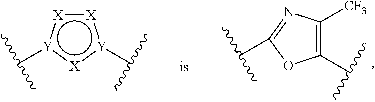

the moiety:

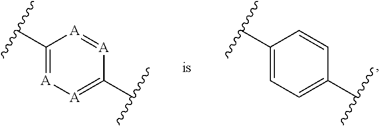

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

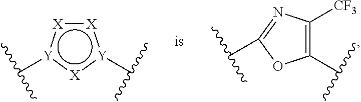

the moiety:

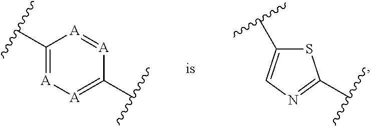

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

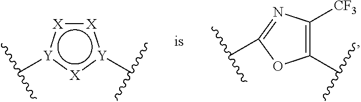

the moiety:

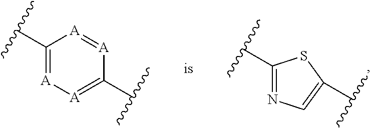

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

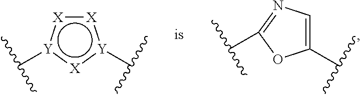

one A is N and the other A's are C, R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

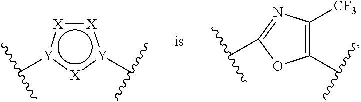

one A is N and the other A's are C, R¹ is aryl substituted as previously described under Formula I, R¹⁰ is piperidinyl ring and R^{a} is as previously described.

In another embodiment of Formula i, wherein X, Y, R¹, A, R¹⁰, R^{a} and the other moieties are independently selected, the moiety:

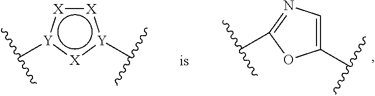

the moiety:

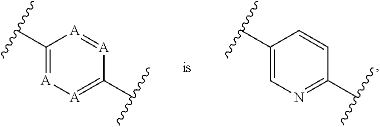

R¹ is aryl substituted as described previously under Formula I, R¹⁰ is piperidinyl ring and R^{a} is as previously described.

In another embodiment of Formula I, wherein X, Y, R¹, A, R¹⁰, R^{a} and the other moieties are independently selected, the moiety:

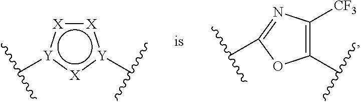

the moiety:

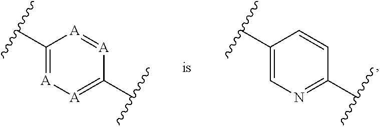

R¹ is aryl substituted as described previously under Formula I, R¹⁰ is piperidinyl ring and R^{a} is as previously described.

In another embodiment of Formula I, wherein X, Y, R¹, A, R¹⁰, R^{a} and the other moieties are independently selected, the moiety:

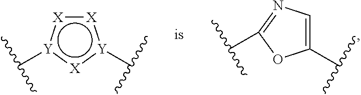

the moiety:

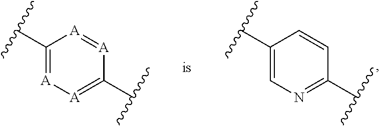

R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

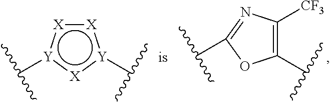

the moiety:

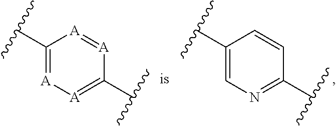

R¹ is aryl substituted as described previously under Formula I, R¹⁰ is piperazinyl ring and R^{a} is as previously described.

In another embodiment of Formula I, wherein X, Y, R¹, A, R¹⁰, R^{a} and the other moieties are independently selected, the moiety:

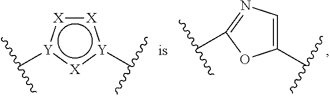

the moiety:

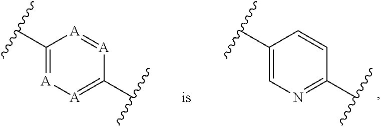

R¹ is unsubstituted aryl, R¹⁰ is piperidinyl ring and R^{a} is as previously described.

In another embodiment of Formula wherein X, Y, R¹, A, R¹⁰, R^{a} and the other moieties are independently selected, the moiety:

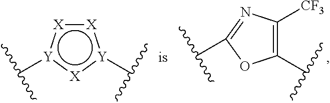

the moiety:

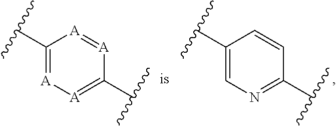

R¹ is unsubstituted aryl, R¹⁰ is piperidinyl ring and R^{a} is as previously described.

In another embodiment of Formula I, wherein X, Y, R¹, A, R¹⁰, R^{a} and the other moieties are independently selected, the moiety:

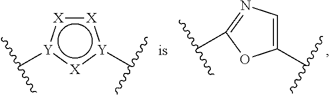

the moiety:

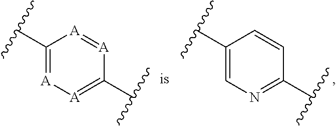

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

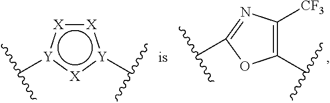

the moiety:

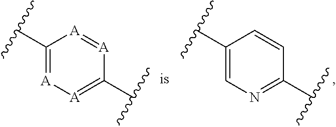

R¹ is unsubstituted aryl, R¹⁰ is piperazinyl ring and R^{a} is as previously described.

In another embodiment of Formula I, wherein X, Y, R¹, A, R¹⁰, R^{a} and the other moieties are independently selected, the moiety:

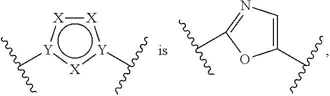

the moiety:

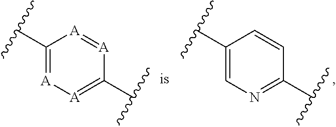

R¹ is aryl substituted as described previously under Formula I, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

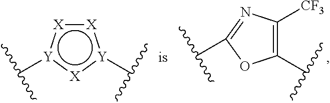

the moiety:

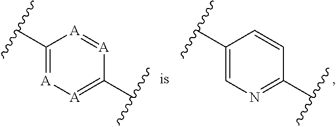

R¹ is aryl substituted as described previously under Formula I, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

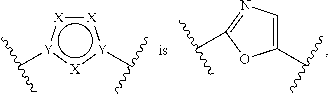

the moiety:

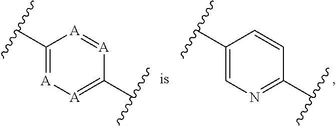

R¹ is aryl substituted as described previously under Formula I, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

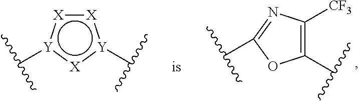

the moiety:

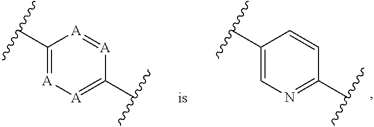

R¹ is aryl substituted as described previously under Formula I, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

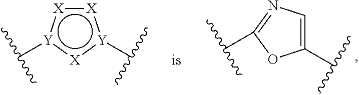

the moiety:

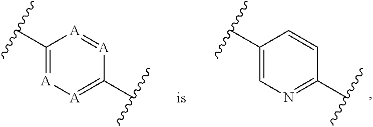

R¹ is unsubstituted aryl, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

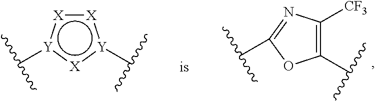

the moiety:

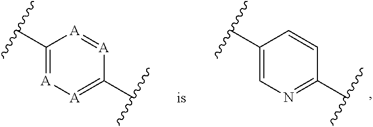

R¹ is unsubstituted aryl, R¹⁰ is piperidinyl ring with —C(O)—O—R^{a}, and R^{a} is as previously described.

In another embodiment of Formula I, wherein X, Y, R¹, A, R¹⁰, R^{a} and the other moieties are independently selected, the moiety:

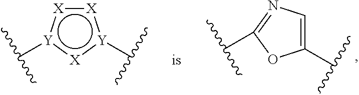

the moiety:

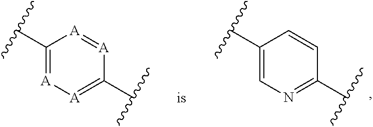

R¹ is unsubstituted aryl, R¹⁰ is piperazinyl ring with —C(O)—O—R^{a}, and R^{a} is as previously described.

In another embodiment of Formula I, wherein X, Y, R¹, A, R¹⁰, R^{a} and the other moieties are independently selected, the moiety:

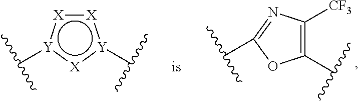

the moiety:

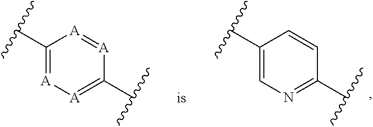

R¹ is unsubstituted aryl, R¹⁰ is piperazinyl with —C(O)—O—R^{a}, and R^{a} is as previously described.

Non-limiting examples of the compounds of Formula I are shown below:

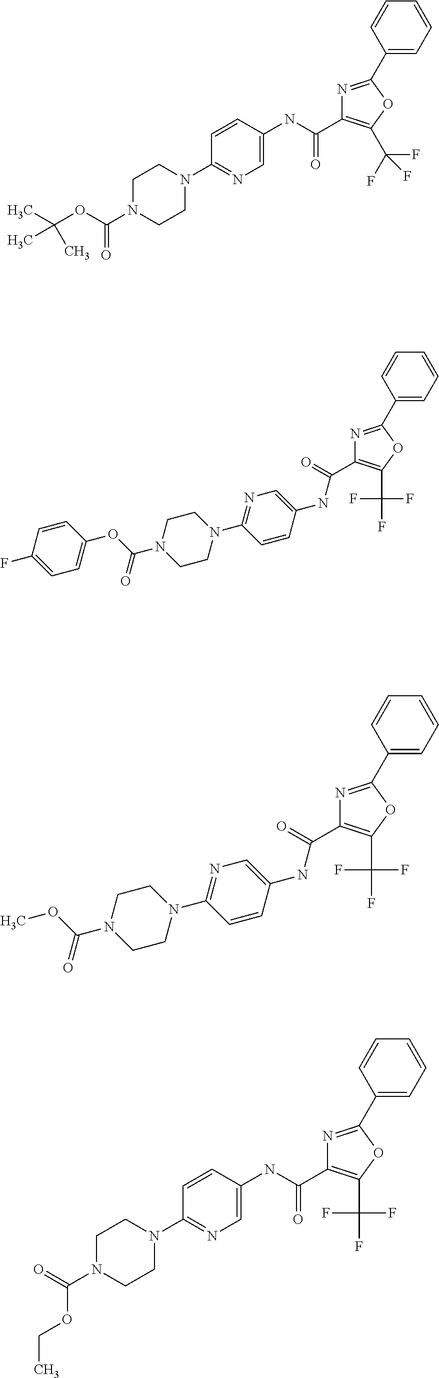

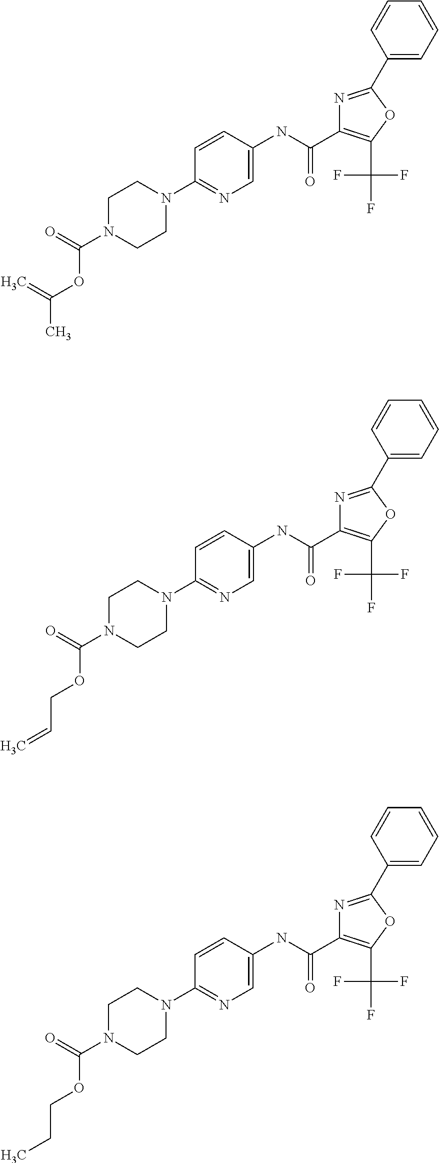

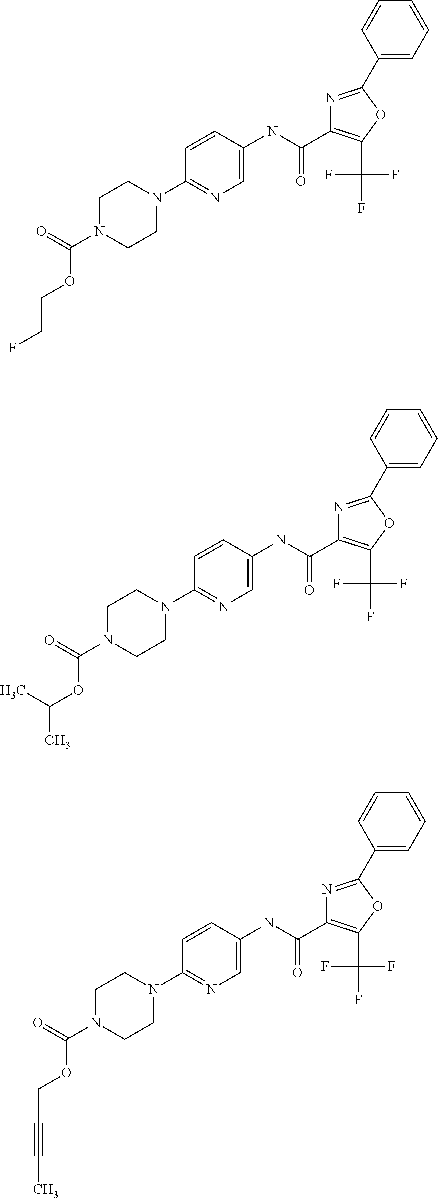

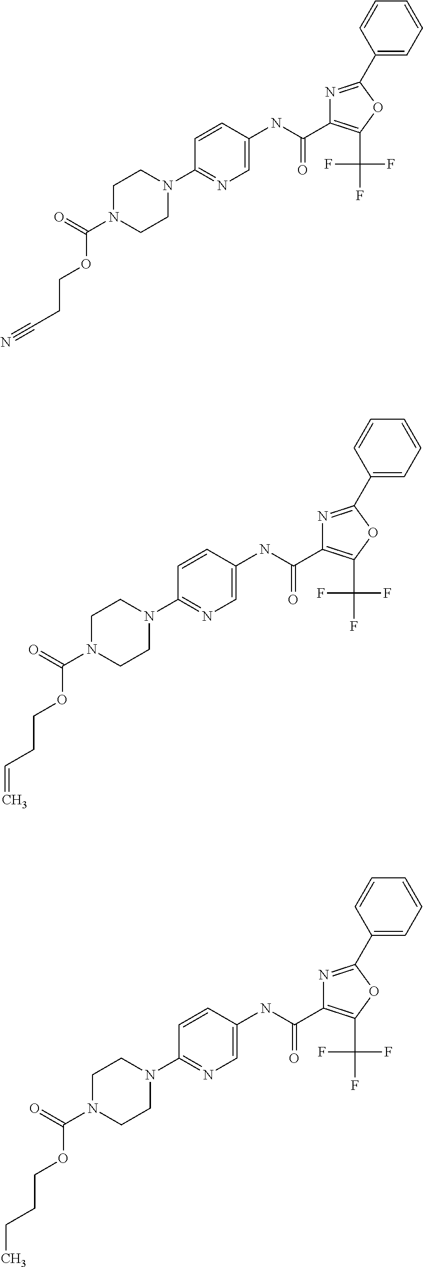

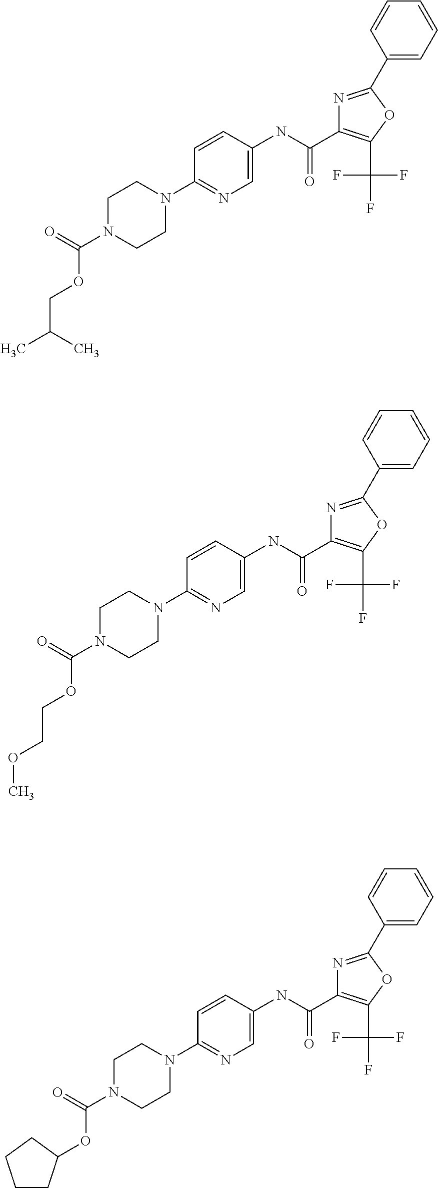

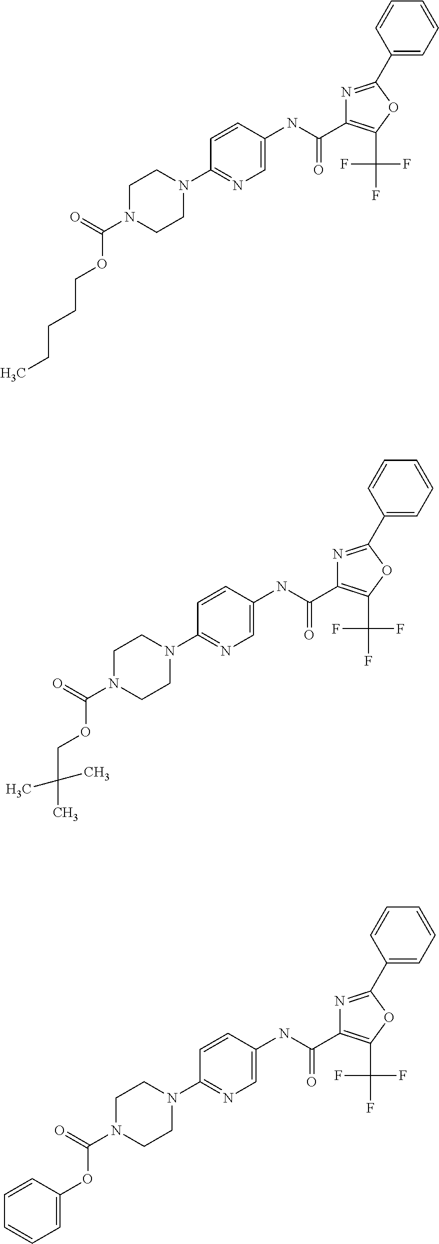

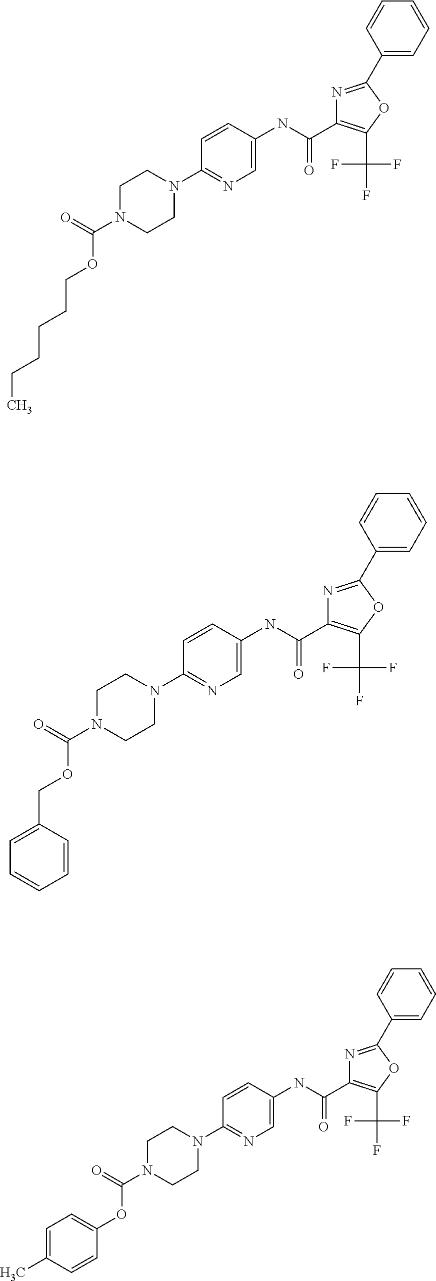

The above-noted compounds exhibited IC₅₀ values less than 3 μM in the assay described later.

As used above, and throughout this disclosure, the following terms, unless otherwise indicated, shall be understood to have the following meanings:

“Patient” includes both humans and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more tower alkyl groups such as methyl, ethyl or propyl, are attached to a linear alkyl chain. Lower alkyl means a group having about 1 to about 6 carbon atoms in the chain which may be straight or branched. Alkyl may be unsubstituted or optionally substituted by one or more substituents which may be the same or different, each substituent being independently selected from the group consisting of halo, alkyl, aryl, cycloalkyl, cyano, pyridine, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. Lower alkenyl means about 2 to about 6 carbon atoms in the chain which may be straight or branched. Alkenyl may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Lower alkynyl means about 2 to about 6 carbon atoms in the chain which may be straight or branched. Non-limiting examples of suitable alkynyl groups include ethynyl, propynyl, 2-butyryl and 3-methylbutynyl. Alkynyl may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridine (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” or “halo” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, methylenedioxy, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl, “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

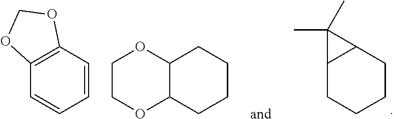

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic (e.g. bicyclic) ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, diazepinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

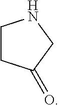

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

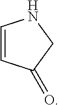

“Heterocyclenylalkyl” means a heterocyclenyl moiety as defined above linked via an alkyl moiety (defined above) to a parent core.

It should be noted that in heteroatom containing ring systems of this invention, there are no hydroxyl groups on carbon atoms adjacent to a N, O or S, as well as there are no N or S groups on carbon adjacent to another heteroatom. Thus, for example, in the ring:

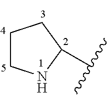

there is no —OH attached directly to carbons marked 2 and 5.

It should also be noted that tautomeric forms such as, for example, the moieties:

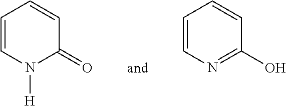

are considered equivalent in certain embodiments of this invention,

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)-group in which the aryl group is as previously described. The bond to the parent moiety is through the carbonyl. Non-limiting examples of suitable groups include benzoyl and 1-naphthoyl.

“Alkoxy” means an alkyl-O-group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkoxyalkyl-” means an alkyl-O-alkyl-group in which the alkyl group is as previously described. Non-limiting examples of suitable alkoxyalkyl groups include methoxymethyl, ethoxymethyl, n-propoxyethyl, isopropoxyethyl and n-butoxymethyl. The bond to the parent moiety is through the alkyl-.

“Aryloxy” means an aryl-O-group in which the aryl group is as previously described. Non-limiting examples of suitable aryloxy groups include phenoxy and naphthoxy. The bond to the parent moiety is through the ether oxygen.

“Aryloxyalkyl-” means an aryl-O-alkyl-group in which the aryl and aryl groups are as previously described. Non-limiting examples of suitable aryloxyalkyl groups include phenoxymethyl and naphthoxyethyl. The bond to the parent moiety is through the alkyl.

“Aralkyloxy” means an aralkyl-O-group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S-group in which the alkyl group is as previously described. Non-limiting examples of suitable alkylthio groups include methylthio and ethylthio. The bond to the parent moiety is through the sulfur.

“Alkylthioalkyl-” means an alkyl-S-alkyl-group in which the alkyl group is as previously described. Non-limiting examples of suitable alkylthioalkyl groups include methylthioethyl and ethylthiomethyl. The bond to the parent moiety is through the alkyl.

“Arylthio” means an aryl-S-group in which the aryl group is as previously described. Non-limiting examples of suitable arylthio groups include phenylthio and naphthylthio. The bond to the parent moiety is through the sulfur.

“Arylthioalkyl-” means an aryl-S-alkyl-group in which the aryl group is as previously described. Non-limiting examples of suitable arylthioalkyl groups include phenylthioethyl and phenylthiomethyl. The bond to the parent moiety is through the alkyl.

“Aralkylthio” means an aralkyl-S-group in which the aralkyl group is as previously described. Non-limiting example of a suitable aralkylthio group is benzylthio. The bond to the parent moiety is through the sulfur.

“Alkoxycarbonyl” means an alkyl-O—CO-group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)-group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)-group. Non-limiting example of a suitable aralkoxycarbonyl group is benzyloxycarbonyl. The bond to the parent moiety is through the carbonyl.

“Alkylsulfonyl” means an alkyl-S(O₂)-group. Preferred groups are those in which the alkyl group is lower alkyl. The bond to the parent moiety is through the sulfonyl.

“Arylsulfonyl” means an aryl-S(O₂)-group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula I or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates, Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., (2004) 93(3), pp. 601-611 describe the preparation of the solvates of the antifungal fluconazole in ethyl acetate as well as from water. Similar preparations of solvates, hemisolvate, hydrates and the like are described by E. C. van Tender et al, AAPS PharmSciTech., (2004) 5(1), article 12; and A. L. Bingham et al, Chem. Commun., (2001) pp. 603-604. A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “effective” or “therapeutically effective” is used herein, unless otherwise indicated, to describe an amount of a compound or composition which, in context, is used to produce or effect an intended result or therapeutic effect as understood in the common knowledge of those skilled in the art.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et at, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) pp. 1-19; P. Gould, International J. of Pharmaceutics (1986) (2001) 33 pp. 201-217; Anderson et al, The Practice of Medicinal Chemistry (1996). Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula I may contain asymmetric or chiral centers, and, therefore, exist in different stereoisomeric forms. It is intended that all stereoisomeric forms of the compounds of Formula I as well as mixtures thereof, including racemic mixtures, form part of the present invention. In addition, the present invention embraces all geometric and positional isomers. For example, if a compound of Formula I incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula I may be atropisomers (e.g., substituted biaryls) and are considered as part of this invention. Enantiomers can also be separated by use of chiral HPLC column.

It is also possible that the compounds of Formula I may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula I incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention further includes the compounds of the invention in their isolated forms.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention have pharmacological properties. The compounds of Formula I are inhibitors of DGAT, particularly DGAT1, and can be useful for the therapeutic and/or prophylactic treatment of diseases that are modulated by DGAT, particularly by DGAT1, such as, for example, metabolic syndrome, diabetes (e.g., Type 2 diabetes mellitus), obesity and the like.

The invention also includes methods of treating diseases that are modulated by DGAT, particularly by DGAT1.

The invention also includes methods of treating metabolic syndrome, diabetes (e.g., Type 2 diabetes mellitus), and obesity in a patient by administering at least one compound of Formula I to said patient.

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of the lipid, lipoprotein and apolipoprotein metabolism and other metabolic and hemodynamic disease. As such, the diabetic patient is at especially increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In Type 1 diabetes, or insulin-dependent diabetes mellitus (IDDM), patients produce little or no insulin, the hormone which regulates glucose utilization. In Type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for Type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a class of agents that can increase insulin sensitivity and bring about some degree of correction of hyperglycemia. However, the biguanides can induce lactic acidosis and nausea/diarrhea.

The glitazones (i.e. 5-benzylthiazolidine-2,4-diones) are a separate class of compounds with potential for the treatment of Type 2 diabetes. These agents increase insulin sensitivity in muscle, liver and adipose tissue in several animal models of Type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensitization that is observed with the glitazones. Newer PPAR agonists that are being tested for treatment of Type 2 diabetes are agonists of the alpha, gamma or delta subtype, or a combination of these, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly Type 2 diabetes.

The invention includes compositions, e.g., pharmaceutical compositions, comprising at least one compound of Formula I. For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Other carriers include Poloxamer, Povidone K17, Povidone K12, Tween 80, ethanol, Cremophor/ethanol, polyethylene glycol (PEG) 400, propylene glycol, Trappsol, alpha-cyclodextrin or analogs thereof, beta-cyclodextrin or analogs thereof, or gamma-cyclodextrin or analogs thereof. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The therapeutic agents of the present invention are preferably formulated in pharmaceutical compositions and then, in accordance with the methods of the invention, administered to a subject, such as a human subject, in a variety of forms adapted to the chosen route of administration. For example, the therapeutic agents may be formulated for intravenous administration. The formulations may, however, include those suitable for oral, rectal, vaginal, topical, nasal, ophthalmic, or other parenteral administration (including subcutaneous, intramuscular, intrathecal, intraperitoneal and intratumoral, in addition to intravenous) administration.

Formulations suitable for parenteral administration conveniently include a sterile aqueous preparation of the active agent, or dispersions of sterile powders of the active agent, which are preferably isotonic with the blood of the recipient. Parenteral administration of the therapeutic agents (e.g., through an I.V. drip) is an additional form of administration. Isotonic agents that can be included in the liquid preparation include sugars, buffers, and sodium chloride. Solutions of the active agents can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the first and/or second therapeutic agents, or as a solution or suspension in an aqueous liquor or non-aqueous liquid such as a syrup, an elixir, an emulsion, or a draught. Such compositions and preparations may contain at least about 0.1 wt-% of the active agent. The amounts of the therapeutic agents should be such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

Preferably the compound is administered orally, intraperitoneally, or intravenously or intrathecally or some suitable combination(s) thereof.

Methods of administering small molecule therapeutic agents are well-known in the art.

The therapeutic agents described in the present disclosure can be administered to a subject alone or together (coadministered, optionally but not necessarily, in a single formulation) with other active agents as described herein, and are preferably administered with a pharmaceutically acceptable buffer. The therapeutic agents can be combined with a variety of physiological acceptable carriers, additives for delivery to a subject, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the therapeutic agent (i.e., the active agent) into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering the therapeutic agents to a subject in an amount effective to produce the desired effect. The therapeutic agents can be administered as a single dose or in multiple doses. Useful dosages of the active agents can be determined by comparing their in vitro activity and the in vivo activity in animal models.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Another aspect of the invention includes pharmaceutical compositions comprising at least one compound of Formula I and at least one other therapeutic agent in combination. Non-limiting examples of such combination agents are described below. The agents in the combination can be administered together as a joint administration (e.g., joint single pill), separately, one after the other in any order and the like as is well known in the art.

In the combination therapies of the present invention, an effective amount can refer to each individual agent or to the combination as a whole, wherein the amounts of all agents administered are together effective, but wherein the component agent of the combination may not be present individually in an effective amount.

### Combination Therapy

Accordingly, in one embodiment, the present invention provides methods for treating a Condition in a patient, the method comprising administering to the patient one or more Compounds of Formula I, or a pharmaceutically acceptable salt or solvate thereof and at least one additional therapeutic agent that is not a Compound of Formula I, wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during a time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer (such as a PPAR agonist, a DPP-IV inhibitor, a PTP-1B inhibitor and a glucokinase activator); a glucosidase inhibitor; an insulin secretagogue; a hepatic glucose output lowering agent; an anti-obesity agent; a meglitinide; an agent that slows or blocks the breakdown of starches and sugars in vivo; an histamine H₃ receptor antagonist; a sodium glucose uptake transporter 2 (SGLT-2) inhibitor; a peptide that increases insulin production; and insulin or any insulin-containing composition.

In one embodiment, the antidiabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

Non-limiting examples of insulin sensitizers include PPAR activators, such as rosiglitazone, pioglitazone and englitazone; biguanidines such as metformin and phenformin; DPP-IV inhibitors; PTP-1B inhibitors; and α-glucokinase activators, such as miglitol, acarbose, and voglibose.

Non-limiting examples of DPP-IV inhibitors useful in the present methods include sitagliptin (Januvia™, Merck), saxagliptin, denagliptin, vildagliptin (Galvus™, Novartis), alogliptin, alogliptin benzoate, ABT-279 and ABT-341 (Abbott), ALS-2-0426 (Alantos), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

Non-limiting examples of SGLT-2 inhibitors useful in the present methods include dapagliflozin and sergliflozin, AVE2268 (Sanofi-Aventis) and 1-1095 (Tanabe Seiyaku).

Non-limiting examples of hepatic glucose output lowering agents include Glucophage and Glucophage XR.

Non-limiting examples of histamine H₃ receptor antagonist agents include the following compound;

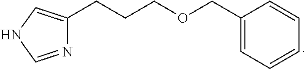

Non-limiting examples of insulin secretagogues include sulfonylurea and non-sulfonylurea drugs such as GLP-1, a GLP-1 mimetic, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exenatide, Liraglutide, CJC-1131 (ConjuChem, Exenatide-LAR (Amylin), BIM-51077 (Ipsen/LaRoche), ZP-10 (Zealand Pharmaceuticals), and compounds disclosed in International Publication No. WO 00/07617.

The term “insulin” as used herein, includes all pyridinones of insulin, including long acting and short acting forms of insulin.

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

In one embodiment, the antidiabetic agent is an anti-obesity agent.

Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

Non-limiting examples of meglitinides useful in the present methods for treating diabetes include repaglinide and nateglinide.

Non-limiting examples of insulin sensitizing agents include biguanides, such as metformin, metformin hydrochloride (such as GLUCOPHAGE® from Bristol-Myers Squibb), metformin hydrochloride with glyburide (such as GLUCOVANCE™ from Bristol-Myers Squibb) and buformin; glitazones; and thiazolidinediones, such as rosiglitazone, rosiglitazone maleate (AVANDIA™ from GlaxoSmithKline), pioglitazone, pioglitazone hydrochloride (ACTOS™ from Takeda) ciglitazone and MCC-555 (Mitsubishi Chemical Co.)

In one embodiment, the insulin sensitizer is a thiazolidinedione.

In another embodiment, the insulin sensitizer is a biguanide.

In another embodiment, the insulin sensitizer is a. DPP-IV inhibitor.

In a further embodiment, the antidiabetic agent is a SGLT-2 inhibitor.

Non-limiting examples of antidiabetic agents that slow or block the breakdown of starches and sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

Generally, a total daily dosage of the one or more Compounds of Formula (I) and the additional therapeutic agent(s) can, when administered as combination therapy, range from about 0.1 to about 2000 mg per day, although variations will necessarily occur depending on the target of the therapy, the patient and the route of administration. In one embodiment, the dosage is from about 0.2 to about 1000 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In a further embodiment, the dosage is from about 1 to about 20 mg/day, administered in a single dose or in 2-4 divided doses.

The compounds of the invention can be made according to the processes described below. The compounds of this invention are also exemplified in the examples below, which examples should not be construed as limiting the scope of the disclosure. Alternative mechanistic pathways and analogous structures within the scope of the invention may be apparent to those skilled in the art.

### General Procedure for the Hydrolysis of Ethyl Esters.

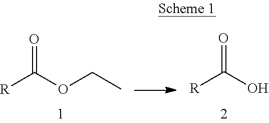

To a solution of ethyl ester 1 (0.037 mmol) in tetrahydrofuran (1 mL) was added water (0.5 mL) and lithium hydroxide monohydrate (0.14 mmol). The reaction mixture was stirred at room temperature for 4 h. Ethyl acetate and water was added. The organic layer was washed with 10% citric acid solution. The organic layer was dried over sodium sulfate. The organic solvent was evaporated under reduced pressure. The crude product was purified by reversed phase HPLC to yield the desired carboxylic acid 2.

### General Procedure for the Hydrolysis of Boc Protected Amines.

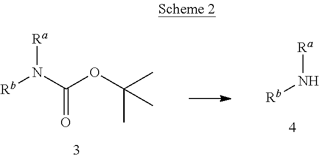

To a solution of the Boc protected compound 3 (0.16 mmol) in dichloromethane (1.6 mL) was added trifluoroacetic acid (0.25 mL). The reaction mixture was stirred at room temperature for 16 h. The organic solvent was evaporated under reduced pressure. The crude product was purified by reversed phase HPLC to yield the desired amine 4.

### General Procedure for the Formation of Carbamate Compounds.

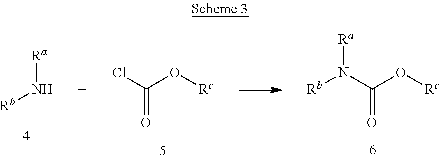

To a solution of amine 4 (0.17 mmol in dichloromethane (2 mL) was added chloroformate 5 (0.21 mmol) and triethylamine (0.6 mmol). The reaction mixture was stirred at room temperature for 2 h. Dichloromethane and water was added. The organic layer was washed with water and brine. The organic layer was dried over anhydrous sodium sulfate. The organic solvent was evaporated under reduced pressure. The crude product was purified by flash column chromatography to yield the desired carbamate 6.

### General Procedure for the Formation of Sulfonamide Compounds.

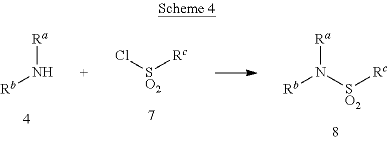

To a solution of amine 4 (0.079 mmol) in dichloromethane (2 mL) was added sulfonyl chloride 7 (0.084 mmol) and triethylamine (0.28 mmol). The reaction mixture was stirred at room temperature for 2 h. Dichloromethane and water was added. The organic layer was washed with water and brine. The organic layer was dried over anhydrous sodium sulfate. The organic solvent was evaporated under reduced pressure. The crude product was purified by flash column chromatography to yield the desired sulfonamide 8.

### General Procedure for the Formation of Urea Compounds.

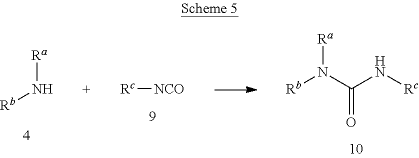

Parallel syntheses were conducted in polypropylene tubes fitted with 20 micron polypropylene bottom frit. To each reaction tube was added a solution of amine 4 (0.25 mmol) in dichloroethane (10 mL) and a 0.5 M dichloroethane solution of isocyanate 9 (0.1 mL, 0.5 mmol). The reaction mixture was agitated at room temperature for 16 h. To each reaction tube was added trisamine resin (Argonaut Tech. Inc., 1.5 mmol) and isocyanate resin (Argonaut Tech. Inc., 0.75 mmol). The reaction mixture was agitated at room temperature for 16 h. The reaction mixture was filtered and washed with a acetonitrile-dichloromethane solution (1:1 v/v, 2 mL). The filtrate was evaporated under reduced pressure and optionally purified by reversed phase HPLC to afford the desired urea 10.

### General Procedure for the Amination of 2-Chloro-5-Nitropyridine.

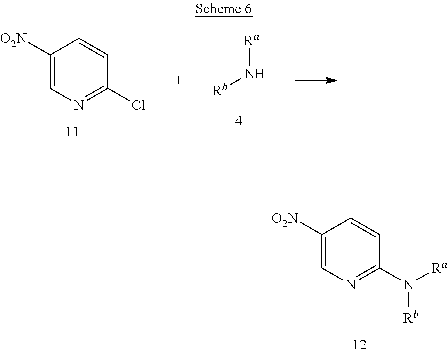

To a solution of 2-chloro-5-nitropyridine 11 (12.6 mmol) in dichloromethane (75 mL) was added amine 4 (25.3 mmol) and triethylamine (25.7 mmol). The reaction mixture was stirred at room temperature for 3 h. The organic solution was washed with water and brine. The organic layer was dried over anhydrous sodium sulfate. The organic solvent was evaporated under reduced pressure. The crude product was purified by flash column chromatography to yield the desired product 12.

### General Procedure for the Reduction of Nitro Compounds.

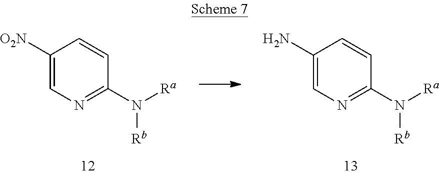

To a solution of the nitro compound 12 (12.0 mmol) dissolved in ethyl acetate (40 mL) and methanol (20 mL) was added palladium on charcoal (10% Pd, 1.2 g). The reaction mixture was agitated under a hydrogen atmosphere (45 psig) at room temperature for 3 h. The reaction mixture was filtered through Celite. The organic solvent was evaporated under reduced pressure yield the desired product 13.

### General Procedure for the Formation of Amide Compounds.

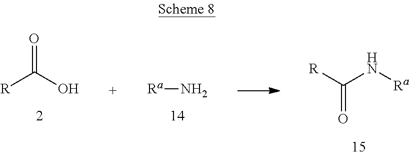

To a solution of the amine compound 14 (0.36 mmol) in dichloromethane (5 mL) was added carboxylic acid 2 (0.51 mmol), triethylamine (1.49 mmol) and Mukaiyama resin (Varian Polymer Lab., 1.0 mmol). The reaction mixture was agitated at room temperature for 1 h. Trisamine resin (Argonaut Tech. Inc., 1.0 mmol) was added and the reaction mixture was agitated at room temperature for 1 h. The reaction mixture was filtered and washed with dichloromethane. The organic solvent was evaporated under reduced pressure. The crude product was purified by flash column chromatography to yield the desired amide 15 were used.

### Example 1

### N-(6-(4-(methylsulfonyl)piperazin-1-yl)pyridin-3-yl)-4-phenyl-5-(trifluoromethyl)thiophene-2-carboxamide (19)

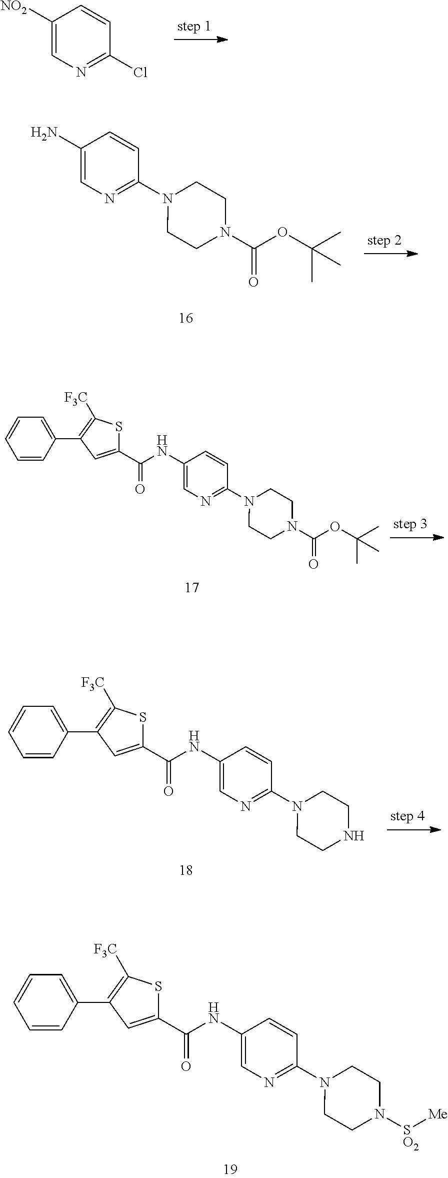

Step 1: tert-butyl 4-(5-aminopyridin-2-yl)piperazine-1-carboxylate (16)

Compound 16 was prepared using methods shown in Scheme 6 (wherein tert-butyl piperazine-1-carboxylate was used) and Scheme 7.

Step 2: tert-butyl 4-(5-(4-phenyl-5-(trifluoromethyl)thiophene-2-carboxamido)pyridin-2-yl)piperazine-1-carboxylate (17)

Compound 17 was prepared using method shown in Scheme 8 wherein tert-butyl 4-(5-aminopyridin-2-yl)piperazine-1-carboxylate (16) and 4-phenyl-5-(trifluoromethyl)thiophene-2-carboxylic acid were used. MS (M+1): 533.3

Step 3: 4-phenyl-N-(6-(piperazin-1-yl)pyridin-3-yl)-5-(trifluoromethyl)thiophene-2-carboxamide (18)

Compound 18 was prepared using method shown in Scheme 2 wherein tert-butyl 4-(5-(4-phenyl-5-(trifluoromethyl)thiophene-2-carboxamido)pyridin-2-yl)piperazine-1-carboxylate (17) was used.

Step 4: N-(6-(4-(methylsulfonyl)piperazin-1-yl)pyridin-3-yl)-4-phenyl-5-(trifluoromethyl)thiophene-2-carboxamide (19)

Compound 19 was prepared using method shown in Scheme 4 wherein 4-phenyl-N-(6-(piperazin-1-yl)pyridin-3-yl)-5-(trifluoromethyl)thiophene-2-carboxamide (18) and methanesulfonyl chloride were used.

### Example 2

### N-(6-(4-(methylsulfonyl)piperazin-1-yl)pyridin-3-yl)-2-phenyl-4-(trifluoromethyl)oxazole-5-carboxamide (20)

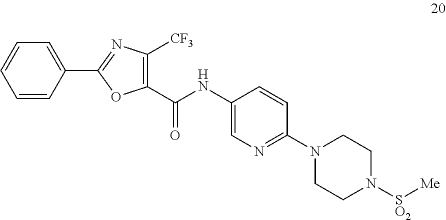

Compound 20 was prepared using the method for Example 1. MS (M+1): 496.3

### Example 3

### 6-(1,4-dioxa-8-azaspiro[4.5]decan-8-yl)pyridin-3-amine (21)

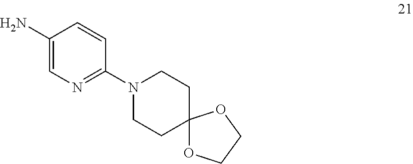

Compound 21 was prepared using methods shown in Scheme 6 (wherein 1,4-dioxa-8-azaspiro[4.5]decane was used) and Scheme 7.

### Example 4

### N-(6-(1,4-dioxa-8-azaspiro[4.5]decan-8-yl)pyridin-3-yl)-2-phenyl-4-(trifluoromethyl)oxazole-5-carboxamide (22)

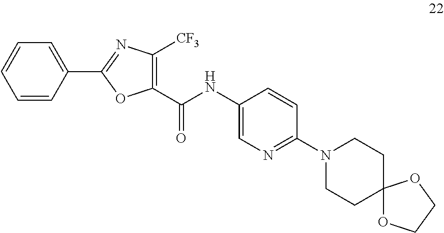

Compound 22 was prepared using method shown in Scheme 8 wherein 6-(1,4-dioxa-8-azaspiro[4.5]decan-8-yl)pyridin-3-amine (21) and 2-phenyl-4-(trifluoromethyl)oxazole-5-carboxylic acid were used. MS (M+1): 475.3

### Example 5

### N-(6-(1,4-dioxa-8-azaspiro[4.5]decan-8-yl)pyridin-3-yl)-4-phenyl-5-(trifluoromethyl)thiophene-2-carboxamide (23)

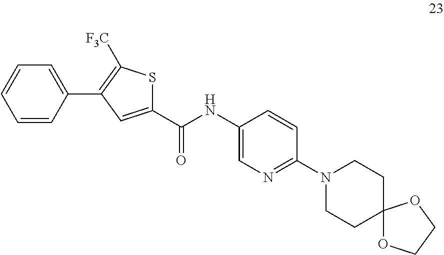

Compound 23 was prepared using method shown in Scheme 8 wherein 6-(1,4-dioxa-8-azaspiro[4.5]decan-8-yl)pyridin-3-amine (50) and 4-phenyl-5-(trifluoromethyl)thiophene-2-carboxylic acid were used. MS (M+1): 490.3

### Example 6

### N-(6-(1,4-dioxa-8-azaspiro[4.5]decan-8-yl)pyridin-3-yl)-2-methyl-1,5-diphenyl-1H-pyrrole-3-carboxamide (24)

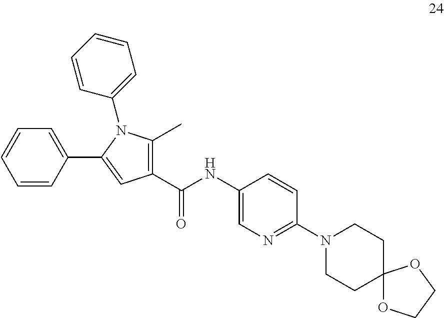

Compound 24 was prepared using method shown in Scheme 8 wherein 6-(1,4-dioxa-8-azaspiro[4.5]decan-8-yl)pyridin-3-amine (50) and 2-methyl-1,5-diphenyl-1H-pyrrole-3-carboxylic acid were used. MS (M+1): 495.3

### Example 7

### 4-Fluorophenyl 4-(5-(2-phenyl-5-(trifluoromethyl)oxazole-4-carboxamido)pyridin-2-yl)piperazine-1-carboxylate (27)

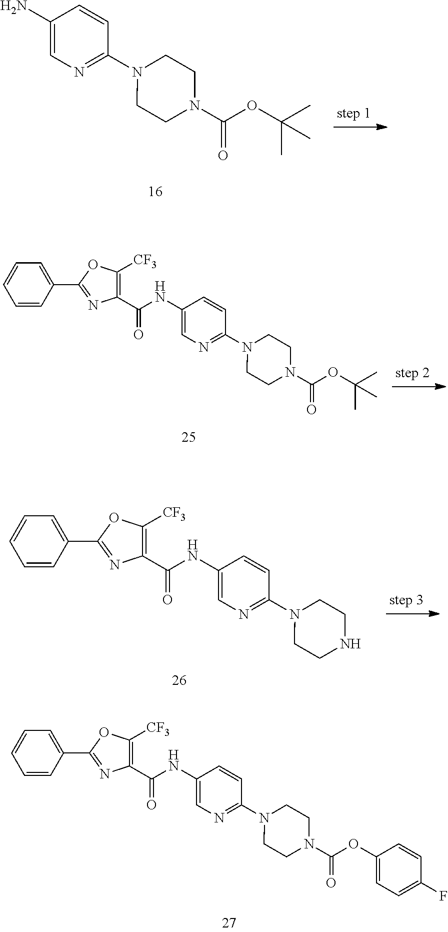

Step 1: tert-butyl 4-(5-(2-phenyl-5-(trifluoromethyl)oxazole-4-carboxamido)pyridin-2-yl)piperazine-1-carboxylate (25)

Compound 25 was prepared using method shown in Scheme 8 wherein tert-butyl 4-(5-aminopyridin-2-yl)piperazine-1-carboxylate (16) and 2-phenyl-5-(trifluoromethyl)oxazole-4-carboxylic acid were used. MS (M-4-1): 518.3

Step 2: 2-phenyl-N-(6-(piperazin-1-yl)pyridin-3-yl)-5-(trifluoromethyl)oxazole-4-carboxamide (26)

Compound 26 was prepared using method shown in Scheme 2 wherein tert-butyl 4-(5-(2-phenyl-5-(trifluoromethyl)oxazole-4-carboxamido)pyridin-2-yl)piperazine-1-carboxylate (25) was used.

Step 3: 4-Fluorophenyl 4-(5-(2-phenyl-5-(trifluoromethyl)oxazole-4-carboxamido)pyridin-2-yl)piperazine-1-carboxylate (27)

Compound 27 was prepared using method shown in Scheme 3 wherein 2-phenyl-N-(6-(piperazin-1-yl)pyridin-3-yl)-5-(trifluoromethyl)oxazole-4-carboxamide (26) and 4-fluorophenyl chloroformate were used. MS (M+1): 556.3 (55) and ethyl chloroformate were used. MS (M+1): 490.3

### Example 8

### 2-Fluoroethyl 4-(5-(2-phenyl-5-(trifluoromethyl)oxazole-4-carboxamido)pyridin-2-yl)piperazine-1-carboxylate (28)

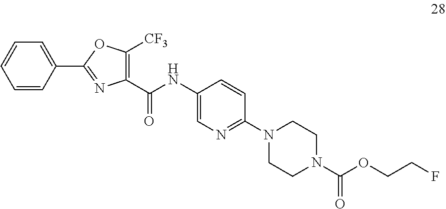

Compound 28 was prepared using method shown in Scheme 3 wherein 2-phenyl-N-(6-(piperazin-1-yl)pyridin-3-yl)-5-(trifluoromethyl)oxazole-4-carboxamide (26) and 2-fluoroethyl chloroformate were used. MS (M+1): 508.3

### Example 9

### But-2-ynyl 4-(5-(2-phenyl-5-(trifluoromethyl)oxazole-4-carboxamido)pyridin-2-yl)piperazine-1-carboxylate (29)

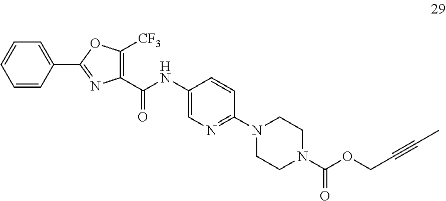

Compound 29 was prepared using method shown in Scheme 3 wherein 2-phenyl-N-(6-(piperazin-1-yl)pyridin-3-yl)-5-(trifluoromethyl)oxazole-4-carboxamide (26) and 2-butyn-1-yl chloroformate were used. MS (M+1): 514.3

### Example 10

### But-3-ynyl 4-(5-(2-phenyl-5-(trifluoromethyl)oxazole-4-carboxamido)pyridin-2-yl)piperazine-1-carboxylate (30)

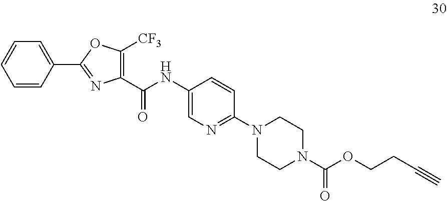

Compound 30 was prepared using method shown in Scheme 3 wherein 2-phenyl-N-(6-(piperazin-1-yl)pyridin-3-yl)-5-(trifluoromethyl)oxazole-4-carboxamide (26) and 3-butyn-1-yl chloroformate were used. MS (M+1): 514.3

### Example 11

### 2-Methoxyethyl 4-(5-(2-phenyl-5-(trifluoromethyl)oxazole-4-carboxamido)pyridin-2-yl)piperazine-1-carboxylate (31)

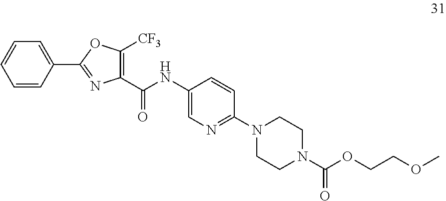

Compound 31 was prepared using method shown in Scheme 3 wherein 2-phenyl-N-(6-(piperazin-1-yl)pyridin-3-yl)-5-(trifluoromethyl)oxazole-4-carboxamide (26) and 2-methoxyethyl chloroformate were used. MS (M+1): 520.3

### Example 12

### Cyclopentyl 4-(5-(2-phenyl-5-(trifluoromethyl)oxazole-4-carboxamido)pyridin-2-yl)piperazine-1-carboxylate (32)

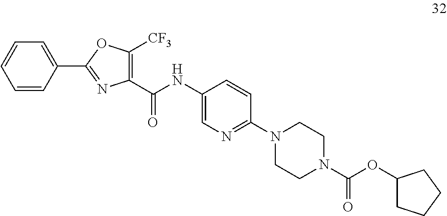

Compound 32 was prepared using method shown in Scheme 3 wherein 2-phenyl-N-(6-(piperazin-1-yl)pyridin-3-yl)-5-(trifluoromethyl)oxazole-4-carboxamide (26) and cyclopentyl chloroformate were used. MS (M+1): 530.3

### Example 13

### Phenyl 4-(5-(2-phenyl-5-(trifluoromethyl)oxazole-4-carboxamido)pyridin-2-yl)piperazine-1-carboxylate (33)

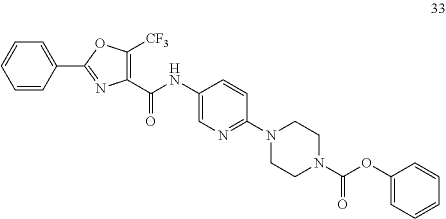

Compound 33 was prepared using method shown in Scheme 3 wherein 2-phenyl-N-(6-(piperazin-1-yl)pyridin-3-yl)-5-(trifluoromethyl)oxazole-4-carboxamide (26) and phenyl chloroformate were used. MS (M+1): 538.3

### Example 14

### p-Tolyl 4-(5-(2-phenyl-5-(trifluoromethyl)oxazole-4-carboxamido)pyridin-2-yl)piperazine-1-carboxylate (34)

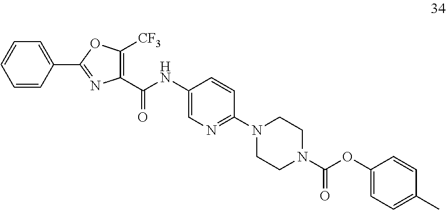

Compound 34 was prepared using method shown in Scheme 3 wherein 2-phenyl-N-(6-(piperazin-1-yl)pyridin-3-yl)-5-(trifluoromethyl)oxazole-4-carboxamide (55) and p-tolyl chloroformate were used. MS (M+1): 552.3

### Example 15

### 6-(4-phenylpiperazin-1-yl)pyridin-3-amine (35)

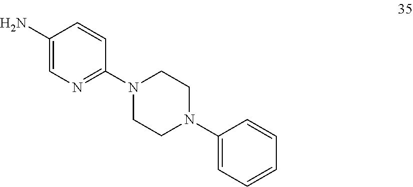

Compound 35 was prepared using methods shown in Scheme 6 (wherein 1-phenylpiperazine was used) and Scheme 7.

### Example 16

### 2-Phenyl-N-(6-(4-phenylpiperazin-1-yl)pyridin-3-yl)-5-(trifluoromethyl)oxazole-4-carboxamide (36)

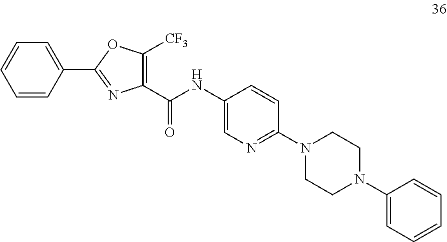

Compound 36 was prepared using method shown in Scheme 8 wherein 6-(4-phenylpiperazin-1-yl)pyridin-3-amine (35) and 2-phenyl-5-(trifluoromethyl)oxazole-4-carboxylic add were used. ¹H NMR (400 MHz, DMSO-d6) δ 10.54 (s, 1H), 8.54 (d, 1H, J=2.6 Hz), 8.15 (m, 2H), 7.99 (dd, 1H, J=9.2, 2.6 Hz), 7.62-7.70 (m, 3H), 7.24 (m, 2H), 7.00 (m, 2H), 6.97 (d, 1H, J=9.2 Hz), 6.81 (m, 1H), 3.63 (m, 4H), 3.25 (m, 4H). LCMS (ESI) Rt=3.83 min, [M+1]⁺=494.3.

### Example 17

### 6-(4-Phenylpiperidin-1-yl)pyridin-3-amine (37)

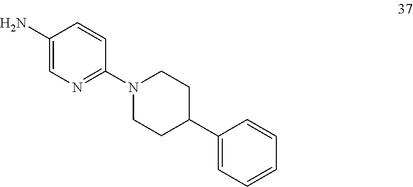

Compound 37 was prepared using methods shown in Scheme 6 (wherein 4-phenylpiperidine was used) and Scheme 7.

### Example 18

### 2-Phenyl-N-(6-(4-phenylpiperidin-1-yl)pyridin-3-yl)-5-(trifluoromethyl)oxazole-4-carboxamide (38)

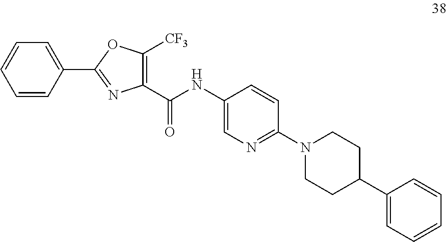

Compound 38 was prepared using method shown in Scheme 8 wherein 6-(4-phenylpiperidin-1-yl)pyridin-3-amine (37) and 2-phenyl-5-(trifluoromethyl)oxazole-4-carboxylic acid were used. ¹H NMR (400 MHz, DMSO-d6) δ 10.51 (s, 1H), 8.49 (d, 1H, J=2.6 Hz), 8.15 (m, 2H), 7.95 (dd, 1H, J=9.2, 2.9 Hz), 7.63-7.71 (m, 3H), 7.25-7.32 (m, 4H), 7.19 (m, 1H), 6.93 (d, 1H, J=9.2 Hz), 4.42 (m, 2H), 2.88 (m, 2H), 2.78 (m, 1H), 1.84 (m, 2H), 1.63 (m, 2H). LCMS (ESI) Rt=3.90 min, [M+1]⁺=493.3.

### Example 19

### N-(6-(4-(2-chlorophenylcarbamoyl)-1,4-diazepan-1-yl)pyridin-3-yl)-2-phenyl-5-(trifluoromethyl)oxazole-4-carboxamide (42)

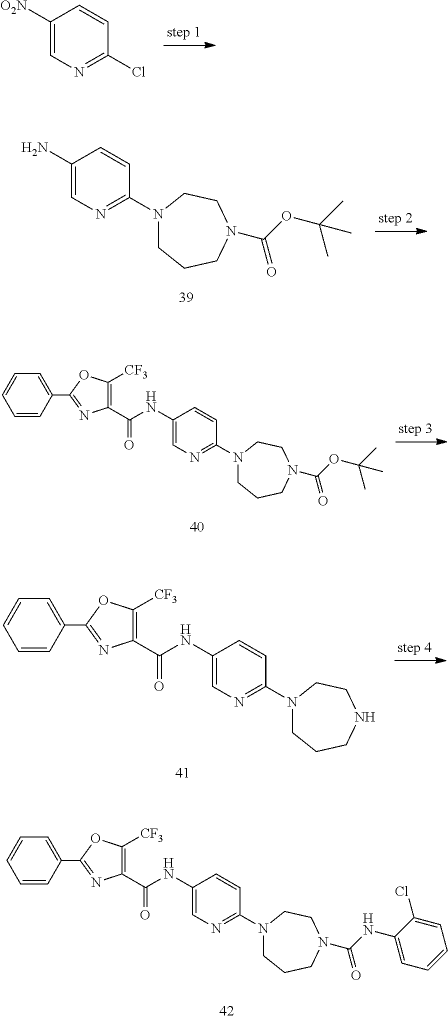

Step 1: tert-butyl 4-(5-aminopyridin-2-yl)-1,4-diazepane-1-carboxylate (39)

Compound 39 was prepared using methods shown in Scheme 6 (wherein tert-butyl 1,4-diazepane-1-carboxylate was used) and Scheme 7.

Step 2: tert-butyl 4-(5-(2-phenyl-5-(trifluoromethyl)oxazole-4-carboxamido)pyridin-2-yl)-1,4-diazepane-1-carboxylate (40)

Compound 40 was prepared using method shown in Scheme 8 wherein tert-butyl 4-(5-aminopyridin-2-yl)-1,4-diazepane-1-carboxylate (39) and 2-phenyl-5-(trifluoromethyl)oxazole-4-carboxylic acid were used, MS (M+1): 532.3

Step 3: N-(6-(1,4-diazepan-1-yl)pyridin-3-yl)-2-phenyl-5-(trifluoromethyl)oxazole-4-carboxamide (41)

Compound 41 was prepared using method shown in Scheme 2 wherein tert-butyl 4-(5-(2-phenyl-5-(trifluoromethyl)oxazole-4-carboxamido)pyridin-2-yl)-1,4-diazepane-1-carboxylate (40) was used.

Step 4: N-(6-(4-(2-chlorophenylcarbamoyl)-1,4-diazepan-1-yl)pyridin-3-yl)-2-phenyl-5-(trifluoromethyl)oxazole-4-carboxamide (42)

Compound 42 was prepared using method shown in Scheme 5 wherein N-(6-(1,4-diazepan-1-yl)pyridin-3-yl)-2-phenyl-5-(trifluoromethyl)oxazole-4-carboxamide (41) and 2-chlorophenyl isocyanate were used. MS (M+1): 585.3

### Example 20

### N-(6-(4-(2,6-dichlorophenylcarbamoyl)-1,4-diazepan-1-yl)pyridin-3-yl)-2-phenyl-5-(trifluoromethyl)oxazole-4-carboxamide (43)

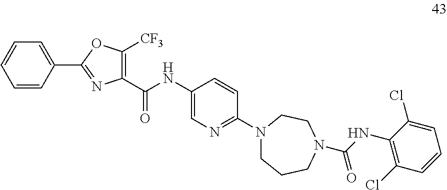

Compound 43 was prepared using method shown in Scheme 5 wherein N-(6-(1,4-diazepan-1-yl)pyridin-3-yl)-2-phenyl-5-(trifluoromethyl)oxazole-4-carboxamide (41) and 2,6-dichlorophenyl isocyanate were used. MS (M+1): 619.3

### Example 21

### N-(6-(4-(2,6-difluorophenylcarbamoyl)-1,4-diazepan-1-yl)pyridin-3-yl)-2-phenyl-5-(trifluoromethyl)oxazole-4-carboxamide (44)

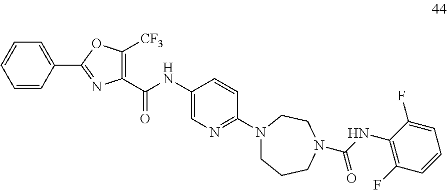

Compound 44 was prepared using method shown in Scheme 5 wherein N-(6-(1,4-diazepan-1-yl)pyridin-3-yl)-2-phenyl-5-(trifluoromethyl)oxazole-4-carboxamide (41) and 2,6-difluorophenyl isocyanate were used. MS (M+1): 587.3

### Example 22

### N-(6-(4-(2-fluorophenylcarbamoyl)-1,4-diazepan-1-yl)pyridin-3-yl)-2-phenyl-5-(trifluoromethyl)oxazole-4-carboxamide (45)

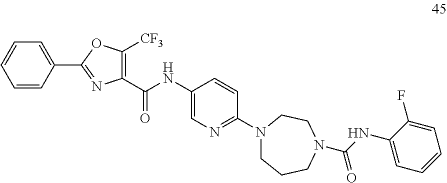

Compound 45 was prepared using method shown in Scheme 5 wherein N-(6-(1,4-diazepan-1-yl)pyridin-3-yl)-2-phenyl-5-(trifluoromethyl)oxazole-4-carboxamide (41) and 2-fluorophenyl isocyanate were used. MS (M+1): 569.3.

### Example 23

### N-(6-(5-(2-chlorophenylcarbamoyl)-2,5-diazabicyclo[2.2.1]heptan-2-yl)pyridin-3-yl)-2-phenyl-5-(trifluoromethyl)oxazole-4-carboxamide (49)

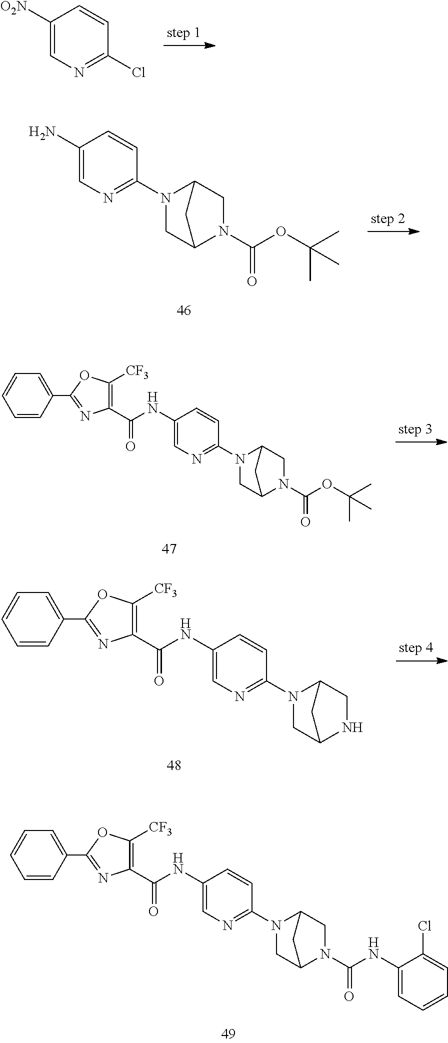

Step 1: tert-butyl 5-(5-aminopyridin-2-yl)-2,5-diazabicyclo[2.2.1]heptane-2-carboxylate (46)

Compound 46 was prepared using methods shown in Scheme 6 (wherein tert-butyl 2,5-diazabicyclo[2.2.1]heptane-2-carboxylate was used) and Scheme 7.

Step 2: tert-butyl 5-(5-(2-phenyl-5-(trifluoromethyl)oxazole-4-carboxamido)pyridin-2-yl)-2,5-diazabicyclo[2.2.1]heptane-2-carboxylate (47)

Compound 47 was prepared using method shown in Scheme 8 wherein tert-butyl 5-(5-aminopyridin-2-yl)-2,5-diazabicyclo[2.2.1]heptane-2-carboxylate (46) and 2-phenyl-5-(trifluoromethyl)oxazole-4-carboxylic acid were used. MS (M+1): 530.3

Step 3: N-(6-(2,5-diazabicyclo[2.2.1]heptan-2-yl)pyridin-3-yl)-2-phenyl-5-(trifluoromethyl)oxazole-4-carboxamide (48)

Compound 48 was prepared using method shown in Scheme 2 wherein tert-butyl 5-(5-(2-phenyl-5-(trifluoromethyl)oxazole-4-carboxamido)pyridin-2-yl)-2,5-diazabicyclo[2.2.1]heptane-2-carboxylate (47) was used.

Step 4: N-(6-(5-(2-chlorophenylcarbamoyl)-2,5-diazabicyclo[2.2.1]heptan-2-yl)pyridin-3-yl)-2-phenyl-5-(trifluoromethyl)oxazole-4-carboxamide (49)

Compound 49 was prepared using method shown in Scheme 5 wherein N-(6-(2,5-diazabicyclo[2.2.1]heptan-2-yl)pyridin-3-yl)-2-phenyl-5-(trifluoromethyl)oxazole-4-carboxamide (48) and 2-chlorophenyl isocyanate were used. MS (M+1): 583.3

### Example 24

### N-(6-(5-(2,6-dichlorophenylcarbamoyl)-2,5-diazabicyclo[2.2.1]heptan-2-yl)pyridin-3-yl)-2-phenyl-5-(trifluoromethyl)oxazole-4-carboxamide (50)

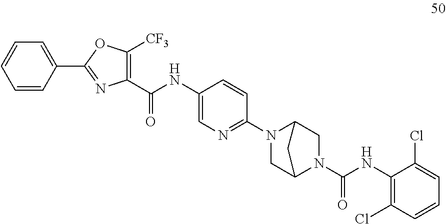

Compound 50 was prepared using method shown in Scheme 5 wherein N-(6-(2,5-diazabicyclo[2.2.1]heptan-2-yl)pyridin-3-yl)-2-phenyl-5-(trifluoromethyl)oxazole-4-carboxamide (48) and 2,6-dichlorophenyl isocyanate were used. MS (M+1): 617.3.

### Example 25

### N-(6-(5-(2,6-difluorophenylcarbamoyl)-2,5-diazabicyclo[2.2.1]heptan-2-yl)pyridin-3-0)-2-phenyl-5-(trifluoromethyl)oxazole-4-carboxamide (51)

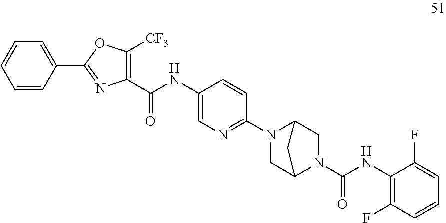

Compound 51 was prepared using method shown in Scheme 5 wherein N-(6-(2,5-diazabicyclo[2.2.1]heptan-2-yl)pyridin-3-yl)-2-phenyl-5-(trifluoromethyl)oxazole-4-carboxamide (48) and 2,6-difluorophenyl isocyanate were used. MS (M+1): 585.3.

### Example 26

### N-(6-(5-(2-fluorophenylcarbamoyl)-2,5-diazabicyclo[2.2.1]heptan-2-yl)pyridin-3-yl)-2-phenyl-5-(trifluoromethyl)oxazole-4-carboxamide (52)

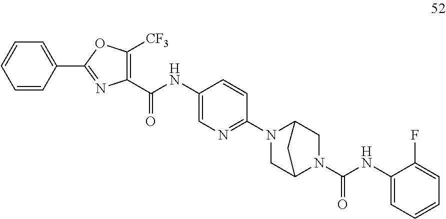

Compound 52 was prepared using method shown in Scheme 5 wherein N-(6-(2,5-diazabicyclo[2.2.1]heptan-2-yl)pyridin-3-yl)-2-phenyl-5-(trifluoromethyl)oxazole-4-carboxamide (48) and 2-fluorophenyl isocyanate were used. MS (M+1): 567.3.

### Example 27

### N-(6-(4-(3-chloropyridin-2-yl)piperazin-1-yl)pyridin-3-yl)-2-phenyl-4-(trifluoromethyl)oxazole-5-carboxamide (53)

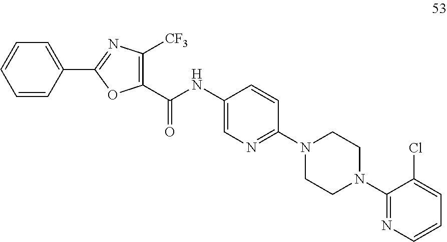

To a solution of 2-phenyl-N-(6-(piperazin-1-yl)pyridin-3-yl)-4-(trifluoromethyl)oxazole-5-carboxamide (15 mg, 0.036 mmol) dissolved in toluene (2 mL) and tetrahydrofuran (0.5 mL) was added 2,3-dichloropyridine (7.9 mg, 0.053 mmol), Pd₂ dba₃ (3.3 mg, 0.0036 mmol), BINAP (4.3 mg, 0.015 mmol) and sodium tert-butoxide (13.8 mg, 0.14 mmol). The reaction mixture was heated in a microwave reactor at 120° C. for 20 min. Dichloromethane and water was added. The organic layer was washed with water and brine. The organic layer was dried over anhydrous sodium sulfate. The organic solvent was evaporated under reduced pressure. The crude product was purified by flash column chromatography to yield the desired N-(6-(4-(3-chloropyridin-2-yl)piperazin-1-yl)pyridin-3-yl)-2-phenyl-4-(trifluoromethyl)oxazole-5-carboxamide (53) (9 mg, 0.017 mmol). MS (M+1): 529.3.

### Example 28

### N-(6-(4-(2-isobutyrylhydrazinecarbonyl)piperidin-1-yl)pyridin-3-yl)-2-phenyl-5-(trifluoromethyl)oxazole-4-carboxamide (54)

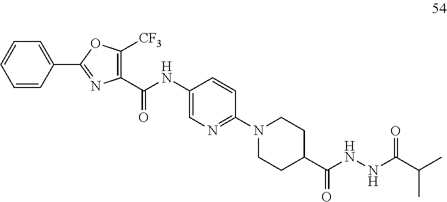

To a solution of 1-(5-(2-phenyl-5-(trifluoromethyl)oxazole-4-carboxamido)pyridin-2-yl)piperidine-4-carboxylic acid (46 mg, 0.10 mmol) in dichloroethane (2 mL) was added isobutyrohydrazide (15.3 mg, 0.15 mmol), triethylamine (31 mg, 0.31 mmol) and Mukaiyama resin (Varian Polymer Lab., 0.20 mmol). The reaction mixture was agitated at room temperature for 16 h. Trisamine resin (Argonaut Tech. Inc., 0.40 mmol) and isocyanate resin (Argonaut Tech. Inc.; 0.60 mmol) were added. The reaction mixture was agitated at room temperature for 16 h. The reaction mixture was filtered. The organic solvent was evaporated under reduced pressure to yield the desired N-(6-(4-(2-isobutyrylhydrazinecarbonyl)piperidin-1-yl)pyridin-3-yl)-2-phenyl-5-(trifluoromethyl)oxazole-4-carboxamide (54) (53 mg, 0.097 mmol) which was used in the next step without further purification.

### Example 29

### N-(6-(4-(5-isopropyl-1,3,4-oxadiazol-2-yl)piperidin-1-yl)pyridin-3-yl)-2-phenyl-5-(trifluoromethyl)oxazole-4-carboxamide (55)

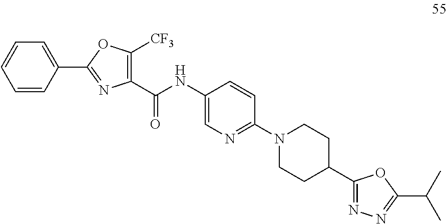

To a solution of N-(6-(4-(2-isobutyrylhydrazinecarbonyl)piperidin-1-yl)pyridin-3-yl)-2-phenyl-5-(trifluoromethyl)oxazole-4-carboxamide (53 mg, 0.097 mmol) in N,N-dimethylformamide (2 mL) was added tosyl chloride (23 mg, 0.12 mmol) and BEMP resin (Sigma-Aldrich, 0.50 mmol). The reaction mixture was heated in a microwave reactor at 200° C. for 30 min. The crude product was purified by reversed phase HPLC to yield the desired N-(6-(4-(5-isopropyl-1,3,4-oxadiazol-2-yl)piperidin-1-yl)pyridin-3-yl)-2-phenyl-5-(trifluoromethyl)oxazole-4-carboxamide (55) (3.7 mg, 0.007 mmol). MS (M+1): 527.3.

### Example 30

### N-(6-(4-(2-acetylhydrazinecarbonyl)piperidin-1-yl)pyridin-3-yl)-2-phenyl-5-(trifluoromethyl)oxazole-4-carboxamide (56)

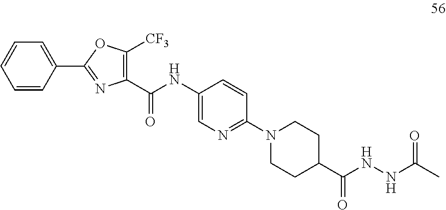

To a solution of 1-(5-(2-phenyl-5-(trifluoromethyl)oxazole-4-carboxamido)pyridin-2-yl)piperidine-4-carboxylic acid (92 mg, 0.20 mmol) in dichloromethane (8 mL) was added acetohydrazide (23 mg, 0.31 mmol), triethylamine (61 mg, 0.60 mmol) and Mukaiyama resin (Varian Polymer Lab., 0.40 mmol). The reaction mixture was agitated at room temperature for 16 h. Trisamine resin (Argonaut Tech. Inc., 0.80 mmol) and isocyanate resin (Argonaut Tech. Inc., 1.2 mmol) were added. The reaction mixture was agitated at room temperature for 16 h. The reaction mixture was filtered. The organic solvent was evaporated under reduced pressure to yield the desired N-(6-(4-(2-acetylhydrazinecarbonyl)piperidin-1-yl)pyridin-3-yl)-2-phenyl-5-(trifluoromethyl)oxazole-4-carboxamide (56) (78 mg, 0.15 mmol) which was used in the next step without further purification.

### Example 31

### N-(6-(4-(5-methyl-1,3,4-oxadiazol-2-yl)piperidin-1-yl)pyridin-3-yl)-2-phenyl-5-(trifluoromethyl)oxazole-4-carboxamide (57)

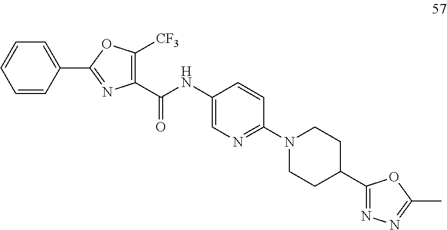

To a solution of N-(6-(4-(2-acetylhydrazinecarbonyl)piperidin-1-yl)pyridin-3-yl)-2-phenyl-5-(trifluoromethyl)oxazole-4-carboxamide (70 mg, 0.14 mmol) in N,N-dimethylformamide (5 mL) was added tosyl chloride (31 mg, 0.16 mmol) and BEMP resin (Sigma-Aldrich, 0.70 mmol). The reaction mixture was heated in a microwave reactor at 180° C. for 5 min. Diethyl ether and water was added. The organic layer was washed with water and brine. The organic layer was dried over anhydrous sodium sulfate. The organic solvent was evaporated under reduced pressure. The crude product was purified by flash column chromatography to yield the desired N-(6-(4-(5-methyl-1,3,4-oxadiazol-2-yl)piperidin-1-yl)pyridin-3-yl)-2-phenyl-5-(trifluoromethyl)oxazole-4-carboxamide (57) (18 mg, 0.036 mmol).

### Example 32

### 1-(5-(2-phenyl-4-(trifluoromethyl)oxazole-5-carboxamido)pyridin-2-yl)piperidin-4-yl dimethylphosphinate (58)

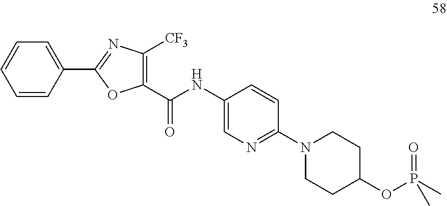

To a solution of N-(6-(4-hydroxypiperidin-1-yl)pyridin-3-yl)-2-phenyl-4-(trifluoromethyl)oxazole-5-carboxamide (35 mg, 0.081 mmol) dissolved in dichloromethane (2 mL) and N,N-dimethylformamide (0.5 mL) cooled in an ice bath was added dimethyl phosphinyl chloride (20.9 mg, 0.18 mmol) and triethylamine (16 mg, 0.16 mmol). The reaction mixture was stirred at 0° C. for 1 h. Dichloromethane and water was added. The organic layer was washed with saturated sodium bicarbonate and brine. The organic layer was dried over anhydrous sodium sulfate. The organic solvent was evaporated under reduced pressure. The crude product was purified by flash column chromatography to yield the desired 1-(5-(2-phenyl-4-(trifluoromethyl)oxazole-5-carboxamido)pyridin-2-yl)piperidin-4-yl dimethylphosphinate (58) (27 mg, 0.053 mmol). MS (M+1): 509.3

### Example 33

### N-(6-(4-benzyl-3-oxopiperazin-1-yl)pyridin-3-yl)-2-phenyl-4-(trifluoromethyl)oxazole-5-carboxamide (62)

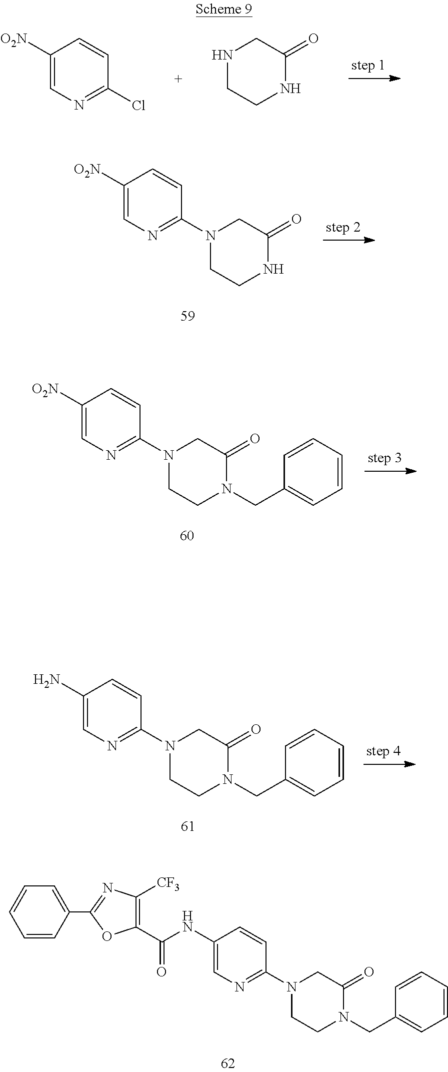

Step 1: 4-(5-nitropyridin-2-yl)piperazin-2-one (59)

To a solution of 2-chloro-5-nitropyridine (623 mg, 3.9 mmol) in DMF (6 mL) was added N,N-diisopropylethylamine (1.4 mL, 8.5 mmol) and 2-oxopiperazine (424 mg, 4.24 mmol). The reaction mixture was heated at 85° C. for 40 min by microwave. Then the reaction mixture was cooled to RT, and poured into water (150 mL). The precipitate was filtered, washed with water and dried under vacuum to yield 4-(5-nitropyridin-2-yl)piperazin-2-one (59) as a solid (588 mg, 68% yield). MS (M+1): 223.3

Step 2: 1-benzyl-4-(5-nitropyridin-2-yl)piperazin-2-one (60)

To a solution of 4-(5-nitropyridin-2-yl)piperazin-2-one (59) (65 mg, 1.64 mmol) in DMF (6 mL) was added, dropwise, 1.0 M sodium bis(trimethylsilyl)amide solution in THF (1.8 mL, 1.8 mmol), followed 1 min later by benzyl bromide (215 μL, 1.8 mmol), also dropwise. After 1 h, the reaction mixture was poured into water and extracted with EtOAc. The extract was dried over Na₂SO₄, filtered, and concentrated. The crude product was purified by silica gel chromatography (eluant: gradient of EtOAc in hexanes) to give 1-benzyl-4-(5-nitropyridin-2-yl)piperazin-2-one (60) (329 mg, 64% yield).

MS (M+1): 313.2

Step 3: 4-(5-aminopyridin-2-yl)-1-benzyl-piperazin-2-one (61)

The 1-benzyl-4-(5-nitropyridin-2-yl)piperazin-2-one (60) (195 mg, 0.63 mmol) was stirred in EtOAc (30 mL) and MeOH (15 mL). The mixture was treated with PtO₂ (72 mg) and stirred at RT under 1 atm of H₂ for 1 h 10 min. Then, the reaction mixture was filtered over a pad of celite and concentrated to yield 4-(5-aminopyridin-2-yl)-1-benzyl-piperazin-2-one (61) (176 mg, 100% yield).

MS (M+1): 283.2

Step 4: N-(6-(4-benzyl-3-oxopiperazin-1-yl)pyridin-3-yl)-2-phenyl-4-(trifluoromethyl)oxazole-5-carboxamide (62)

To a solution of 4-(5-aminopyridin-2-yl)-1-benzyl-piperazin-2-one (61) (176 mg, 0.625 mmol) in dry DMF (8 mL) was added 2-phenyl-4-(trifluoromethyl)oxazole-5-carboxylic acid (177 mg, 0.688 mmol), diisopropylethylamine (0.21 mL, 1.25 mmol), EDCI (80 mg, 0.938 mmol), and HOBT (127 mg, 0.938 mmol). The reaction mixture was stirred at room temperature for 16 h then poured into water and extracted with ethyl acetate. The combined organic extract was washed with saturated NaCl, dried (Na₂SO₄), filtered, and concentrated. Purification by silica gel chromatography (eluant: ethyl acetate-hexane gradient) gave N-(6-(4-benzyl-3-oxopiperazin-1-yl)pyridin-3-yl)-2-phenyl-4-(trifluoromethyl)oxazole-5-carboxamide (62) as a yellow solid (191 mg, 59% yield).

¹H NMR (500 MHz, CDCl₃) δ 8.37 (d, 1H, J=3 Hz), 8.20 (d, 2H, J=7 Hz), 8.10 (s, 1H), 8.08 (d, 1H, J=9 Hz), 7.65 (t, 1H, J=7.5 Hz), 7.60 (t, 2H, J=7 Hz), 7.40 (t, 2H, J=7 Hz), 7.35 (t, 2H, J=9 Hz), 6.65 (d, 1H, J=9 Hz), 4.73 (s, 2H), 4.25 (s, 2H), 3.90 (m, 2H), 3.45 (m, 2H). MS (M+1): 522.3

### Example 34

### N-(6-(3-Oxopiperazin-1-yl)pyridin-3-yl)-2-phenyl-4-(trifluoromethyl)oxazole-5-carboxamide (64)

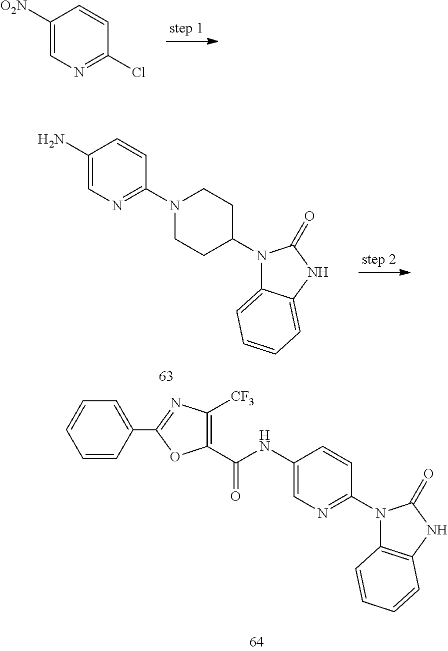

Step 1: 1-(1-(5-aminopyridin-2-yl)piperidin-4-yl)-1H-benzo[d]imidazo-2(3H)-one (63)

Intermediate 63 was prepared by the general procedures for step 1 and step 3 of intermediate 61, by using 2-chloro-5-nitropyridine and 1-(piperidin-4-yl)-1H-benzo[d]imidazo-2(3H)-one. MS (M+1): 310.2

Step 2: N-(6-(3-oxopiperazin-1-yl)pyridin-3-yl)-2-phenyl-4-(trifluoromethyl)oxazole-5-carboxamide (64)

Compound 64 was prepared by the general procedure for compound 62, by using 2-phenyl-4-(trifluoromethyl)oxazole-5-carboxylic acid and 1-(1-(5-aminopyridin-2-yl)piperidin-4-yl)-1H-benzo[d]imidazo-2(3H)-one (63). ¹H NMR (500 MHz, DMSO-d6) δ 10.70 (s, 1H), 8.47 (s, 1H), 8.27 (d, 2H, J=7 Hz), 8.10 (s, 1H), 7.92 (d, 1H, J=9 Hz), 7.65 (m, 3H), 6.92 (d, 1H, J=9 Hz), 4.00 (s, 2H), 3.75 (broad s, 2H), 3.30 (broad s, 2H). MS (M+1): 432.2

### Example 35

### N-(6-phenoxypyridin-3-yl)-2-phenyl-4-(trifluoromethyl)oxazole-5-carboxamide (66)

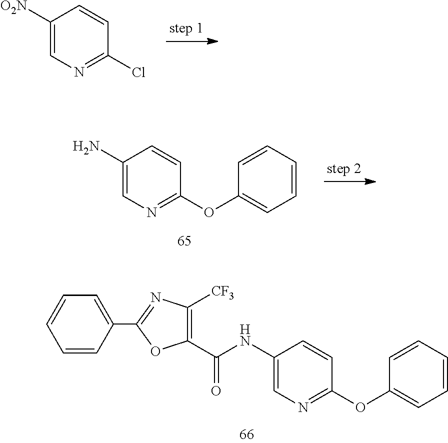

Step 1: 3-amino-5-phenoxy-pyridine (65)

Intermediate 65 was prepared by the general procedures for step 1 and step 3 of intermediate 61, by using 2-chloro-5-nitropyridine and phenol. MS (M+1): 187.2

Step 2: N-(6-phenoxypyridin-3-yl)-2-phenyl-4-(trifluoromethyl)oxazole-5-carboxamide (66)

Compound 66 was prepared by the general procedure for compound 62, by using 2-phenyl-4-(trifluoromethyl)oxazole-5-carboxylic acid and 3-amino-5-phenoxy-pyridine (65). ¹H NMR (500 MHz, CDCl₃) δ 8.33 (s, 1H), 8.32 (d, 1H, J=9 Hz), 8.20 (d, 2H, J=8 Hz), 8.10 (s, 1H), 7.65 (t, 1H, J=7.5 Hz), 7.60 (t, 2H, J=8 Hz), 7.45 (t, 2H, J=7.5 Hz), 7.27 (t, 1H, J=7.5 Hz), 7.18 (d, 2H, J=8.5 Hz), 7.02 (d, 1H, J=8.5 Hz). MS (M+1): 426.2

### Example 36

### N-(6-(4-methyl-3-oxopiperazin-1-yl)pyridin-3-yl)-2-phenyl-4-(trifluoromethyl)oxazole-5-carboxamide (68)

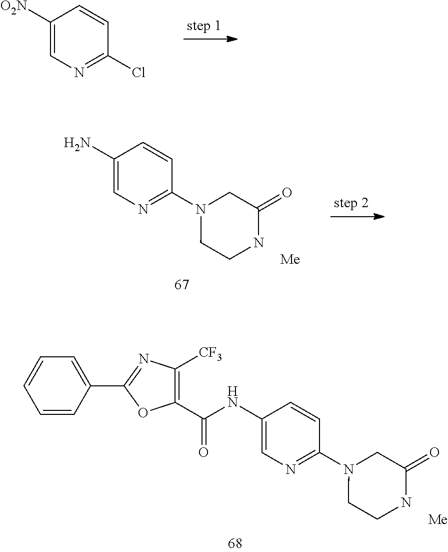

Step 1: 4-(5-aminopyridin-2-yl)-1-methyl-piperazin-2-one (67)

Intermediate 67 was prepared by the general procedure for intermediate 61, by using 2-chloro-5-nitropyridine, 2-oxopiperazine, and methyl iodide. MS (M+1): 207.1

Step 2: N-(6-(4-methyl-3-oxopiperazin-1-yl)pyridin-3-yl)-2-phenyl-4-(trifluoromethyl)oxazole-5-carboxamide (68)

Compound 67 was prepared by the general procedure for compound 62, by using 2-phenyl-4-(trifluoromethyl)oxazole-5-carboxylic acid and 4-(5-aminopyridin-2-yl)-1-methyl-piperazin-2-one (68). ¹H NMR (500 MHz, DMSO-d6) δ10.70 (s, 1H), 8.45 (s, 1H), 8.27 (d, 2H, J=6.5 Hz), 7.95 (d, 1H, J=9 Hz), 7.65 (m, 3H), 6.95 (d, 1H, J=8.5 Hz), 4.05 (s, 2H), 3.83 (t, 2H, J=6 Hz), 3.43 (t, 2H, J=5.5 Hz), 2.90 (s, 3H). MS (M-1-1): 446.2

### Example 37

### N-(6-(1-oxo-2,8-diazaspiro[4.5]decan-8-yl)pyridin-3-yl)-2-phenyl-4-(trifluoromethyl)oxazole-5-carboxamide (70)

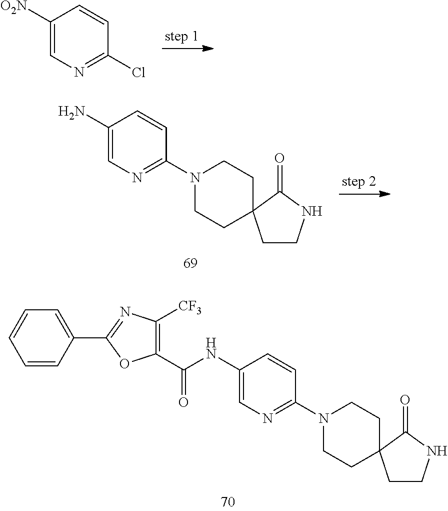

Step 1: 8-(5-aminopyridin-2-yl)-2,8-diazaspiro[4.5]decan-1-one (69)

Intermediate 69 was prepared by the general procedures for step 1 and step 3 of intermediate 61, by using 2-chloro-5-nitropyridine and 2,8-diazaspiro[4.5]decan-1-one. MS (M+1): 247.2

Step 2: N-(6-(1-oxo-2,8-diazaspiro[4.5]decan-8-yl)pyridin-3-yl)-2-phenyl-4-(trifluoromethyl)oxazole-5-carboxamide (70)

Compound 70 was prepared by the general procedure for compound 62, by using 2-phenyl-4-(trifluoromethyl)oxazole-5-carboxylic acid and 8-(5-aminopyridin-2-yl)-2,8-diazaspiro[4.5]decan-1-one (70). ¹H NMR (500 MHz, DMSO-d6) δ 10.67 (s, 1H), 8.43 (s, 1H), 8.27 (d, 2H, J=6.5 Hz), 7.87 (d, 1H, J=9 Hz), 7.67 (m, 3H), 7.60 (s, 1H), 6.93 (d, 1H, J=9 Hz), 4.18 (d, 2H, J=13 Hz), 3.22 (t, 2H, J=7 Hz), 3.03 (t, 2H, J=10.5 Hz), 2.05 (t, 2H, J=6.5 Hz), 1.67 (t, 2H, J=8.5 Hz), 1.40 (d, 2H, J=13.5 Hz). MS (M÷1): 486.3

### Example 38

### N-(6-(4-(2-oxo-2,3-dihydro-1H-benzo[d]imidazol-1-yl)piperidin-1-yl)pyridin-3-yl)-2-phenyl-4-(trifluoromethyl)oxazole-5-carboxamide (72)

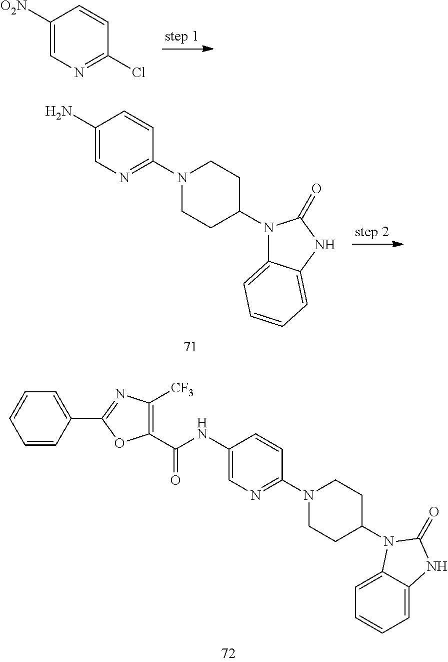

Step 1: 1-(1-(5-aminopyridin-2-yl)piperidin-4-yl)-1H-benzo[d]imidazo-2(3H)-one (71)

Intermediate 71 was prepared by the general procedures for step 1 and step 3 of intermediate 61, by using 2-chloro-5-nitropyridine and 1-(piperidin-4-yl)-1H-benzo[d]imidazo-2(3H)-one. MS (M+1): 310.2

Step 2: N-(6-(4-(2-oxo-2,3-dihydro-1H-benzo[d]imidazol-1-yl)piperidin-1-yl)pyridin-3-yl)-2-phenyl-4-(trifluoromethyl)oxazole-5-carboxamide (72)

Compound 72 was prepared by the general procedure for compound 62, by using 2-phenyl-4-(trifluoromethyl)oxazole-5-carboxylic acid and 1-(1-(5-aminopyridin-2-yl)piperidin-4-yl)-1H-benzo[d]imidazo-2(3H)-one (71). ¹H NMR (500 MHz, DMSO-d6) δ 10.85 (s, 1H), 10.70 (s, 1H), 8.45 (s, 1H), 8.27 (d, 2H, J=6.5 Hz), 7.93 (d, 1H, J=9 Hz), 7.67 (m, 3H), 7.13 (broad s, 1H), 7.00 (d, 1H, J=9.5 Hz), 6.97 (m, 3H), 4.48 (m, 3H), 3.00 (t, 2H, J=13 Hz), 2.35 (q, 2H, J=13 Hz), 1.77 (d, 2H, J=10.5 Hz). MS (M+1): 549.3.

### Example 39

### N-(6-(2,4-dibenzyl-3-oxopiperazin-1-yl)pyridin-3-yl)-2-phenyl-4-(trifluoromethyl)oxazole-5-carboxamide (74)

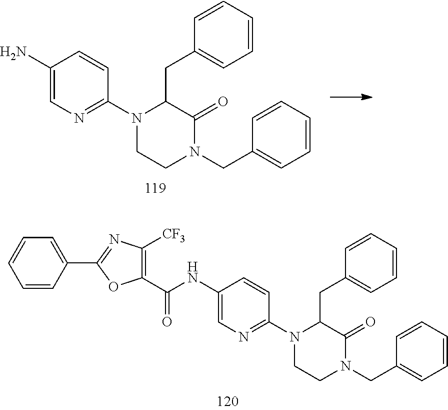

Compound 74 was prepared by the general procedure for compound 62, by using 2-phenyl-4-(trifluoromethyl)oxazole-5-carboxylic acid and 4-(5-aminopyridin-2-yl)-1,3-dibenzyl-piperazin-2-one which was prepared by dialkylation of intermediate 59 with benzyl bromide. ¹H NMR (500 MHz, CDCl₃) δ 8.32 (d, 1H, J=2.5 Hz), 8.20 (d, 2H, J=8 Hz), 8.03 (s, 1H), 7.93 (dd, 1H, J=2.5, 9 Hz), 7.65 (t, 1H, J=7 Hz), 7.60 (t, 2H, J=7.5 Hz), 7.35 (m, 3H), 7.25 (m, 5H), 7.20 (m, 2H), 6.38 (d, 1H, J=9.5 Hz), 5.00 (t, 1H, J=5.5 Hz), 4.87 (d, 1H, J=14.5 Hz), 4.45 (d, 1H, J=14.5 Hz), 4.13 (dt, 1H, J=13.5, 4 Hz), 3.47 (d, 2H, J−5 Hz), 3.33 (td, 1H, J=9, 4.5 Hz), 3.03 (td, 1H, J=9.5, 3.5 Hz), 2.82 (dt, 1H, J=12, 3.5 Hz). MS (M+1): 612.3.

### Example 40

### N-(6-chloropyridin-3-yl)-2-phenyl-4-(trifluoromethyl)oxazole-5-carboxamide (75)

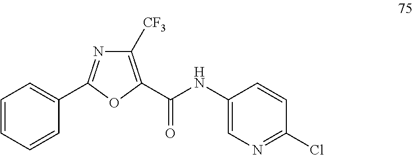

2-Phenyl-4-(trifluromethyl)oxazole-5-carboxylic acid (257 mg, 1 mmol) was added to a mixture of 6-chloropyridin-3-amine (257 mg, 2 mmol), HOBT (203 mg, 1.5 mmol), EDC (2.15 g, 3 eq at 1.39 mmol/g) in 15 mL of 1:3 CH₃CN:THF. The reaction mixture was stirred overnight at RT. Water (40 mL) was added, and the aqueous solution was extracted with 250 mL EtOAc. Organic extract was dried (Na₂SO₄), filtered, and concentrated. Purification by chromatography on an Analogix system (eluant: 1:1 EtOAc:hexane) to yield N-(6-chloropyridin-3-yl)-2-phenyl-4-(trifluoromethyl)oxazole-5-carboxamide (75) as white solid (0.48 g, 32% yield). ¹H NMR (500 MHz, CDCl₃) δ 7.37 to 7.39 (d, 1H), 7.52 to 7.63 (m, 3H), 8.10 to 8.15 (m, 3H), 8.31 to 8.34 (m, 1H), 8.50 to 8.51 (m, 1H).

### Examples 41-45

Compounds 76-80 were prepared by the combinatorial library synthesis described below:

To the corresponding 4-substituted piperidine (2 eq), Pd(dba)₂ (13 mg, 0.02 mmol), 2-(dicyclohexylphosphino)-2′-(N,N-dimethylamino)biphenyl (13 mg, 0.03 mmol), sodium t-butoxide (18 mg, 0.19 mmol) in toluene (2 mL) was added compound 75 (23 mg, 0.06 mmol). The reaction mixture was heated to 60° C. under nitrogen atmosphere overnight. To the reaction mixtures was added EtOAc (10 mL), washed with water (3 mL) and saturated NaCl solution (3 mL). The organic extract were dried (Na₂SO₄), filtered, and concentrated to give the crude products. The crude products were further purified by prep TLC using silica gel 2000 micron 20×20 cm plate developed in 2% MeOH in CH₂Cl₂ system to give the final products 76-80.

| STRUCTURE | LCMS (ESI) |
|---|---|
| 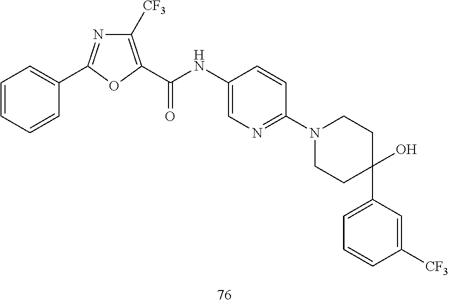 | Rt = 3.71 min, [M + 1]⁺ 577.3 |
| 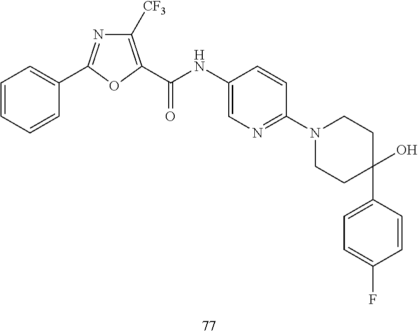 | Rt = 3.45 min, [M + 1]⁺ 527.3 |
| 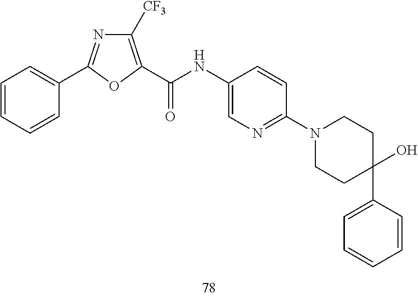 | Rt = 3.38 min, [M + 1]⁺ 509.3 |
| 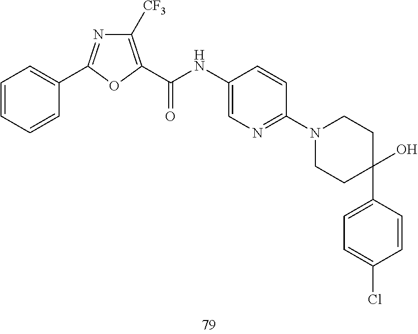 | Rt = 3.45 min, [M + 1]⁺ 527.3 |
| 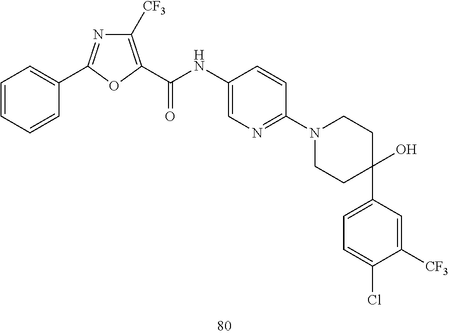 | Rt = 3.88 min, [M + 1]⁺ 611.3 |

### Example 46

### N-(6-Chloropyridin-3-yl)-2-phenyl-5-(trifluoromethyl)oxazole-4-carboxamide (81)

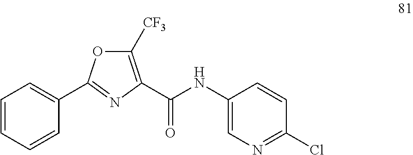

To a solution of 2-phenyl-5-(trifluoromethyl)oxazole-4-carboxylic acid (0.97 g, 3.77 mmol) and EDC (8.15 g, 3 eq at 1.39 mmol/g) in 55 mL of 3:1 CH₃CN:THF was added 6-chloropyridin-3-amine (0.97 g, 7.55 mmol) and HOBT (0.764, 5.66 mmol). The reaction mixture was stirred at RT overnight under nitrogen atmosphere. The reaction mixture was filtered and concentrated. Purification by chromatography on an Analogix system (eluant: 40% EtOAc in hexane) yielded N-(6-chloropyridin-3-yl)-2-phenyl-5-trifluoromethyloxazole-4-carboxamide (81) as white solid (0.37 g, 27% yield). MS (M+1): 368.

### Examples 47-49

Compounds 82-84 were prepared by the general procedures for compounds 75 and 76-80 by using the appropriate acids and 6-chloropyridin-3-amine as starting materials.

| STRUCTURE | LCMS (ESI) |
|---|---|
| 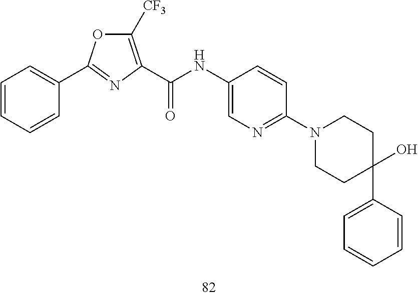 | Rt = 3.61 min, [M + 1]⁺ 509.3 |
| 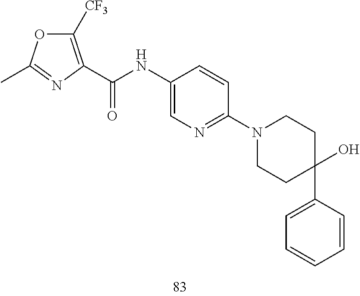 | Rt = 3.06 min, [M + 1]⁺ 447.2 |
| 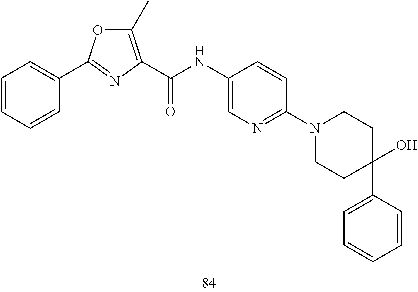 | Rt = 3.32 min, [M + 1]⁺ 455.3 |

### Example 50

### N-(6-(4-oxopiperidin-1-yl)pyridin-3-yl)-2-phenyl-5-(trifluoromethyl)oxazole-4-carboxamide (87)

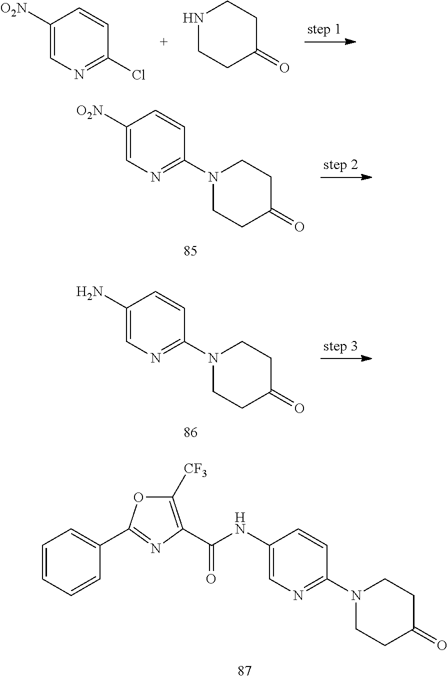

Step 1: 1-(5-Nitropyridin-2-yl)piperidin-4-one (85)

To a solution of 2-chloro-5-nitropyridine (0.5 g, 3.16 mmol) and pyridine-4-one hydrochloride (1.07 g, 6.33 mmol) in CH₂Cl₂ (12 mL) was added triethylamine (0.956 g, 9.46 mmol). The reaction mixture was stirred at 38° C. overnight. To the reaction mixture was added EtOAc (100 mL), washed with water (15 mL) and saturated NaCl solution (15 mL). The organic extract were dried (Na₂SO₄), filtered, and concentrated to give the crude product. Purification by chromatography on an Analogix system (eluant: 1:1 EtOAc:hexane) gave 1-(5-nitropyridin-2-yl)piperidin-4-one (85) as a yellow solid (0.63 g, 90% yield).

MS (M+1): 222.

Step 2: 1-(5-Aminopyridin-2-yl)piperidin-4-one (86)

To a solution of 85 (1 g, 4.52 mmol) and acetic acid (0.2 mL) in EtOAc (200 mL) under a nitrogen atmosphere was added palladium on activated carbon (Pd 10%, 100 mg). The resulting reaction mixture was stirred at −10° C. under hydrogen atmosphere (balloon) for 5 h. The catalyst was removed by filtration through celite and washed with 1:1 EtOAc:MeOH. The filtrate was concentrated to give the product 1-(5-aminopyridin-2-yl)piperidin-4-one (86) as a light yellow solid (0.74 g, 85% yield). MS (M+1): 192.

Step 3: N-(6-(4-oxopiperidin-1-yl)pyridin-3-yl)-2-phenyl-5-(trifluoromethyl)oxazole-4-carboxamide (87) 2-Phenyl-5-(trifluoromethyl)oxazole-4-carboxylic acid (300 mg, 1.17 mmol) was added to a mixture of compound 86 (450 mg, 2.34 mmol), HOBT (240 mg, 1.76 mmol), EDCI (560 mg, 2.93 mmol) in 70 mL of 1:3 CH₃CN:THF. The reaction mixture was stirred overnight at RT. Water (70 mL) was added, and the aqueous solution was extracted with 450 mL EtOAc. The organic extract was dried (Na₂SO₄), filtered, and concentrated. Purification by chromatography on an Analogix system (eluant: 40% EtOAc in hexane) gave N-(6-(4-oxopiperidin-1-yl)pyridine-3-yl)-2-phenyl-5-(trifluoromethyl)oxazole-4-carboxamide (87) as light yellow solid (0.22 g, 40% yield). MS (M+1): 431.

### Examples 51-54

Compounds 88-91 were prepared by the combinatorial library synthesis described below:

To a mixture of compound 87 (15 mg, 0.035 mmol, 1 eq) and CeCl₃ (13 mg, 0.0525 mmol, 1.5 eq) was added THF (2 mL). This mixture was cooled to −78° C. and 1.5 equivalent of each Grignard reagent in THF solution was slowly added under a nitrogen atmosphere followed by stirring at −78° C. for 30 min and then slowly warmed up to RT. The reaction mixture was stirred overnight at RT followed by quenching with water (2 mL). To the reaction mixture was added EtOAc (15 mL), washed with water (15 mL) and saturated NaCl solution (15 mL). The organic extract was dried (Na₂SO₄), filtered, and concentrated to give the crude products. The crude products were further purified by prep TLC using silica gel 2000 micron 20×20 cm plate (eluant: 2% MeOH in CH₂Cl₂) to give the final products 88-91.

| STRUCTURE | LCMS (ESI) |
|---|---|
| 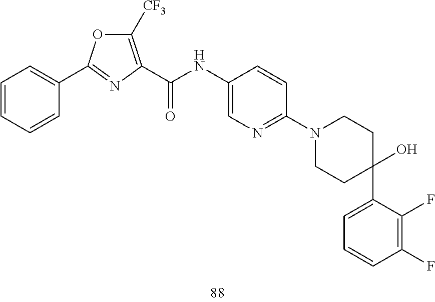 | Rt = 3.63 min, [M + 1]⁺ 545.3 |
| 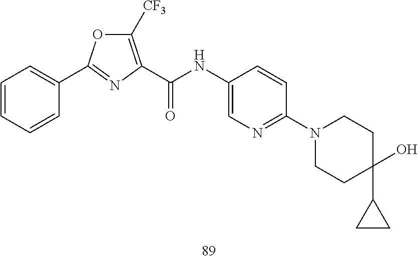 | Rt = 3.12 min, [M + 1]⁺ 473.3 |
| 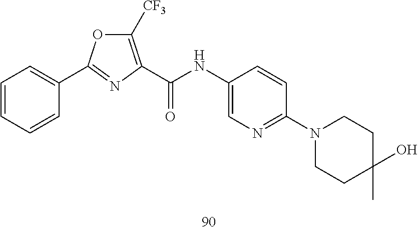 | Rt = 3.48 min, [M + 1]⁺ 447.2 |
| 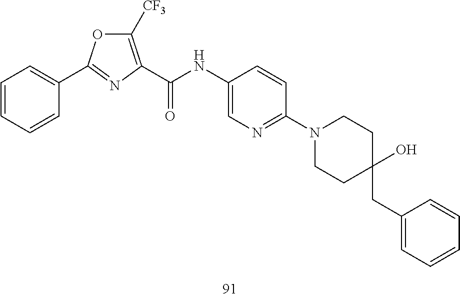 | Rt = 3.64 min, [M + 1]⁺ 523.3 |

### Example 55

### N-(6-(4-Cyano-4-phenylcyclohexyl)pyridin-3-yl)-2-phenyl-5-(trifluoromethyl)oxazole-4-carboxamide (92)

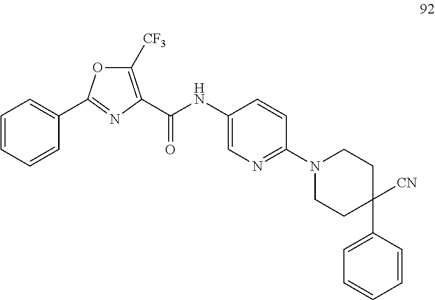

To 4-phenylpiperidine-4-carbonitrile (25 mg, 0.11 mmol), Pd(dba)₂ (11 mg, 0.02 mmol), 2-(dicyclohexylphosphino)-2′-(N,N-dimethylamino)biphenyl (11 mg, 0.03 mmol), sodium t-butoxide (20 mg, 0.19 mmol) in toluene (1.5 mL) was added compound 81 (20 mg, 0.05 mmol). The reaction mixture was heated to 65° C. under a nitrogen atmosphere overnight. To the reaction mixture was added EtOAc (15 mL), washed with water (2 mL) and saturated NaCl solution (3 mL). The organic extract was dried (Na₂SO₄), filtered, and concentrated to give the crude product. The crude product was purified by prep TLC using silica gel 2000 micron 20×20 cm plate (eluant: 2% MeOH in CH₂Cl₂) to give N-(6-(4-cyano-4-phenylpiperidin-1-yl)pyridine-3-yl)-2-phenyl-5-(trifluoromethyl)oxazole-4-carboxamide (92) as yellow solid (12.7 mg, 45% yield). ¹H NMR (500 MHz, CDCl₃) δ 2.06 to 2.14 (m, 2H), 2.19 to 2.13 (m, 2H), 3.30 to 3.38 (m, 2H), 4.43 to 4.47 (m, 2H), 6.74 to 6.77 (d, 1H), 7.34 to 7.36 (m, 1H), 7.39 to 7.43 (m, 2H), 7.47 to 7.59 (m, 5H), 7.92 (s, 1H), 8.04 to 8.07 (m, 1H), 8.13 to 8.16 (m, 2H), 8.27 to 8.28 (d, 1H); LCMS (ESI) Rt=4.14 min, [M+1]³⁰ 518.3.

### Example 56

### N-(6-(4-Fluoro-4-phenylcyclohexyl)pyridin-3-yl)-2-phenyl-5-(trifluoromethyl)oxazole-4-carboxamide (93)

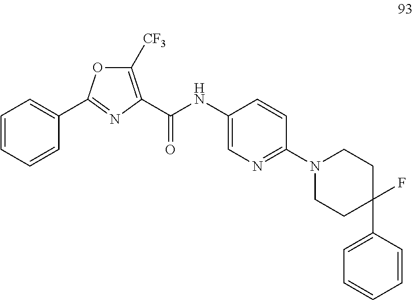

DAST (2 eq) was added to the solution of compound 82 (20 mg, 0.1 mmol) in THF (1.5 mL) at −78° C. The reaction mixture was stirred at −78° C. for 10 min then warmed up to 0° C. in 15 min. To the reaction mixture was added EtOAc (20 mL), washed with water (3 mL) and saturated NaCl solution (2 mL). The organic extract was dried (Na₂SO₄), filtered, and concentrated to give the crude product. To the crude product (25 mg) was added 3:1 DMF:H₂O (1 mL), OsO₄ (0.05 eq of 4 NH₂O solution), NMMNO (6 mg, 1.2 eq) followed by stirring at RT for 4 h. To the reaction mixture was added EtOAc (20 mL), washed with water (3 mL) and saturated NaCl solution (2 mL). The organic extract was dried (Na₂SO₄), filtered, and concentrated to give the crude product. Purification by prep TLC using silica gel 2000 micron 20×20 cm plate (eluant: 1:2 EtOAc:hexane) gave N-(6-(4-fluoro-4-phenylpiperidin-1-yl)pyridine-3-yl)-2-phenyl-5-(trifluoromethyl)oxazole-4-carboxamide (93). (9.2 mg) ¹H NMR (500 MHz, CDCl₃) δ 2.02 to 2.21 (m, 4H), 3.30 to 3.37 (m, 2H), 4.26 to 4.31 (m, 2H), 6.76 to 6.78 (d, 2H), 7.28 to 7.39 (m, 5H), 7.52 to 7.61 (m, 3H), 8.12 to 8.18 (m, 3H), 8.29 to 8.30 (d, 1H), 8.78 (s, 1H); LCMS (ESI) Rt=4.34 min, [M+1]⁺ 511.3.

### Examples 57-104

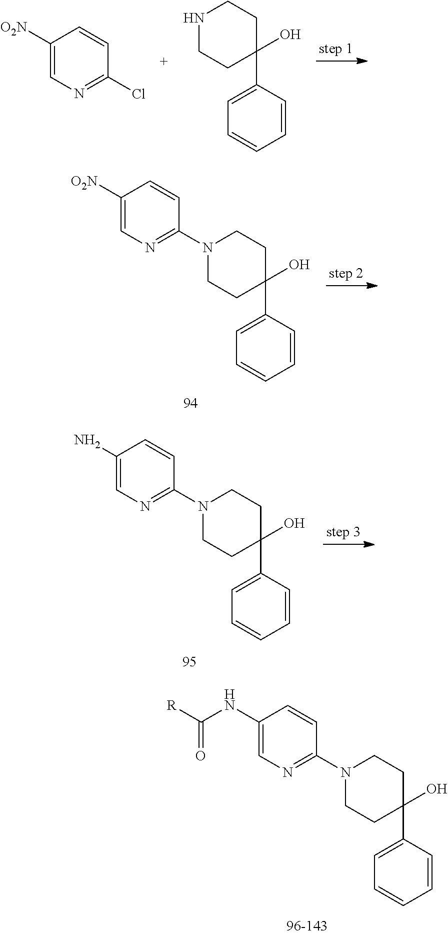

Step 1: 1-(5-nitropyridin-2-yl)-4-hydroxy-4-phenylpiperidine (94)

To a solution of 2-chloro-5-nitropyridine (0.9 g) in EtOH (5 mL) was added N,N-diisopropylethylamine (2.9 mL) and 4-hydroxy-4-phenylpiperidine (1.5 g). The reaction mixture was heated at 140° C. for 20 min by microwave. The reaction mixture was cooled to RT, diluted with EtOAc (150 mL), washed with H₂O (2×100 mL), saturated NH₄Cl (3×100 mL), brine (1×100 mL), dried over Na₂SO₄, filtered, and concentrated to give compound 94.

Step 2: 1-(5-aminopyridin-2-O-4-hydroxy-4-phenylpiperidine (95)

Compound 94 was suspended in EtOAc (50 mL) and EtOH (50 mL). The suspension was treated with 5% Pd/C (0.5 g) and stirred at RT under 1 atmosphere of H₂ for 16 h. The catalyst was removed by filtration over a pad of celite and concentrated to yield 1-(5-aminopyridin-2-yl)-4-hydroxy-4-phenylpiperidine (95) as a purple solid (1.0 g, 69% yield). LCMS (ESI) calcd for [M+1]⁺ 270.2. found 270.1.

Compounds 96-143 were prepared by the amide combinatorial library synthesis described below:

Step 3: The following reactions were run in 8 mL vials. To each vial was added EDCI (35.5 mg, 0.19 mmol), 20 mg of compound 95 (0.07 mmol) and HOBT (15 mg, 0.11 mmol), 2 mL of 3:1 CH₃CN:THF, and 0.11 mmol of each carboxylic acid. The vials were shaken overnight. To the reaction mixture was added EtOAc (10 mL), washed with water (3 mL) and saturated NaCl solution (3 mL). The organic extract waw dried (Na₂SO₄), filtered, and concentrated to give the crude product. The crude product was purified by prep TLC using silica gel 2000 micron 20×20 cm plate (eluant: 1:2 EtOAc:hexane) to give the final amide products (96-143) as below:

| STRUCTURE | LCMS (ESI) |
|---|---|
| 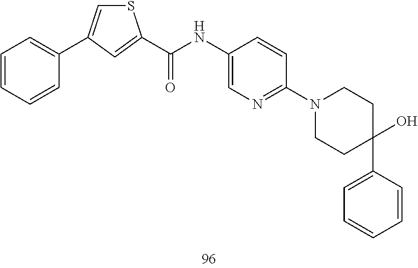 | Rt = 3.36 min, [M + 1]⁺ 456.3 |
| 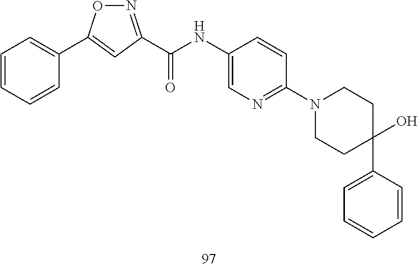 | Rt = 3.25 min, [M + 1]⁺ 441.2 |
| 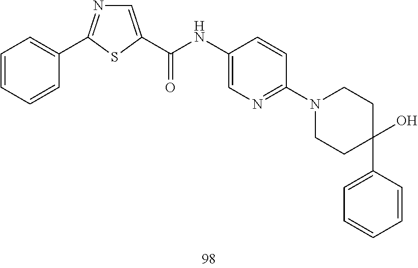 | Rt = 3.30 min, [M + 1]⁺ 457.3 |
| 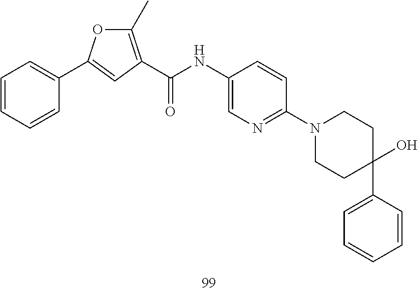 | Rt = 3.46 min, [M + 1]⁺ 454.2 |
| 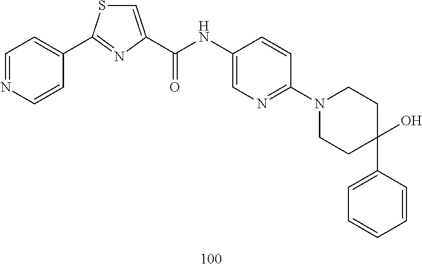 | Rt = 2.21 min, [M + 1]⁺ 458.2 |
| 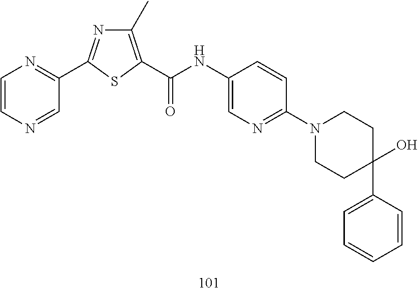 | Rt = 2.77 min, [M + 1]⁺ 473.3 |
| 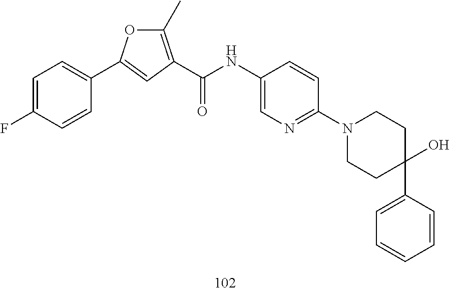 | Rt = 3.57 min, [M + 1]⁺ 472.3 |
| 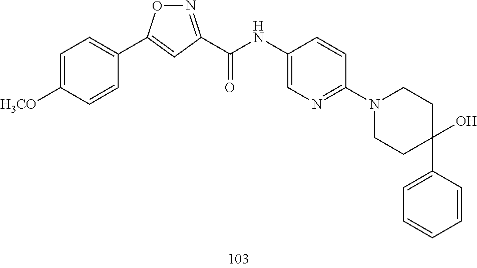 | Rt = 3.33 min, [M + 1]⁺ 471.3 |
| 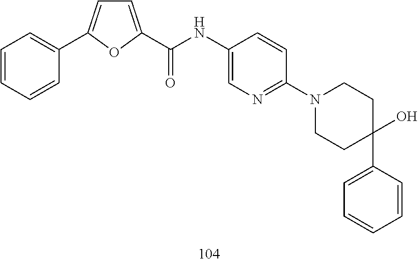 | Rt = 3.33 min, [M + 1]⁺ 440.2 |
| 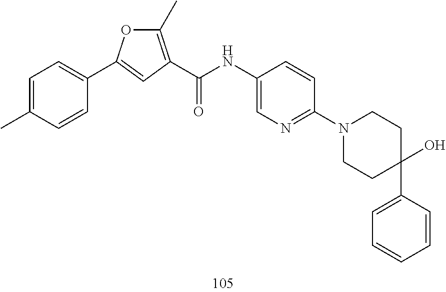 | Rt = 3.47 min, [M + 1]⁺ 468.3 |
| 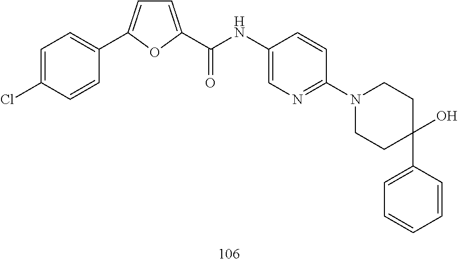 | Rt = 3.30 min, [M + 1]⁺ 474.3 |
| 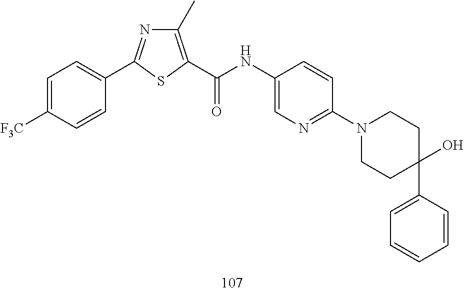 | Rt = 3.48 min, [M + 1]⁺ 539.3 |
| 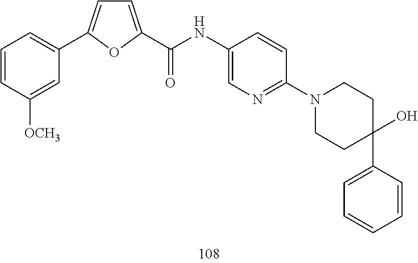 | Rt = 3.11 min, [M + 1]⁺ 470.3 |
| 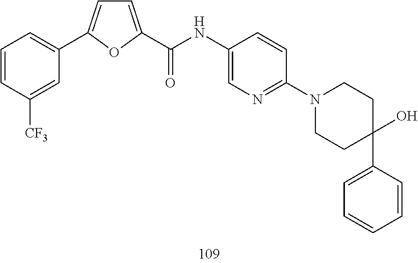 | Rt = 3.39 min, [M + 1]⁺ 508.3 |
| 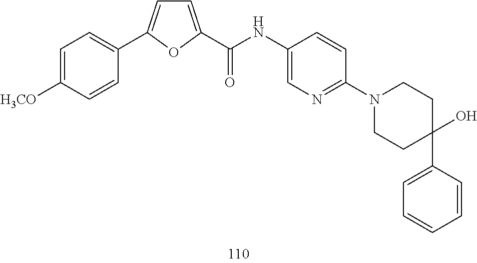 | Rt = 3.05 min, [M + 1]⁺ 470.3 |
| 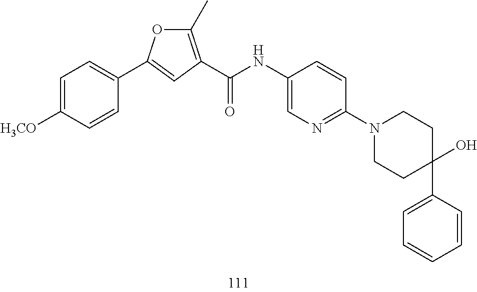 | Rt = 3.28 min, [M + 1]⁺ 484.3 |
| 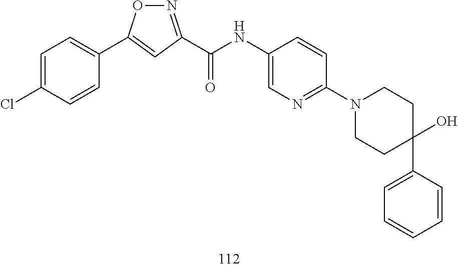 | Rt = 3.44 min, [M + 1]⁺ 475.3 |
| 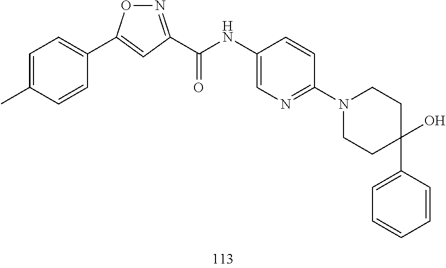 | Rt = 3.39 min, [M + 1]⁺ 455.3 |
| 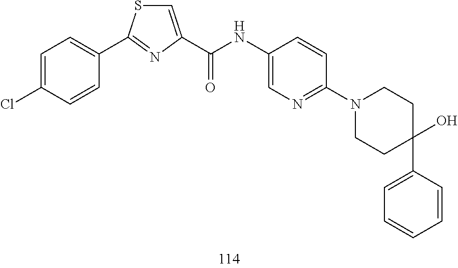 | Rt = 3.50 min, [M + 1]⁺ 491.3 |
| 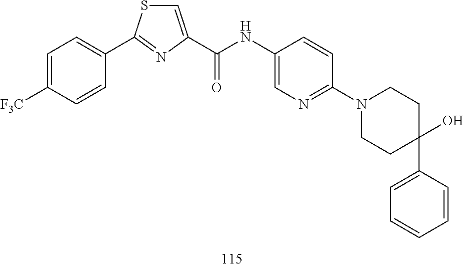 | Rt = 3.45 min, [M + 1]⁺ 525.3 |
| 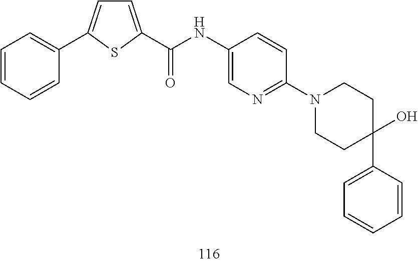 | Rt = 3.20 min, [M + 1]⁺ 456.3 |
| 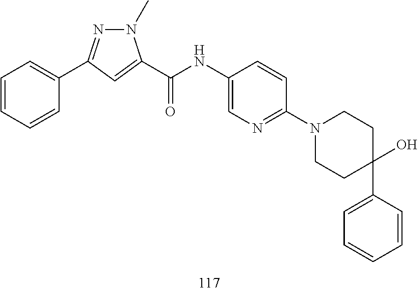 | Rt = 2.95 min, [M + 1]⁺ 454.2 |
| 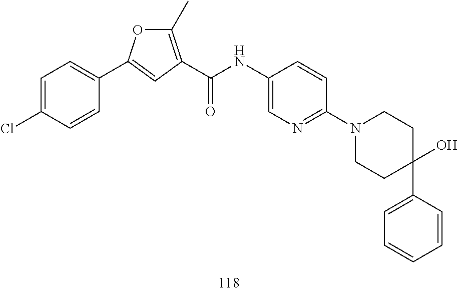 | Rt = 3.66 min, [M + 1]⁺ 488.3 |
| 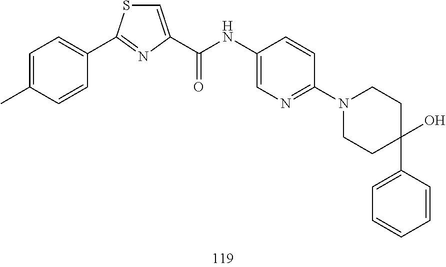 | Rt = 3.43 min, [M + 1]⁺ 471.3 |
| 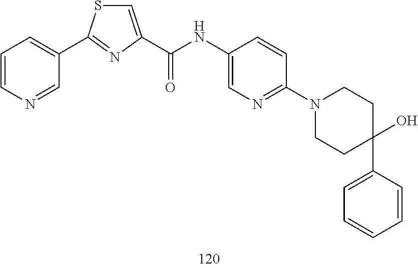 | Rt = 2.32 min, [M + 1]⁺ 458.3 |
| 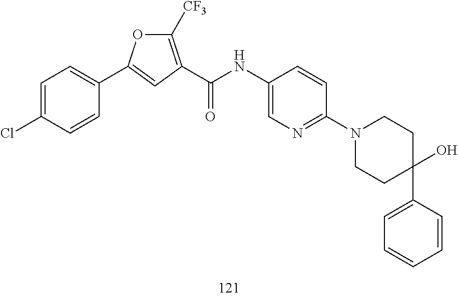 | Rt = 3.79 min, [M + 1]⁺ 542.3 |
| 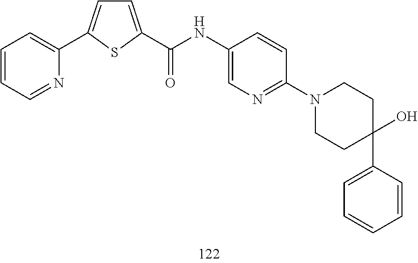 | Rt = 2.79 min, [M + 1]⁺ 457.3 |
| 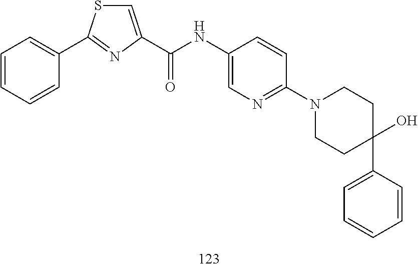 | Rt = 3.25 min, [M + 1]⁺ 457.3 |
| 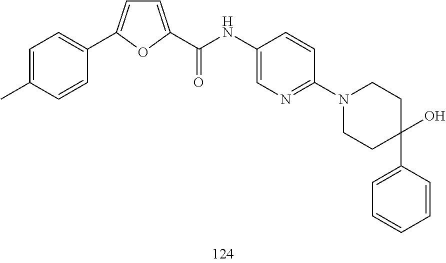 | Rt = 3.36 min, [M + 1]⁺ 454.2 |
| 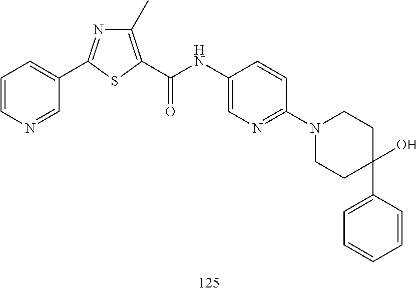 | Rt = 2.35 min, [M + 1]⁺ 472.3 |
| 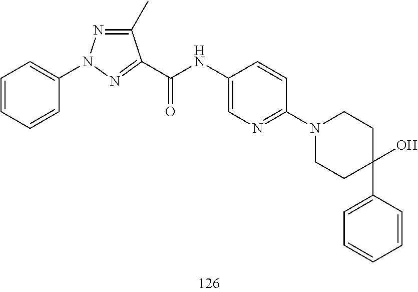 | Rt = 3.31 min, [M + 1]⁺ 455.3 |
| 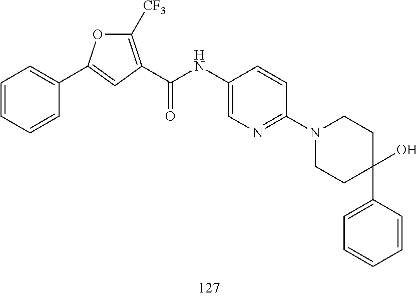 | Rt = 3.50 min, [M + 1]⁺ 508.3 |
| 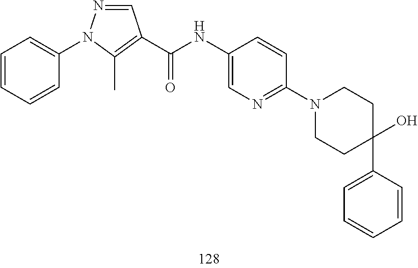 | Rt = 2.95 min, [M + 1]⁺ 454.2 |
| 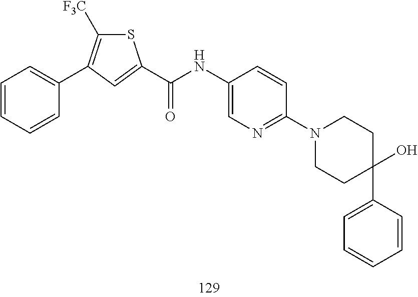 | Rt = 3.78 min, [M + 1]⁺ 524.3 |
| 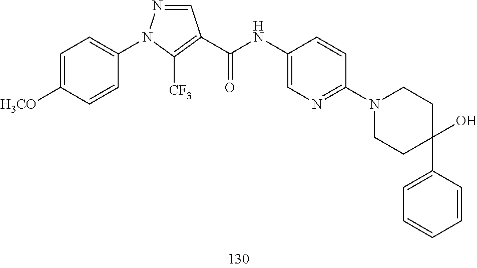 | Rt = 3.33 min, [M + 1]⁺ 538.3 |
| 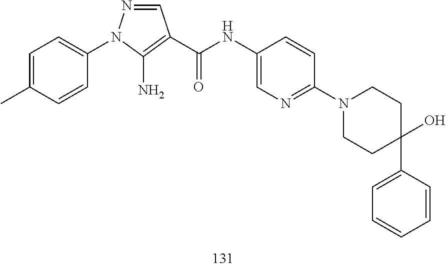 | Rt = 2.80 min, [M + 1]⁺ 469.3 |
| 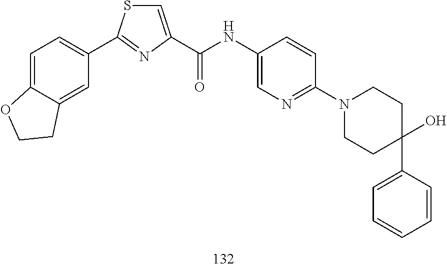 | Rt = 3.18 min, [M + 1]⁺ 499.3 |
| 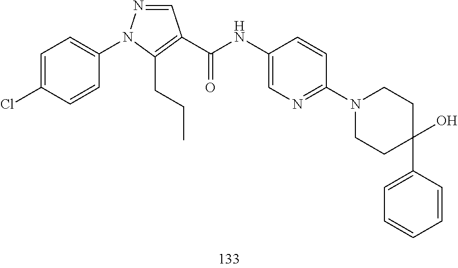 | Rt = 3.33 min, [M + 1]⁺ 516.3 |
| 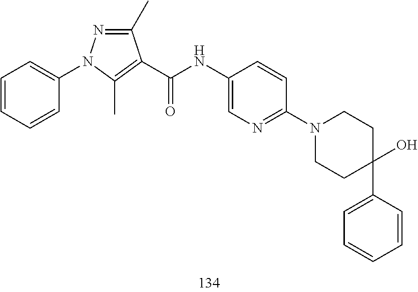 | Rt = 2.67 min, [M + 1]⁺ 468.3 |
| 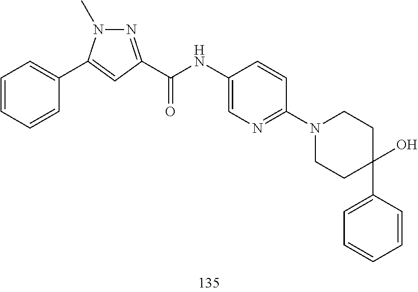 | Rt = 2.88 min, [M + 1]⁺ 454.2 |
| 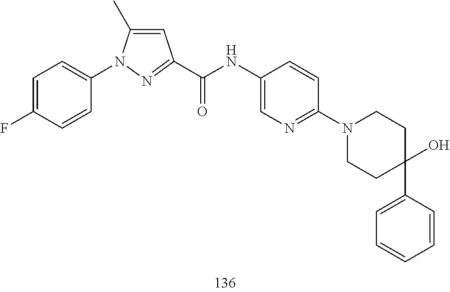 | Rt = 1.97 min, [M + 1]⁺ 472.3 |
| 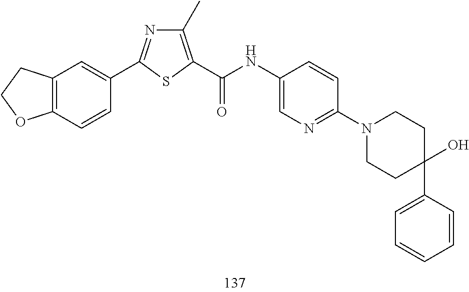 | Rt = 3.00 min, [M + 1]⁺ 513.3 |
| 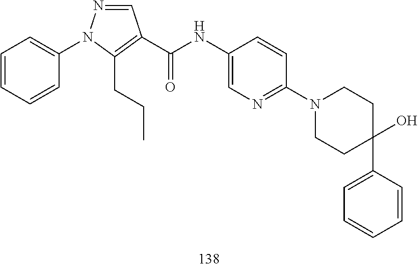 | Rt = 3.00 min, [M + 1]⁺ 482.3 |
| 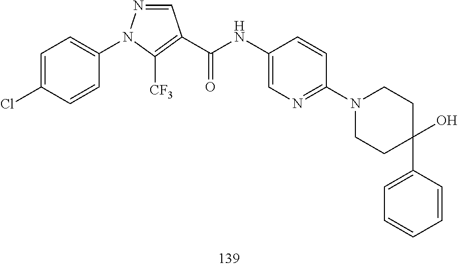 | Rt = 3.24 min, [M + 1]⁺ 542.3 |
| 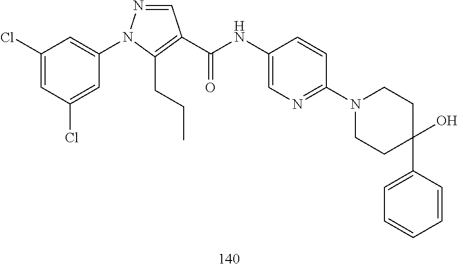 | Rt = 3.53 min, [M + 1]⁺ 550.3 |
| 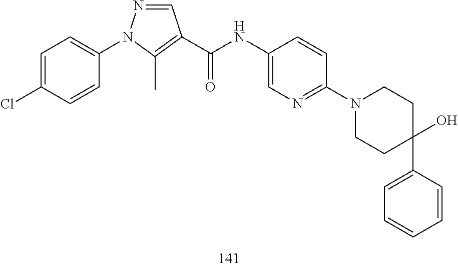 | Rt = 3.09 min, [M + 1]⁺ 488.3 |
| 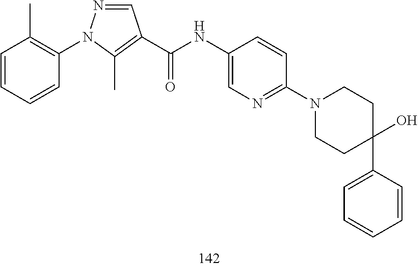 | Rt = 2.94 min, [M + 1]⁺ 468.3 |
| 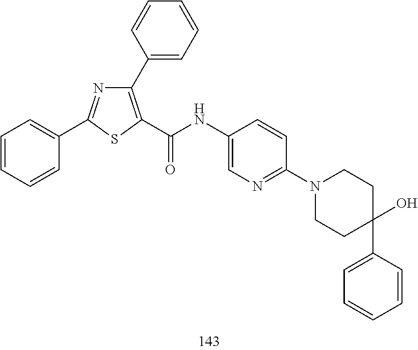 | Rt = 3.60 min, [M + 1]⁺ 533.3 |

### Example 105

### 2-Phenyl-N-[6-(4-(N-phenylsulfamoyl)piperidin-1-yl)pyridin-3-yl]-5-(trifluoromethyl)oxazole-4-carboxamide (194)

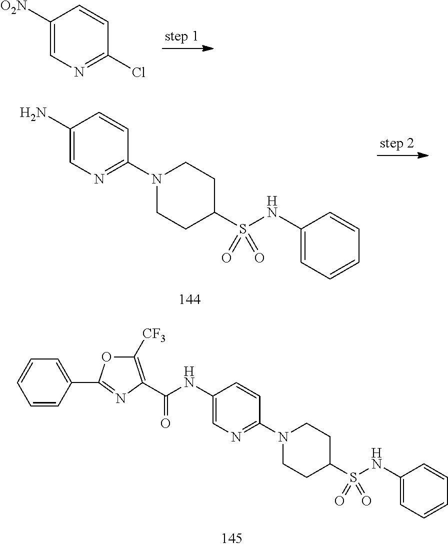

Step 1: 6-[4-(N-phenylsulfamoyl)piperidin-1-yl]pyridin-3-amine (144)

Intermediate 144 was prepared by the general procedures for step 1 and step 3 of intermediate 61, by using 2-chloro-5-nitropyridine and 4-(N-phenylsulfamoyl)-piperidine as starting materials, MS (M+1): 334.

Step 2: 2-phenyl-N-[6-(4-(N-phenylsulfamoyl)piperidin-1-yl)pyridin-3-yl]-5-(trifluoromethyl)oxazole-4-carboxamide (145)

Compound 145 was prepared by the general procedure for compound 62, by using 2-phenyl-5-(trifluoromethyl)oxazole-4-carboxylic acid and intermediate 144 as starting materials. ¹H NMR (500 MHz, CDCl₃) δ 8.81 (s, 1H), 8.32 (s, 1H), 8.11-8.13 (m, 2H), 7.35-7.25 (m, 4H), 7.16-7.11 (m, 2H), 6.68 (d, 1H, J=9.1 Hz), 4.42-4.12 (m, 2H), 3.28 (m, 1H), 2.81 (m, 2H), 2.19-2.16 (m, 2H), 1.97-1.88 (m, 2H), MS (M+1): 572.

### Example 106

### 2-Phenyl-N-[6-(4-(N-phenylsulfamoyl)piperidin-1-yl)pyridin-3-yl]-4-(trifluoromethyl)oxazole-5-carboxamide (146)

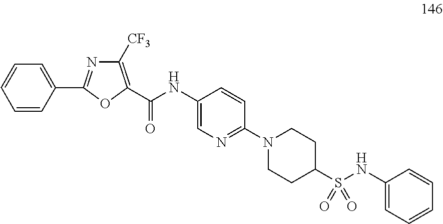

Compound 146 was prepared by the general procedure for compound 62, by using 2-phenyl-4-(trifluoromethyl)oxazole-5-carboxylic acid and intermediate 144 as starting materials. ¹H NMR (500 MHz, DMSO-d6) δ 10.67 (s, 1H), 9.87 (s, 1H), 8.42 (s, 1H), 8.42 (m, 1H), 8.26 (m, 2H), 7.88 (m, 1H), 7.69-7.64 (m, 3H), 7.34-7.23 (m, 4H), 7.09 (m, 1H), 6.94 (d, 1H, J=9.1 Hz), 4.37 (d, 2H, J=12.9 Hz), 3.41-3.37 (m, 2H), 2.86 (m, 2H), 2.01 (m, 2H), 1.67-1.59 (m, 2H).

MS (M+1): 572.

### Example 107

### N-[6-(4-(N-benzylsulfamoyl)piperidin-1-yl)pyridin-3-yl]-2-phenyl-5-(trifluoromethyl)oxazole-4-carboxamide (148)

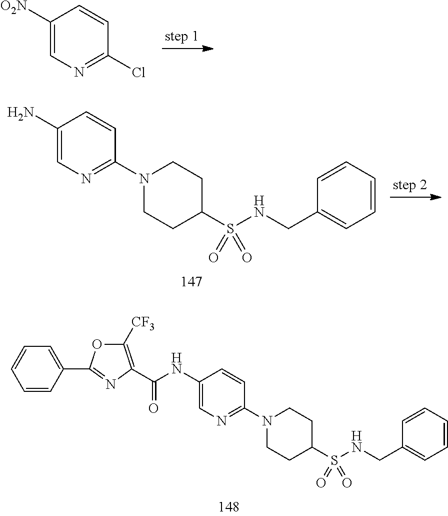

Step 1: 6-(4-(N-benzylsulfamoyl)piperidin-1-yl)pyridin-3-amine (147)

Intermediate 147 was prepared by the general procedures for step 1 and step 3 of intermediate 61, by using 2-chloro-5-nitropyridine and 4-(N-benzylsulfamoyl)piperidine as starting materials. MS (M+1): 347.

Step 2: N-[6-(4-(N-benzylsulfamoyl)piperidin-1-yl)pyridin-3-yl]-2-phenyl-5-(trifluoromethyl)oxazole-4-carboxamide (148)

Compound 148 was prepared by the general procedure for compound 62, by using 2-phenyl-5-(trifluoromethyl)oxazole-4-carboxylic acid and intermediate 147 as starting materials. ¹H NMR (500 MHz, CDCl₃) δ 9.02 (s, 1H), 8.47-8.45 (m, 2H), 8.21-8.19 (m, 2H), 7.66-7.59 (m, 3H), 7.43-7.37 (m, 8H), 6.87 (d, 1H, J=9.1 Hz), 4.70 (m, 1H), 4.38 (m, 4H), 3.04 (m, 1H), 2.27-2.24 (m, 2H), 1.96-1.94 (m, 2H). MS (M+1): 586.

### Example 108

### N-[6-(4-(N-benzylsulfamoyl)piperidin-1-yl)pyridin-3-yl]-2-phenyl-4-(trifluoromethyl)oxazole-5-carboxamide (149)

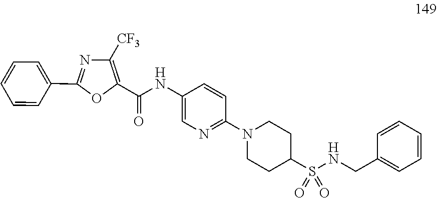

Compound 149 was prepared by the general procedure for compound 62, by using 2-phenyl-4-(trifluoromethyl)oxazole-5-carboxylic acid and intermediate 147 as starting materials. ¹H NMR (500 MHz, CDCl₃) δ 9.53 (s, 1H), 8.49 (s, 1H), 8.35-8.34 (m, 1H), 8.23-8.21 (m, 2H), 7.59-7.52 (m, 3H), 7.40-7.35 (m, 7H), 6.83 (d, 1H, J=9.1 Hz), 5.06 (m, 1H), 4.36-4.30 (m, 4H), 3.0-2.95 (m, 3H), 2.21-2.288 (m, 2H), 1.92-1.85 (m, 2H). MS (M+1): 586.

### Example 109

### N-[6-(4-Phenethylpiperidin-1-yl)pyridin-3-yl]-2-phenyl-4-(trifluoromethyl)oxazole-5-carboxamide (151)

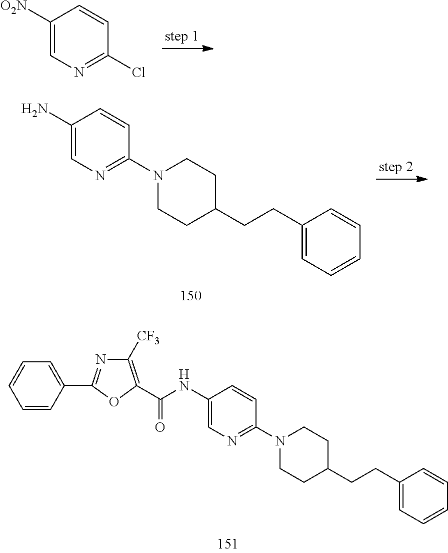

Step 1: 6-(4-phenethylpiperidin-1-yl)pyridin-3-amine (150)

Intermediate 150 was prepared by the general procedures for step 1 and step 3 of intermediate 61, by using 2-chloro-5-nitropyridine and 4-phenethylpiperidine as starting materials. MS (M+1): 312.

Step 2: N-[6-(4-phenethylpiperidin-1-yl)pyridin-3-yl]-2-phenyl-4-(trifluoromethyl)oxazole-5-carboxamide (151)

Compound 151 was prepared by the general procedure for compound 62, by using 2-phenyl-5-(trifluoromethyl)oxazole-4-carboxylic acid and intermediate 150 as starting materials. ¹H NMR (500 MHz, CDCl₃) δ 6.24 (d, 1H, J=2.8 Hz), 8.18 (m, 2H), 8.03-8.01 (dd, 11-1, J=3 Hz, J=9 Hz), 7.89 (s, 1H), 7.64-7.56 (m, 3H), 7.33-7.28 (m, 7H), 7.21 (m, 4H), 6.71 (d, 1H, J=9.1 Hz), 4.29 (d, 1H, J=12.9 Hz), 2.88-2.83 (m, 2H), 2.71-2.68 (m, 2H), 1.87 (m, 2H), 1.65-1.61 (m, 2H), 1.35-1.27 (m, 2H). MS (M+1): 521.

### Example 110

### N-[6-(4-(Benzyloxy)piperidin-1-yl)pyridin-3-yl]-2-phenyl-4-(trifluoromethyl)oxazole-5-carboxamide (153)

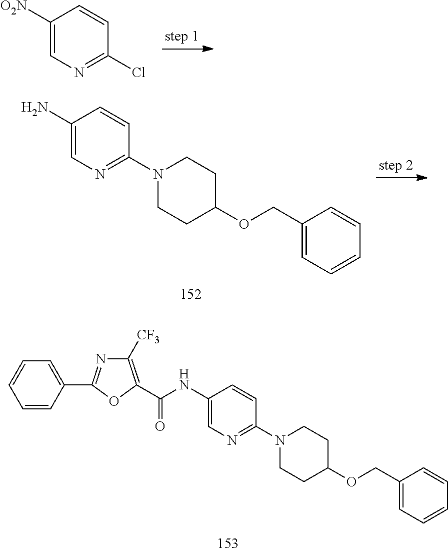

Step 1: 6-(4-(benzyloxy)piperidin-1-yl)pyridin-3-amine (152)

Intermediate 152 was prepared by the general procedures for step 1 and step 3 of intermediate 61, by using 2-chloro-5-nitropyridine and 4-benzyloxypiperidine as starting materials. MS (M+1): 284.

Step 2: N-[6-(4-(benzyloxy)piperidin-1-yl)pyridin-3-yl]-2-phenyl-4-(trifluoromethyl)oxazole-5-carboxamide (153)

Compound 153 was prepared by the general procedure for compound 62, by using 2-phenyl-4-(trifluoromethyl)oxazole-5-carboxylic acid and intermediate 152 as starting materials. ¹H NMR (500 MHz, CDCl₃) 8.25 (d, 1H, J=3 Hz), 8.16-8315 (m, 2H), 8.0 (m, 2H), 7.62-7.54 (m, 3H), 7.40-7.29 (m, 5H), 6.71 (d, 1H, 9.1 Hz), 4.61 (s, 2H), 4.02-3.97 (m, 2H), 3.71-3.66 (m, 1H), 3.29-3.24 (m, 2H), 2.03-1.99 (m, 2H), 1.77-1.70 (m, 2H). MS (M+1): 523.

### Example 111

### N-[6-(4-(Hydroxy(phenyl)methyl)piperidin-1-yl)pyridin-3-yl]-2-phenyl-4-(trifluoromethyl)oxazole-5-carboxamide (155)

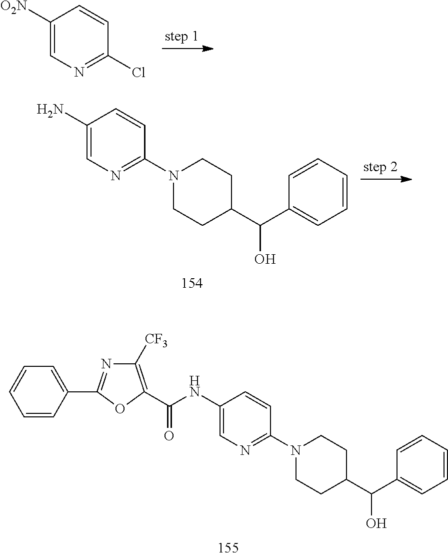

Step 1: 6-(4-(hydroxy(phenyl)methyl)piperidin-1-yl)pyridin-3-amine (154)

Intermediate 154 was prepared by the general procedures for step 1 and step 3 of intermediate 61, by using 2-chloro-5-nitropyridine and 4-(hydroxyphenylmethyl)piperidine as starting materials. MS (M+1): 284.

Step 2: N-[6-(4-(hydroxy(phenyl)methyl)piperidin-1-yl)pyridin-3-yl]-2-phenyl-4-(trifluoromethyl)oxazole-5-carboxamide (155)

Compound 155 was prepared by the general procedure for compound 62, by using 2-phenyl-4-(trifluoromethyl)oxazole-5-carboxylic acid and intermediate 154 as starting materials. ¹H NMR (500 MHz, CDCl₃) δ 8.22 (d, 1H, J=2.4 Hz), 8.15 (m, 1H), 8.02-7.97 (m, 1H), 7.62-7.53 (m, 2H), 7.40-7.30 (m, 3H), 6.67 (d, 1H, J=9.1 Hz), 4.41 (d, 1H, J=7 Hz), 4.36-4.23 (m, 1H), 2.86-2.72 (m, 1H), 2.12 (m, 1H), 1.88 (m, 1H), 1.47-1.25 (m, 2H). MS (M+1): 523.

### Example 112

### N-[6-(4-benzyl-5-oxo-1,4-diazepan-1-yl)pyridin-3-yl]-2-phenyl-4-(trifluoromethyl)oxazole-5-carboxamide (157)

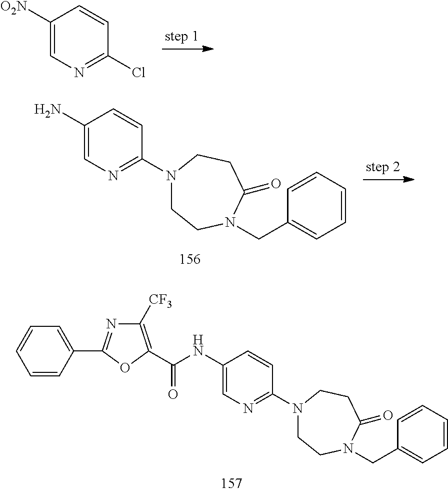

Step 1: 6-(4-benzyl-5-oxo-1H-1,4-diazepan-1-yl)pyridin-3-amine (156)

Intermediate 156 was prepared by the general procedures for step 1 and step 3 of intermediate 61, by using 2-chloro-5-nitropyridine and hexahydro-5-oxo-4-(phenylmethyl)-1H-1,4-diazepine as starting materials. MS (M+1): 297.

Step 2: N-[6-(4-benzyl-5-oxo-1,4-diazepan-1-yl)pyridin-3-yl]-2-phenyl-4-(trifluoromethyl)oxazole-5-carboxamide (157)

Compound 157 was prepared by the general procedure for compound 62, by using 2-phenyl-4-(trifluoromethyl)oxazole-5-carboxylic acid and intermediate 156 as starting materials. ¹H NMR (500 MHz, CDCl₃) δ 8.25 (m, 1H), 8.18-8.16 (m, 2H), 8.04 (s, 1H), 8.02-8.00 (m, 1H), 7.63-7.54 (m, 3H), 7.36-7.27 (m, 5H), 6.64 (d, 1H, J=9.1 Hz), 4.66 (s, 2H), 3.84 (m, 2H), 3.70 (m, 2H), 3.40 (m, 2H), 2.85 (m, 2H), 1.65 (s, 2H). MS (M+1): 536. 30 (m, 1H), 2.20 (m, 1H), 1.92 (m, 1H). MS (M+1): 555.1

### Example 113

### N-(6-(3-(3-(2-fluorophenyl)ureido)azetidin-1-yl)pyridin-3-yl)-2-phenyl-4-(trifluoromethyl)oxazole-5-carboxamide (159)

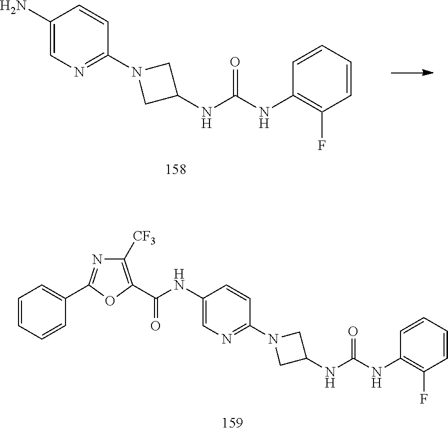

Intermediate 158 was prepared by the general procedures using 3-N—BOC-amino-azetidine. MS (M+1): 302.

Compound 159 was prepared by the general procedure for compound 62 by using HATU and 1-(1-(5-aminopyridin-2-yl)azetidin-3-yl)-3-(2-fluorophenyl)urea (158). ¹H NMR (500 MHz, DMSO-d6) δ 10.70 (s, 1H), 8.40 (s, 1H), 8.33 (s, 1H), 8.27 (d, 2H, J=8 Hz), 8.08 (t, 1H, J=8.5 Hz), 7.90 (d, 1H, J=9.5 Hz), 7.65 (m, 3H), 7.30 (d, 1H, J=6.5 Hz), 7.20 (t, 1H, J=10 Hz), 7.10 (t, 1H, J=8 Hz), 6.97 (q, 1H, J=7 Hz), 6.53 (d, 1H, J=8.5 Hz), 4.60 (m, 1H), 4.27 (t, 2H, J=8.5 Hz), 3.77 (t, 2H, J=8 Hz). MS (M+1): 541.2

### Example 114

### N-(6-(1-(2-fluorophenylcarbamoyl)azetidin-3-ylamino)pyridin-3-yl)-2-phenyl-4-(trifluoromethyl)oxazole-5-carboxamide (161)

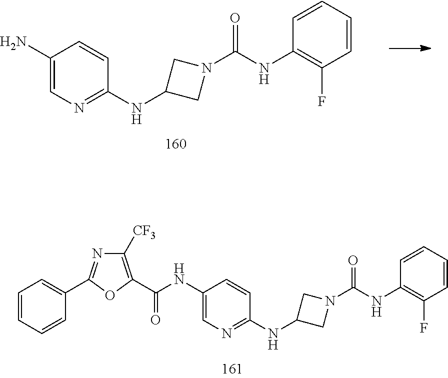

Intermediate 160 was prepared by the general procedures using 1-N-BOC-3-amino-azetidine. MS (M+1): 302.

Compound 161 was prepared by the general procedure for compound 62 by using HATU and 3-(5-aminopyridin-2-ylamino)-N-(2-fluorophenyl)azetidine-1-carboxamide (160). ¹H NMR (500 MHz, DMSO-d6) δ 10.63 (s, 1H), 8.30 (s, 1H), 8.27 (d, 2H, J=8 Hz) 8.20 (s, 1H), 7.77 (d, 1H, J=9 Hz), 7.67 (m, 3H), 7.60 (t, 1H, J=7.5 Hz), 7.32 (d, 1H, J=6 Hz), 7.20 (t, 1H, J=9.5 Hz), 7.10 (m, 2H), 6.58 (d, 1H, J=9 Hz), 4.55 (m, 1H), 4.30 (t, 2H, J=8 Hz), 3.83 (d, 1H, J=5.5 Hz), 3.81 (d, 1H, J=5.5 Hz). MS (M+1): 541.2

### Example 115

### ethyl 4,4,4-trifluoro-3-hydroxy-3-(4-(5-(2-phenyl-4-(trifluoromethyl)oxazole-5-carboxamido)pyridin-2-yl)piperazine-1-carbonyl)butanoate (162)

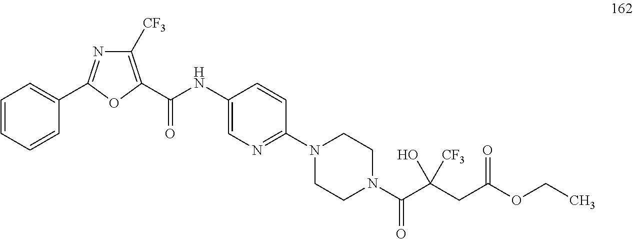

Compound 162 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.70 (d, 1H, J=2.5 Hz), 8.29 (m, 2H), 8.23 (m, 1H), 7.65 (m, 3H), 7.38 (d, 1H, J=9.8 Hz), 4.33 (m, 2H), 3.80 (m, 8H), 3.28 (m, 1H), 3.18 (m, 1H), 1.33 (t, 3H, J=7.3 Hz). MS (M+1): 630.3

### Example 116

### N-(6-(4-(2-hydroxy-3-(piperidin-1-yl)propanoyl)piperazin-1-yl)pyridin-3-yl)-2-phenyl-4-(trifluoromethyl)oxazole-5-carboxamide (163)

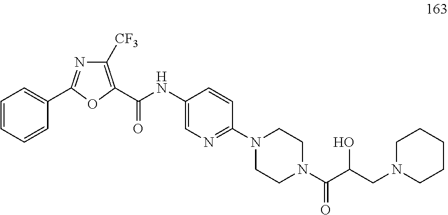

Compound 163 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.62 (d, 1H, J=2.2 Hz), 8.29 (m, 2H), 8.15 (m, 1H), 7.65 (m, 3H), 7.22 (d, 1H, J=9.5 Hz), 4.96 (m, 1H), 3.80 (m, 10H), 3.40 (d, 2H, J=6.0 Hz), 3.10 (m, 2H), 1.91 (m, 6H). MS (M+1): 573.3

### Example 117

### 2-phenyl-N-(6-(4-(3,3,3-trifluoro-2-hydroxypropanoyl)piperazin-1-yl)pyridin-3-yl)-4-(trifluoromethyl)oxazole-5-carboxamide (164)

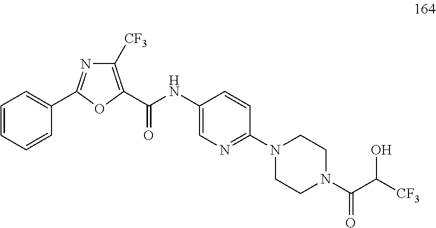

Compound 164 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.68 (d, 1H, J=2.5 Hz), 8.29 (m, 2H), 8.21 (m, 1H), 7.65 (m, 3H), 7.38 (d, 1H, J=9.8 Hz), 5.12 (q, 1H, J=6.9 Hz), 3.94 (m, 3H), 3.78 (m, 5H). MS (M+1): 544.3

### Example 118

### (R)—N-(6-(4-(2-hydroxy-2-phenylacetyl)piperazin-1-yl)pyridin-3-yl)-2-phenyl-4-(trifluoromethyl)oxazole-5-carboxamide (165)

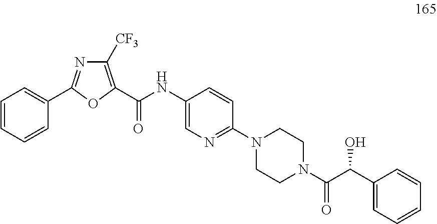

Compound 165 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.63 (d, 1H, J=2.2 Hz), 8.27 (m, 2H), 8.17 (m, 1H), 7.65 (m, 3H), 7.45 (m, 4H), 7.37 (m, 1H), 7.27 (d, 1H, J=9.5 Hz), 5.54 (s, 1H), 3.81 (m, 4H), 3.58 (m, 3H), 3.25 (m, 1H), MS (M+1): 552.3

### Example 119

### 2-hydroxy-3-oxo-3-(4-(5-(2-phenyl-4-(trifluoromethyl)oxazole-5-carboxamido)pyridin-2-yl)piperazin-1-yl)propanoic acid (166)

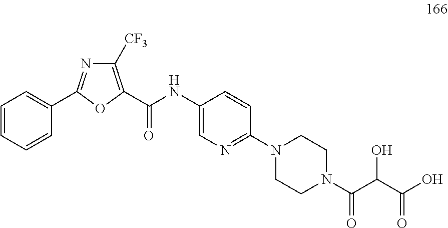

Compound 166 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.66 (d, 1H, J=2.5 Hz), 8.29 (m, 2H), 8.19 (m, 1H), 7.65 (m, 3H), 7.31 (d, 1H, J=9.8 Hz), 5.14 (s, 1H), 3.85 (m, 8H). MS (M+1): 520.3

### Example 120

### N-(6-(4-(2-(3,4-difluorophenyl)-2-hydroxyacetyl)piperazin-1-yl)pyridin-3-yl)-2-phenyl-4-(trifluoromethyl)oxazole-5-carboxamide (167)

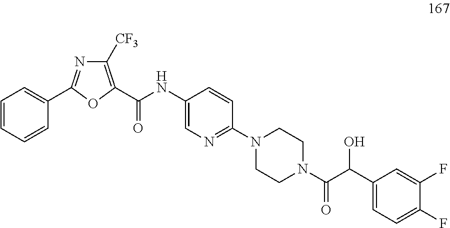

Compound 167 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.66 (d, 1H, J=2.2 Hz), 8.27 (m, 2H), 8.17 (m, 1H), 7.65 (m, 3H), 7.41 (m, 1H), 7.31 (m, 3H), 5.54 (s, 1H), 3.83 (m, 3H), 3.71 (m, 3H), 3.54 (m, 2H). MS (M+1): 588.3

### Example 121

### N-(6-(4-(2-(4-chlorophenyl)-2-hydroxyacetyl)piperazin-1-yl)pyridin-3-yl)-2-phenyl-4-(trifluoromethyl)oxazole-5-carboxamide (168)

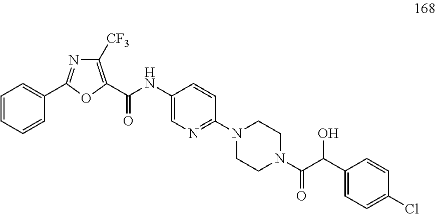

Compound 168 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.65 (d, 1H, J=2.2 Hz), 8.27 (m, 2H), 8.17 (m, 1H), 7.65 (m, 3H), 7.45 (m, 4H), 7.29 (d, 1H, J=9.8 Hz), 5.54 (s, 1H), 3.70 (m, 7H), 3.37 (m, 1H). MS (M+1): 586.3

### Example 122

### (R)—N-(6-(4-(2-cyclohexyl-2-hydroxyacetyl)piperazin-1-yl)pyridin-3-yl)-2-phenyl-4-(trifluoromethyl)oxazole-5-carboxamide (169)

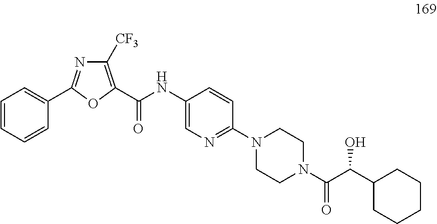

Compound 169 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.57 (d, 1H, J=2.5 Hz), 8.27 (m, 2H), 8.09 (m, 1H), 7.65 (m, 3H), 7.12 (d, 1H, J=9.1 Hz), 4.27 (d, 1H, J=5.6 Hz), 3.85 (m, 3H), 3.70 (m, 5H), 1.80 (m, 3H), 1.68 (m, 3H), 1.24 (m, 5H). MS (M+1): 558.3

### Example 123

### (S)—N-(6-(4-(2-cyclohexyl-2-hydroxyacetyl)piperazin-1-yl)pyridin-3-yl)-2-phenyl-4-(trifluoromethyl)oxazole-5-carboxamide (170)

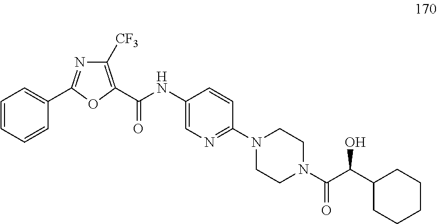

Compound 170 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.57 (d, 1H, J=2.5 Hz), 8.27 (m, 2H), 8.09 (m, 1H), 7.65 (m, 3H), 7.11 (d, 1H, J=9.5 Hz), 4.27 (d, 1H, J=5.6 Hz), 3.85 (m, 3H), 3.70 (m, 5H), 1.80 (m, 3H), 1.68 (m, 3H), 1.24 (m, 5H). MS (M+1): 558.3

### Example 124

### N-(6-(4-(2-hydroxy-3-phenylpropanoyl)piperazin-1-yl)pyridin-3-yl)-2-phenyl-4-(trifluoromethyl)oxazole-5-carboxamide (171)

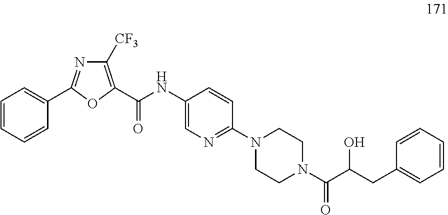

Compound 171 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.56 (d, 1H, J=2.5 Hz), 8.28 (m, 2H), 8.09 (m, 1H), 7.65 (m, 3H), 7.31 (m, 4H), 7.23 (m, 1H), 7.08 (d, 1H, J=9.5 Hz), 4.77 (t, 1H, J=6.9 Hz), 3.69 (m, 4H), 3.51 (m, 3H), 3.26 (m, 1H), 3.02 (m, 2H). MS (M+1): 566.3

### Example 125

### 4,4,4-trifluoro-3-hydroxy-3-(4-(5-(2-phenyl-4-(trifluoromethyl)oxazole-5-carboxamido)pyridin-2-yl)piperazine-1-carbonyl)butanoic acid (172)

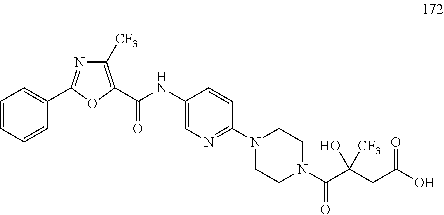

Compound 172 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.58 (d, 1H, J=2.5 Hz), 8.29 (m, 2H), 8.11 (m, 1H), 7.65 (m, 3H), 7.15 (d, 1H, J=9.8 Hz), 3.72 (m, 8H), 3.28 (d, 1H, J=16 Hz), 1.15 (d, 1H, J=16 Hz). MS (M+1): 602.3

### Example 126

### N-(6-(4-(2-hydroxy-2-(4-(trifluoromethyl)phenyl)acetyl)piperazin-1-yl)pyridin-3-yl)-2-phenyl-4-(trifluoromethyl)oxazole-5-carboxamide (173)

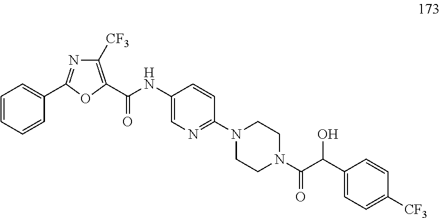

Compound 173 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.62 (d, 1H, J=2.5 Hz), 8.27 (m, 2H), 8.15 (m, 1H), 7.67 (m, 7H), 7.23 (d, 1H, J=9.8 Hz), 5.65 (s, 1H), 3.74 (m, 6H), 3.49 (m, 2H). MS (M+1): 620.3

### Example 127

### N-(6-(4-(2-(3,5-difluorophenyl)-2-hydroxyacetyl)piperazin-1-yl)pyridin-3-yl)-2-phenyl-4-(trifluoromethyl)oxazole-5-carboxamide (174)

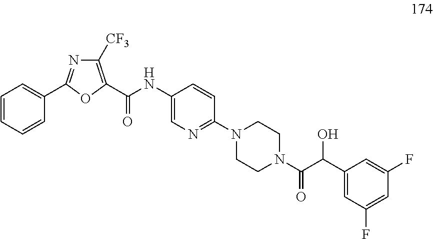

Compound 174 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.62 (d, 1H, J=2.5 Hz), 8.27 (m, 2H), 8.15 (m, 1H), 7.64 (m, 3H), 7.23 (d, 1H, J=9.8 Hz), 7.11 (m, 2H), 6.96 (m, 1H), 5.57 (s, 1H), 3.74 (m, 6H), 3.53 (m, 2H). MS (M+1): 588.3

### Example 128

### N-(6-(4-(2-hydroxy-2-(4-fluorophenyl)acetyl)piperazin-1-yl)pyridin-3-yl)-2-phenyl-4-(trifluoromethyl)oxazole-5-carboxamide (175)

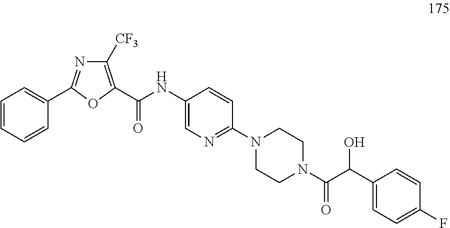

Compound 175 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.68 (d, 1H, J=2.2 Hz), 8.27 (m, 2H), 8.15 (m, 1H), 7.64 (m, 3H), 7.50 (m, 2H), 7.29 (d, 1H, J=9.8 Hz), 7.16 (m, 2H), 5.55 (s, 1H), 3.71 (m, 8H). MS (M+1): 570.3

### Example 129

### N-(6-(4-(2-(benzo[d][1,3]dioxol-5-yl)-2-hydroxyacetyl)piperazin-1-yl)pyridin-3-yl)-2-phenyl-4-(trifluoromethyl)oxazole-5-carboxamide (176)

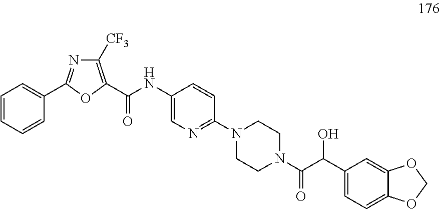

Compound 176 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.64 (d, 1H, J=2.2 Hz), 8.27 (m, 2H), 8.18 (m, 1H), 7.64 (m, 3H), 7.28 (d, 1H, J=9.8 Hz), 6.95 (m, 2H), 6.86 (d, 1H, J=8.2 Hz), 5.97 (s, 2H), 5.45 (s, 1H), 3.64 (m, 8H). MS (M+1): 596.3

### Example 130

### N-(6-(4-(2-(2,5-dimethylphenyl)-2-hydroxyacetyl)piperazin-1-yl)pyridin-3-yl)-2-phenyl-4-(trifluoromethyl)oxazole-5-carboxamide (177)

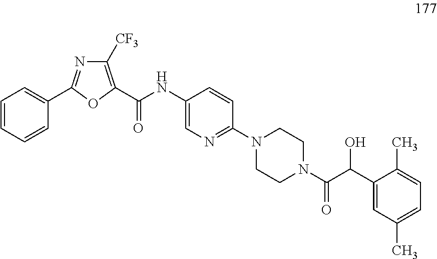

Compound 177 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.63 (d, 1H, J=2.2 Hz), 8.27 (m, 2H), 8.17 (m, 1H), 7.64 (m, 3H), 7.25 (d, 1H, J=9.8 Hz), 7.16 (d, 1H, J=7.6 Hz), 7.09 (d, 1H, J=7.8 Hz), 7.06 (s, 1H), 5.60 (s, 1H), 3.99 (m, 1H), 3.81 (m, 2H), 3.60 (m, 3H), 3.33 (m, 1H) 3.10 (m, 1H), 2.45 (s, 3H), 2.30 (s, 3H). MS (M+1): 580.3

### Example 131

### 2,3-dihydroxy-4-oxo-4-(4-(5-(2-phenyl-4-(trifluoromethyl)oxazole-5-carboxamido)pyridin-2-yl)piperazin-1-yl)butanoic acid (178)

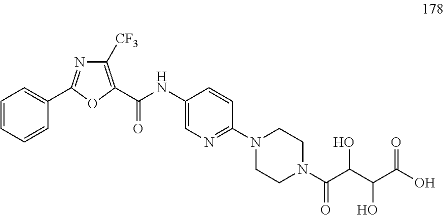

Compound 178 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.55 (d, 1H, J=2.5 Hz), 8.29 (m, 2H), 8.06 (m, 1H), 7.65 (m, 3H), 7.07 (d, 1H, J=9.1 Hz), 4.90 (d, 1H, J=2.8 Hz), 4.46 (d, 1H, J=2.8 Hz), 3.82 (m, 8H). MS (M+1): 550.3

### Example 132

### (R)—N-(6-(4-(2-hydroxy-2-phenylacetyl)-1,4-diazepan-1-yl)pyridin-3-yl)-2-phenyl-4-(trifluoromethyl)oxazole-5-carboxamide (179)

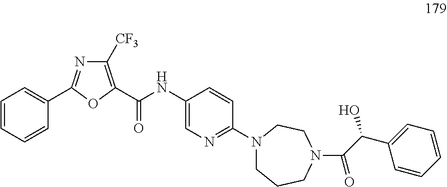

Compound 179 was prepared by using N-(6-(1,4-diazepan-1-yl)pyridin-3-yl)-2-phenyl-4-(trifluoromethyl)oxazole-5-carboxamide. ¹H NMR (500 MHz, CD₃OD) δ 8.37 (d, 1H, J=2.5 Hz), 8.29 (m, 2H), 7.88 (m, 1H), 7.64 (m, 3H), 7.37 (m, 3H), 7.25 (m, 2H), 6.75 (d, 1H, J=9.1 Hz), 5.37 (s, 1H), 4.04 (m, 1H), 3.60 (m, 7H), 1.95 (m, 1H), 1.51 (m, 1H). MS (M+1): 566.3

### Example 133

### (R)—N-(6-(4-(2-cyclohexyl-2-hydroxyacetyl)-1,4-diazepan-1-yl)pyridin-3-yl)-2-phenyl-4-(trifluoromethyl)oxazole-5-carboxamide (180)

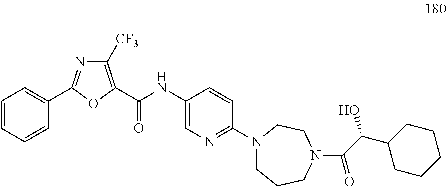

Compound 180 was prepared by using N-(6-(1,4-diazepan-1-yl)pyridin-3-yl)-2-phenyl-4-(trifluoromethyl)oxazole-5-carboxamide. ¹H NMR (500 MHz, CD₃OD) δ 8.52 (d, 1H, J=2.5 Hz), 8.29 (m, 2H), 8.04 (m, 1H), 7.64 (m, 3H), 6.94 (d, 1H, J=9.4 Hz), 4.10 (d, 1H, J=6.9 Hz), 3.78 (m, 8H), 1.74 (m, 7H), 1.20 (m, 6H). MS (M+1): 572.3

### Example 134

### 2-phenyl-N-(6-(4-(3,3,3-trifluoro-2-hydroxypropanoyl)-1,4-diazepan-1-yl)pyridin-3-yl)-4-(trifluoromethyl)oxazole-5-carboxamide (181)

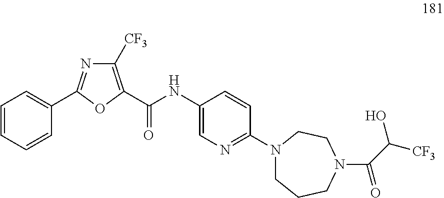

Compound 181 was prepared by using N-(6-(1,4-diazepan-1-yl)pyridin-3-yl)-2-phenyl-4-(trifluoromethyl)oxazole-5-carboxamide. ¹H NMR (500 MHz, CD₃OD) δ 8.46 (d, 1H, J=2.5 Hz), 8.29 (m, 2H), 7.95 (m, 1H), 7.64 (m, 3H), 6.88 (d, 1H, J=9.8 Hz), 4.92 (m, 1H), 3.82 (m, 6H), 3.35 (m, 2H), 2.04 (m, 1H), 1.34 (m, 1H). MS (M+1): 558.3

### Example 135

### (R)—N-(6-(4-(2-(3,4-difluorophenyl)-2-hydroxyacetyl)-1,4-diazepan-1-yl)pyridin-3-yl)-2-phenyl-4-(trifluoromethyl)oxazole-5-carboxamide (182)

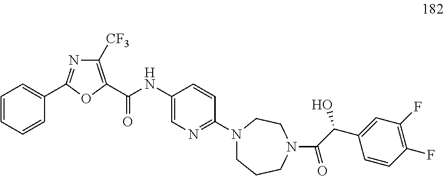

Compound 182 was prepared by using N-(6-(1,4-diazepan-1-yl)pyridin-3-yl)-2-phenyl-4-(trifluoromethyl)oxazole-5-carboxamide. ¹H NMR (500 MHz, CD₃OD) δ 8.39 (s, 1H), 8.29 (m, 2H), 7.90 (m, 1H), 7.64 (m, 3H), 7.19 (m, 3H), 6.75 (d, 1H, J=9.1 Hz), 5.35 (s, 1H), 3.99 (m, 1H), 3.67 (m, 7H), 1.90 (m, 1H), 1.63 (m, 1H). MS (M+1): 602.3

### Example 136

### N-(6-(4-(2-hydroxy-2-(3-hydroxyphenyl)acetyl)piperazin-1-yl)pyridin-3-yl)-2-phenyl-4-(trifluoromethyl)oxazole-5-carboxamide (183)

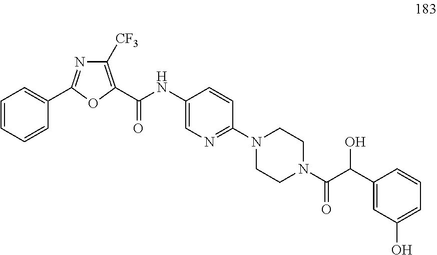

Compound 183 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.45 (d, 1H, J=2.5 Hz), 8.27 (m, 2H), 7.97 (m, 1H), 7.64 (m, 3H), 7.23 (t, 1H, J=7.9 Hz), 6.90 (m, 3H), 6.78 (m, 1H), 5.43 (s, 1H), 3.85 (m, 1H), 3.61 (m, 4H), 3.42 (m, 2H), 3.06 (m, 1H). MS (M+1): 568.3

### Example 137

### N-(6-(4-(2-(2-fluorophenyl)-2-hydroxyacetyl)piperazin-1-yl)pyridin-3-yl)-2-phenyl-4-(trifluoromethyl)oxazole-5-carboxamide (184)

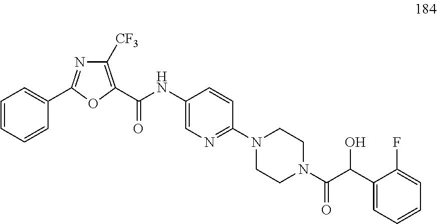

Compound 184 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.56 (d, 1H, J=2.2 Hz), 8.28 (m, 2H), 8.09 (m, 1H), 7.64 (m, 3H), 7.48 (t, 1H, J=7.6 Hz), 7.42 (m, 1H), 7.25 (t, 1H, J=7.6 Hz), 7.19 (t, 1H, J=9.5 Hz), 7.12 (d, 1H, J=9.5 Hz), 5.80 (s, 1H), 3.70 (m, 8H). MS (M+1): 570.3

### Example 138

### N-(6-(4-(2-(2,5-difluorophenyl)-2-hydroxyacetyl)piperazin-1-yl)pyridin-3-yl)-2-phenyl-4-(trifluoromethyl)oxazole-5-carboxamide (185)

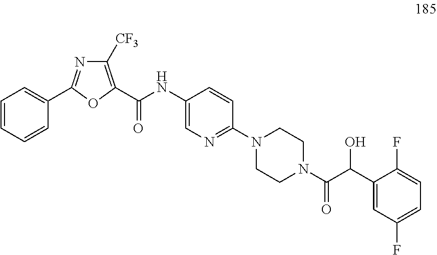

Compound 185 was prepared by using 2-phenyl-N-(6-(piperazin-1-yl)pyridin-3-0)-4-(trifluoromethyl)oxazole-5-carboxamide. ¹H NMR (500 MHz, CD₃OD) δ 8.56 (d, 1H, J=2.2 Hz), 8.28 (m, 2H), 8.09 (m, 1H), 7.64 (m, 3H), 7.19 (m, 4H), 5.80 (s, 1H), 3.80 (m, 3H), 3.63 (m, 4H), 3.41 (m, 1H). MS (M+1): 588.3

### Example 139

### 2-phenyl-N-(6-(4-(3,3,3-trifluoro-2-hydroxy-2-methylpropanoyl)piperazin-1-yl)pyridin-3-yl)-4-(trifluoromethyl)oxazole-5-carboxamide (186)

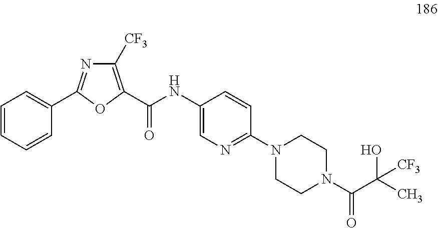

Compound 186 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.66 (d, 1H, J=2.5 Hz), 8.29 (m, 2H), 8.19 (m, 1H), 7.65 (m, 3H), 7.31 (d, 1H, J=9.5 Hz), 4.33 (m, 2H), 3.78 (m, 6H), 1.67 (s, 3H). MS (M+1): 558.3

### Example 140

### N-(6-(4-(2-hydroxy-2-phenylpropanoyl)piperazin-1-yl)pyridin-3-yl)-2-phenyl-4-(trifluoromethyl)oxazole-5-carboxamide (187)

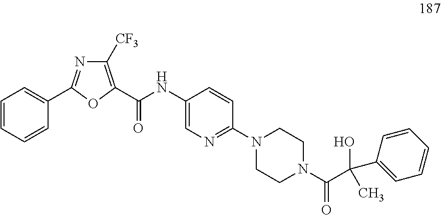

Compound 187 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.55 (d, 1H, J=2.5 Hz), 8.27 (m, 2H), 8.08 (m, 1H), 7.64 (m, 3H), 7.42 (m, 4H), 7.30 (t, 1H, J=7.6 Hz), 7.11 (d, 1H, J=9.5 Hz), 3.69 (m, 6H), 3.37 (m, 1H), 2.90 (m, 1H), 1.67 (s, 3H). MS (M+1): 566.3

### Example 141

### N-(6-(4-(2-hydroxy-2-(4-hydroxyphenyl)acetyl)piperazin-1-yl)pyridin-3-yl)-2-phenyl-4-(trifluoromethyl)oxazole-5-carboxamide (188)

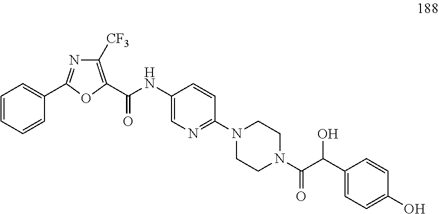

Compound 188 was prepared by using 2-phenyl-N-(6-(piperazin-1-yl)pyridin-3-yl)-4-(trifluoromethyl)oxazole-5-carboxamide. ¹H NMR (500 MHz, CD₃OD) δ 8.58 (d, 1H, J=2.5 Hz), 8.29 (m, 2H), 8.11 (m, 1H), 7.64 (m, 3H), 7.28 (d, 2H, J=8.8 Hz), 7.15 (d, 1H, J=9.5 Hz), 6.82 (d, 2H, J=8.5 Hz), 5.43 (s, 1H), 3.73 (m, 7H), 3.13 (m, 1H). MS (M+1): 568.3

### Example 142

### N-[6-[4-(2(S)-hydroxy-3-methyl-1-oxobutyl)-1-piperazinyl]-3-pyridinyl]-2-phenyl-4-(trifluoromethyl)-5-oxazolecarboxamide (189)

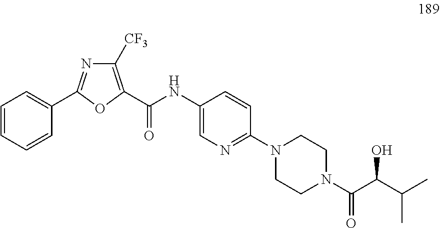

2-Phenyl-N-(6-(piperazin-1-yl)pyridin-3-yl)-4-(trifluoromethyl)oxazole-5-carboxamide HCl salt (46 mg, 0.1 mmol) was mixed with L-α-hydroxyisovaleric acid (12 mg, 0.1 mmol), diisopropylethylamine (0.05 mL), and HATU (57 mg, 0.15 mmol) in 1 mL of dry DMF. The mixture was stirred at room temperature for 3 h, diluted with 2 mL of DMF, and then subjected to Gilson HPLC purification to give 38 mg of pure product 189.

¹H NMR (500 MHz, CDCl₃) δ 8.32 (d, 1H, J=2.8 Hz), 8.19 (d, 2H), 8.10 (dd, 1H), 7.98 (s, 1H), 7.63 (m, 1H), 7.58 (m, 2H), 6.74 (d, 11-1H), 4.33 (s, 1H), 3.83 (m, 2H), 3.61 (m, 6H), 1.90 (m, 1H), 1.13 (d, 3H, J=6.9 Hz), 0.85 (d, 3H, J=6.9 Hz). MS (M+1): 518.3

### Example 143

### N-[6-[4-(2(R)-hydroxy-3,3-dimethyl-1-oxobutyl)-1-piperazinyl]-3-pyridinyl]-2-phenyl-4-(trifluoromethyl)-5-oxazolecarboxamide (190)

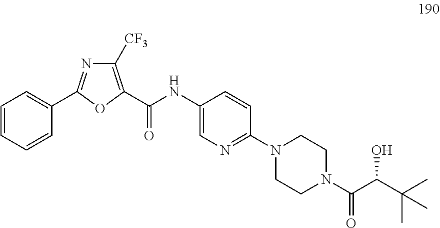

Compound 190 was prepared by the general procedure for compound 189.

¹H NMR (500 MHz, CDCl₃) δ 8.32 (d, 1H, J=2.5 Hz), 8.18 (d, 2H), 8.08 (m, 2H), 7.58 (m, 3H), 6.74 (m, 1H), 4.26 (s, 1H), 3.99 (m, 1H), 3.69 (m, 5H), 3.54 (m, 2H), 1.02 (s, 9H). MS (M+1): 532.3

### Example 144

### N-[6-[4-(2-hydroxyacetyl)-1-piperazinyl]-3-pyridinyl]-2-phenyl-4-(trifluoromethyl)-5-oxazolecarboxamide (191)

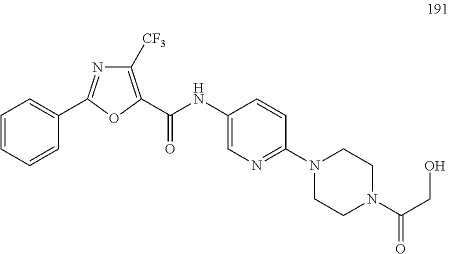

Compound 191 was prepared by the general procedure for compound 189.

MS: 476.3 (M+1)

### Example 145

### N-[4-[6-[hydroxy(phenyl)methyl]-3-pyridinyl]phenyl]-2-phenyl-4-(trifluoromethyl)-5-oxazolecarboxamide (195)

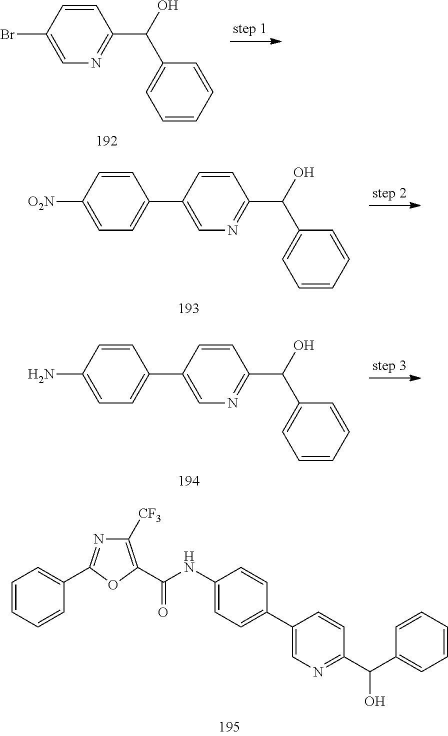

Step 1: (5-(4-nitrophenyl)pyridin-2-yl)(phenyl)methanol (193)

Compound 192 (0.26 g, 1 mmol), p-nitrophenylboronic acid (170 mg, 1 mmol), Pd(PPh₃)₂Cl₂ (50 mg), K₂CO₃ (280 mg, 2 mmol) were mixed in a microwave reaction vial. The vial was capped, and the air was removed by vacuum through a needle, and back-filled with nitrogen (3 times). CH₃CN (8 mL) and water (2 mL) was introduced via syringe. The mixture was heated to 90° C. for 12 h then diluted with ethyl acetate, washed with brine, dried over sodium sulfate, and filtered. The solution was concentrated to give a product, which was used directly in the next step. LCMS: 307 (M+1)

Step 2: (5-(4-aminophenyl)pyridin-2-yl)(phenyl)methanal (194) PtO₂ (30 mg) was added to compound 193 (˜1 mmol) in a mixed solvent of 3:1 EtOAc:MeOH (24 mL). The resulting mixture was stirred under a balloon of hydrogen at r.t. overnight. LCMS shows all starting material was converted into aminopyridine derivative. The mixture was filtered, and the filtrate was concentrated to give a crude product that was purified by flash chromatography to give compound 194. LCMS: 277.2 (M+1)

Step 3: N-(4-(6-(hydroxy(phenyl)methyl)pyridin-3-yl)phenyl)-2-phenyl-4-(trifluoromethyl)oxazole-5-carboxamide (195)

Compound 194 (41 mg, 0.15 mmol) was mixed with the oxazole acid (39 mg, 0.15 mmol), diisopropylethylamine (0.06 mL), and HATU (76 mg, 0.2 mmol) in dry DMF (3 mL). The mixture was stirred at room temperature for 2 h and then subjected to Gilson HPLC purification to give 55 mg of the product 195. ¹H NMR (500 MHz, DMSO-d6) δ 10.91 (s, 1H), 8.87 (s, 1H), 8.28 (m, 3H), 7.92 (d, 2H, J=8.5 Hz), 7.83 (d, 2H, J=8.5 Hz), 7.76 (d, 1H, J=8.2 Hz), 7.68 (m, 3H), 7.47 (d, 2H, J=7.9 Hz), 7.35 (t, 2H, J=7.5 Hz), 7.26 (t, 1H, J=7.6 Hz), 5.88 (s, 1H). MS (M+1): 516.3

### Example 146

### N-[6-[4-(2,1-benzisoxazol-3-ylcarbonyl)-1-piperazinyl]-3-pyridinyl]-2-phenyl-4-(trifluoromethyl)-5-oxazolecarboxamide (196)

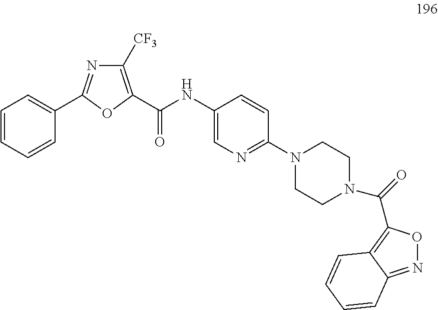

Compound 196 was prepared by the general procedure for compound 189.

¹H NMR (500 MHz, DMSO-d6) δ 10.73 (s, 1H), 8.47 (d, 1H, J=2.5 Hz), 8.27 (m, 2H), 7.96 (m, 1H), 7.87 (m, 1H), 7.79 (m, 1H), 7.67 (m, 3H), 7.52 (m, 1H), 7.28 (m, 1H), 6.99 (d, 1H, J=9.1 Hz), 3.89 (m, 4H), 3.70 (m, 4H). MS (M+1): 563.3

### Example 147

### Ethyl 5,6,7,8-tetrahydro-7-[5-[[[2-phenyl-4-(trifluoromethyl)-5-oxazolyl]carbonyl]amino]-2-pyridinyl]-1,2,4-triazolo[4,3-a]pyrazine-3-carboxylate (199)

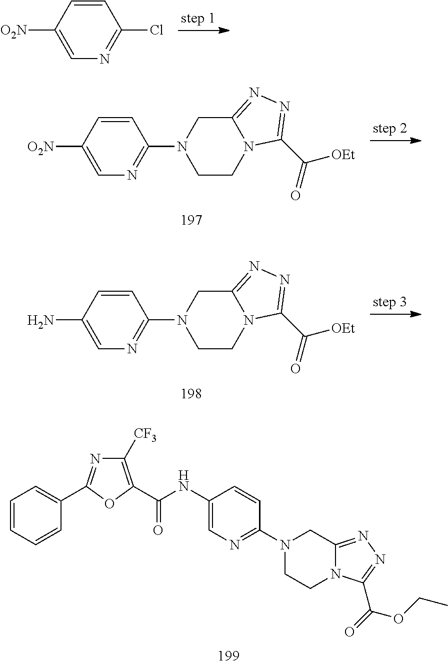

Step 1: ethyl 7-(5-nitropyridin-2-yl)-5,6,7,8-tetrahydro-[1,2,4]triazolo[4,3-a]pyrazine-3-carboxylate (197)

2-Chloro-5-nitropyridine (0.97 g, 5.5 mmol), ethyl 5,6,7,8-tetrahydro-[1,2,4]triazolo[4,3-a]pyrazine-3-carboxylate (1.0 g, 5.1 mmol), and diisopropylethylamine (2 mL) were mixed in acetonitrile (5 mL), and heated to 80° C. for one h. The mixture was poured into water, and the precipitate was collected by filtration. The precipitate was washed with water, then by ether, and dried in a vacuum oven overnight to give 1.2 gram of product 197 (75% yield).

Step 2: ethyl 7-(5-aminopyridin-2-yl)-5,6,7,8-tetrahydro-[1,2,4]triazolo[4,3-a]pyrazine-3-carboxylate (198)

Compound 197 (1.1 g) was reduced by stirring with PtO₂ (20 mg) in 3:1 EtOAc:MeOH (40 mL) under a balloon of hydrogen at r.t. overnight. The solid was filtered off, and the filtrate was concentrated to give 1.0 gram of the aminopyridine product 198 which was used in the next step without further purification.

Step 3: ethyl 5,6,7,8-tetrahydro-7-[5-[[[2-phenyl-4-(trifluoromethyl)-5-oxazolyl]carbonyl]amino]-2-pyridinyl]-1,2,4-triazolo[4,3-a]pyrazine-3-carboxylate

(199)

Compound 198 (37 mg, 0.13 mmol) was mixed with oxazole acid (33 mg, 0.13 mmol), diisopropylethylamine (0.07 mL), and HATU (64 mg, 0.17 mmol) in dry DMF (2 mL). The mixture was stirred at room temperature overnight then diluted with 2 mL of DMF, and subjected to Gilson HPLC purification to give 55 mg of the product 199 as the TFA salt. ¹H NMR (500 MHz, DMSO-d6) δ 10.77 (s, 1H), 8.51 (m, 1H), 8.27 (m, 2H), 8.00 (m, 1H), 7.67 (m, 3H), 7.19 (m, 1H), 4.98 (s, 2H), 4.38 (m, 4H), 4.07 (m, 2H), 1.34 (t, 3H, J=7.1 Hz). MS (M+1): 528.3

### Example 148

### N-cyclopentyl-5,6,7,8-tetrahydro-7-[5-[[[2-phenyl-4-(trifluoromethyl)-5-oxazolyl]carbonyl]amino]-2-pyridinyl]-1,2,4-triazolo[4,3-a]pyrazine-3-carboxamide (201)

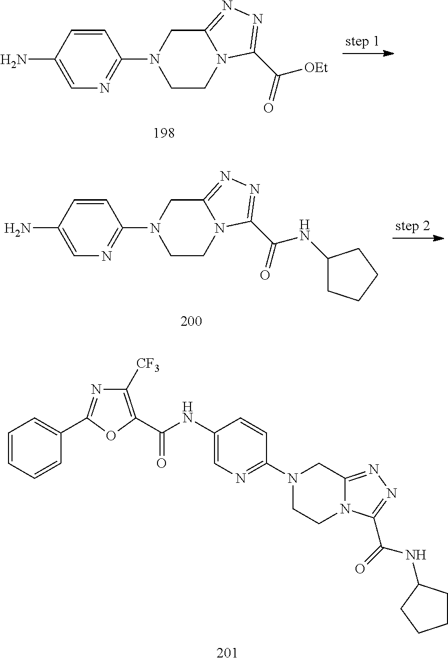

Step 1: 7-(5-aminopyridin-2-yl)-N-cyclopentyl-5,6,7,8-tetrahydro-[1,2,4]triazolo[4,3-a]pyrazine-3-carboxamide (200)

Compound 198 (58 mg, 0.2 mmol) was mixed with 0.2 mL of cyclopentylamine in a capped microwave reaction vial. The mixture was heated to 170° C. overnight then cooled and diluted with ether/hexane. The precipitate was collected by filtration, and dried in a vacuum oven at 50° C. overnight, to give 52 mg of the product 200.

Step 2: N-cyclopentyl-5,6,7,8-tetrahydro-7-[5-[[[2-phenyl-4-(trifluoromethyl)-5-oxazolyl]carbonyl]amino]-2-pyridinyl]-1,2,4-triazolo[4,3-a]pyrazine-3-carboxamide (201)

Compound 201 was prepared by the general procedure for compound 199.

¹H NMR (500 MHz, DMSO-d6) δ 10.77 (s, 1H), 8.51 (m, 1H), 8.27 (m, 2H), 8.00 (m, 1H), 7.67 (m, 3H), 7.19 (m, 1H), 4.98 (s, 2H), 4.38 (m, 4H), 4.07 (m, 2H), 1.34 (t, 3H, J=7.1 Hz). MS (M+1): 528.3

### Example 149

### N-[6-[3-(3-fluorophenyl)-5,6-dihydro-1,2,4-triazolo[4,3-a]pyrazin-7(8H)-yl]-3-pyridinyl]-2-phenyl-4-(trifluoromethyl)-5-oxazolecarboxamide (206)

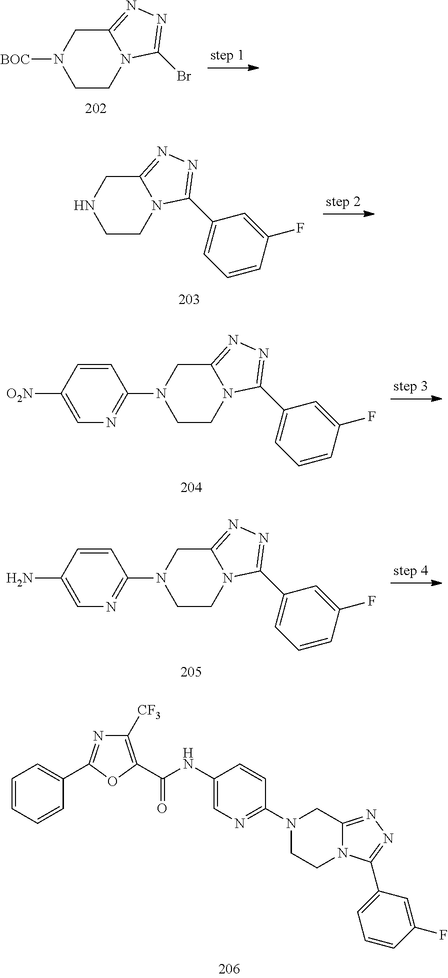

Step 1: 3-(3-fluorophenyl)-5,6,7,8-tetrahydro-[1,2,4]triazolo[4,3-a]pyrazine

(203)

Tert-butyl 3-bromo-5,6-dihydro-[1,2,4]triazolo[4,3-a]pyrazine-7(8H)-carboxylate (202) (0.12 g, 0.4 mmol), m-fluorophenylboronic acid (70 mg, 0.5 mmol), Pd(PPh₃)₂Cl₂ (20 mg), K₂CO₃ (110 mg, 0.8 mmol) were mixed in a microwave reaction vial. The vial was capped and air was removed by vacuum through a needle, and back-filled with nitrogen (3 times). CH₃CN (3 mL) and water (0.6 mL) was introduced via syringe. The mixture was then heated to 90° C. for 10 h then diluted with ethyl acetate, washed with brine, dried over sodium sulfate, and concentrated. The crude product was purified by flash chromatography to give compound 203 which was treated with 4 N HCl in dioxane at r.t. for 2 h to give the HCl salt.

Step 2: 3-(3-fluorophenyl)-7-(5-nitropyridin-2-yl)-5,6,7,8-tetrahydro-[1,2,4]triazolo[4,3-a]pyrazine (204)

Compound 203 HCl salt (˜0.3 mmol) in EtOH (6 mL) was mixed with 2-chloro-5-nitropyridine (71 mg, 0.45 mmol) and diisopropylethylamine (0.17 mL, 1 mmol). The resulting mixture was heated to 80° C. for 10 h then cooled and concentrated. The solid was washed with water and ethanol/hexane to give a solid product which was used in the next step without further purification.

Step 3: 6-(3-(3-fluorophenyl)-5,6-dihydro-[1,2,4]triazolo[4,3-a]pyrazin-7(8H)-yl)pyridin-3-amine (205)

Compound 205 was prepared by the general procedure for compound 198.

Step 4: N-[6-[3-(3-fluorophenyl)-5,6-dihydro-1,2,4-triazolo[4,3-a]pyrazin-7(8H)-yl]-3-pyridinyl]-2-phenyl-4-(trifluoromethyl)-5-oxazolecarboxamide (206)

Compound 206 was prepared by the general procedure for compound 199.

¹H NMR (500 MHz, DMSO-d6) δ 10.78 (s, 1H), 8.54 (m, 1H), 8.27 (m, 2H), 8.02 (m, 1H), 7.66 (m, 5H), 7.41 (m, 1H), 7.19 (d, 1H), 5.00 (s, 2H), 4.31 (t, 2H, J=5.2 Hz), 4.08 (t, 2H, J=5.2 Hz). MS (M+1): 550.3

### Example 150

### N-[6-[5,6-dihydro-3-[hydroxy(phenyl)methyl]-1,2,4-triazolo[4,3-a]pyrazin-7(8H)-yl]-3-pyridinyl]-2-phenyl-4-(trifluoromethyl)-5-oxazolecarboxamide (210)

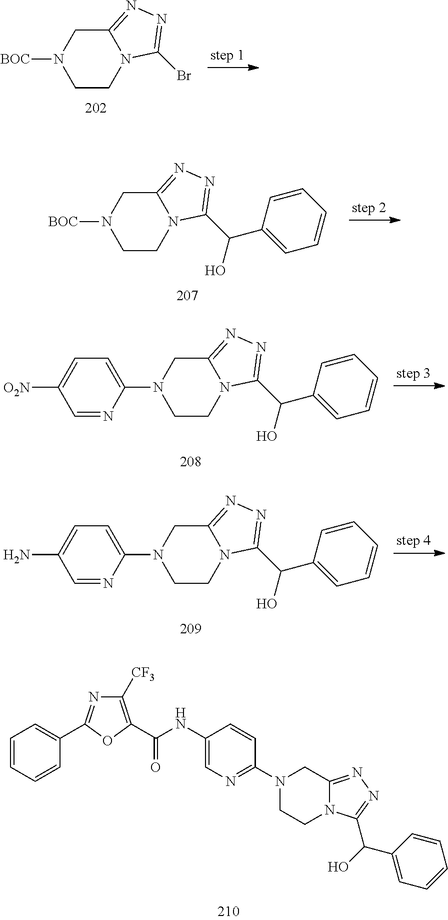

Step 1: phenyl(5,6,7,8-tetrahydro-[1,2,4]triazolo[4,3-a]pyrazin-3-yl)methanol (207)

Tert-butyl 3-bromo-5,6-dihydro-[1,2,4]triazolo[4,3-a]pyrazine-7(8H)-carboxylate (202) (0.12 g, 0.4 mmol) was placed in a flame-dried flask dry THF (5 mL) under nitrogen and cooled to −78° C. n-BuLi in hexane (2.5 M solution, 0.32 mL, 0.8 mmol) was added. The mixture was stirred at −78° C. for 40 mins then benzaldehyde (85 mg, 0.8 mmol) was added. The reaction was stirred at −78° C. for an additional 30 mins then quenched by the addition of saturated NH₄Cl solution at −78° C. and warmed up to room temperature. The product was extracted with EtOAc, washed with water and brine, dried (Na₂SO₄) and concentrated. The crude product was treated with 4 N HCl solution in dioxane at room temperature for 3 h then concentrated and dried in a vacuum oven at 50° C. for 3 h before use in the next step. LCMS: 231 (M+1)

Step 2: (7-(5-nitropyridin-2-yl)-5,6,7,8-tetrahydro-[1,2,4]triazolo[4,3-a]pyrazin-3-yl)(phenyl)methanol (208)

Compound 208 was prepared by the general procedure for compound 204.

LCMS: 353 (M+1)

Step 3: (7-(5-aminopyridin-2-yl)-5,6,7,8-tetrahydro-[1,2,4]triazolo[4,3-a]pyrazin-3-yl)(phenyl)methanol (209)

Compound 209 was prepared by the general procedure for compound 198.

LCMS: 323 (M+1)

Step 4: N-[6-[5,6-dihydro-3-[hydroxy(phenyl)methyl]-1,2,4-triazolo[4,3-a]pyrazin-7(8H)-yl]-3-pyridinyl]-2-phenyl-44 trifluoromethyl)-5-oxazolecarboxamide (210)

Compound 210 was prepared by the general procedure for compound 199.

¹H NMR (500 MHz, DMSO-d6) δ 10.74 (s, 1H), 8.48 (d, 1H, J=2.5 Hz), 826 (d, 2H, J=7.3 Hz), 7.96 (m, 1H), 7.66 (m, 3H), 7.38 (m, 4H), 7.30 (m, 1H), 7.12 (d, 1H, J=9.1 Hz), 6.49 (s, 1H), 6.02 (s, 1H), 4.86 (s, 2H), 4.18 (m, 1H), 3.98 (m, 2H), 3.74 (m, 1H). MS (M+1): 562.3

### Assay

A useful assay to determine the DGAT inhibitory activity of the inventive compounds is described below:

The in vitro assay to identify DGAT1 inhibitors uses human DGAT1 enzyme expressed in Sf9 insect cells prepared as microsomes. The reaction is initiated by the addition of the combined substrates 1,2-dioleoyl-sn-glycerol and [¹⁴C]-palmitoyl-Co A and incubated with test compounds and microsomal membranes for 2 hours at room temperature. The assay is stopped by adding 0.5 mg wheat germ agglutinin beads in assay buffer with 1% Brij-35 and 1% 3-cholamidopropyldimethyl-ammonio-1-propane sulfonate. Plates are sealed with TopSeal and incubated for 18 hours to allow the radioactive triglyceride product to come into proximity with the bead. Plates are read on a TopCount instrument.

Percent inhibition was calculated as the percent of (test compound inhibition minus non-specific binding) relative to (total binding minus non-specific binding). IC₅₀ values were determined by curve fitting the data to a Sigmoidal dose-response in GraphPad Prism utilizing the following equation:

Y=A+(B−A)/(1+10̂((Log IC₅₀−X))),

where A and B are the bottom and top of the curve (highest and lowest inhibition), respectively, and X is the logarithm of concentration. Some compounds and their 1050 values are shown below:
A represents IC50=0-10 nM
B represents IC50=11-100 nM
C represents IC50=101-500 nM

| | | hDGAT |
|---|---|---|
| Compound | Structure | IC50 (nM) |
| 78 | 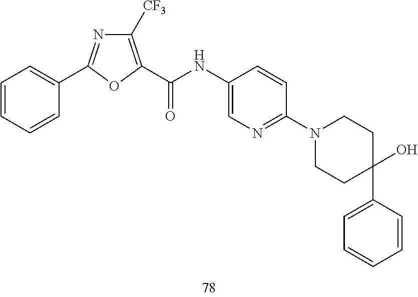 | C |
| 82 | 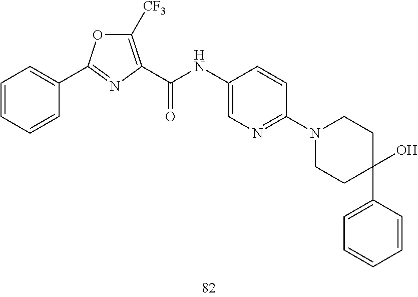 | C |
| 62 | 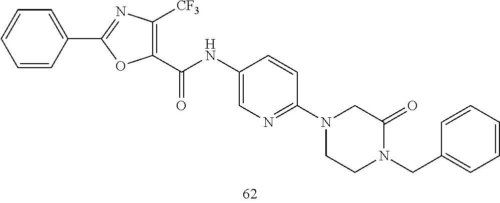 | B |
| 72 | 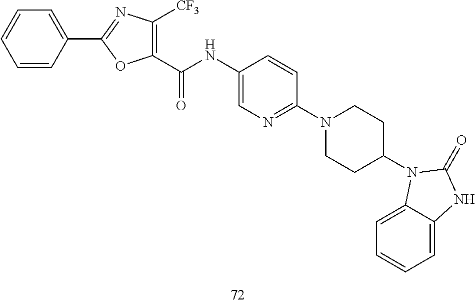 | C |
| 151 | 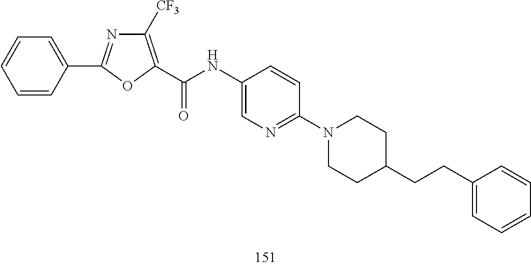 | C |
| 153 | 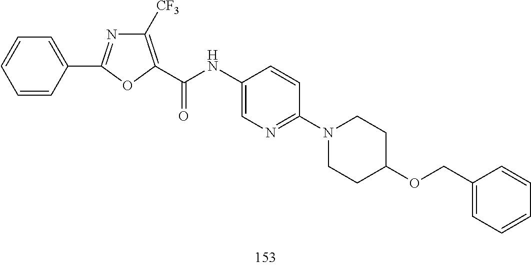 | B |
| 155 | 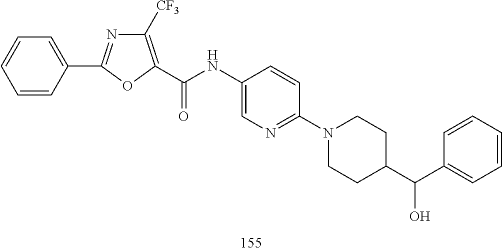 | B |
| 157 | 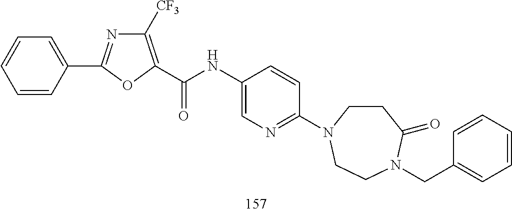 | C |
| 164 | 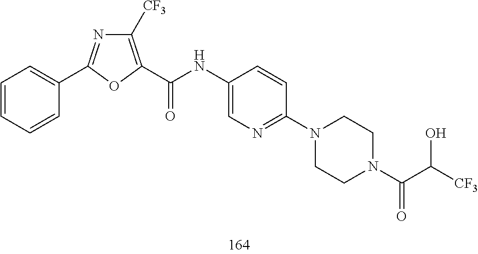 | B |
| 165 | 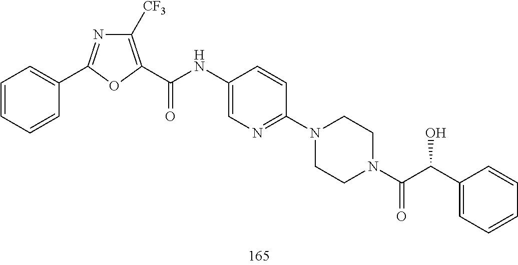 | B |
| 168 | 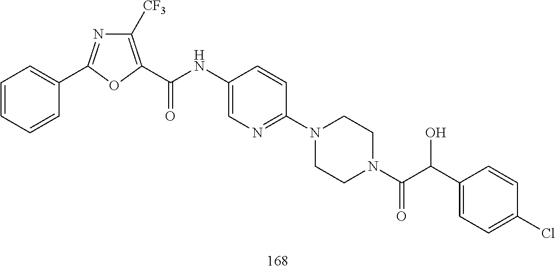 | B |
| 169 | 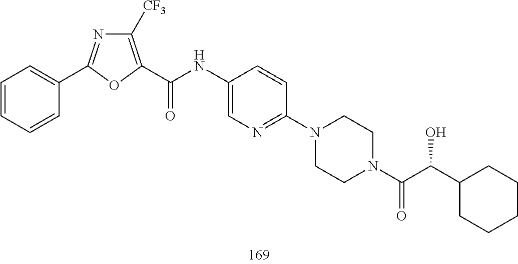 | B |
| 170 | 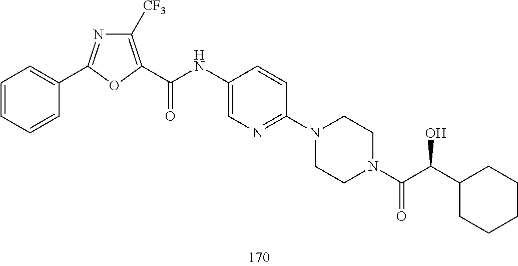 | B |
| 171 | 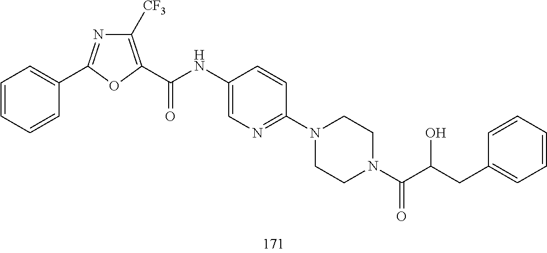 | B |
| 172 | 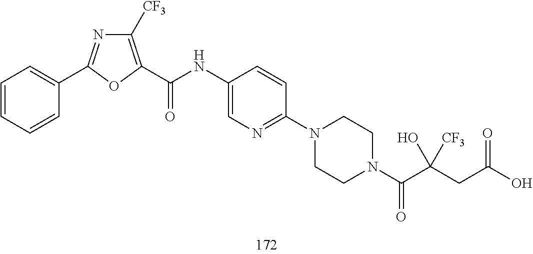 | B |
| 176 | 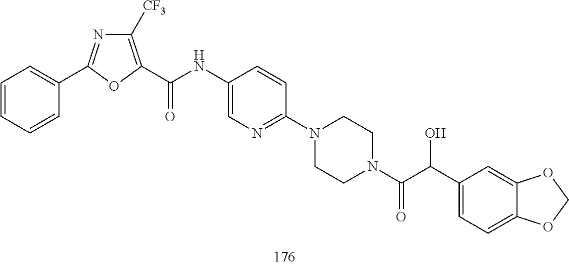 | B |
| 179 | 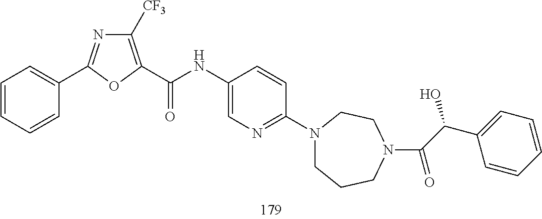 | B |
| 180 | 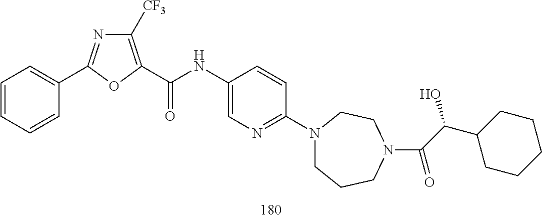 | B |
| 181 | 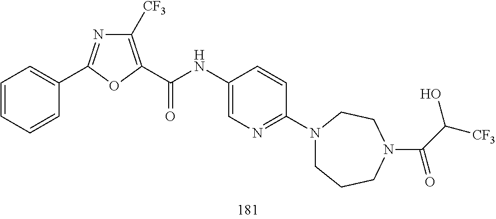 | B |
| 182 | 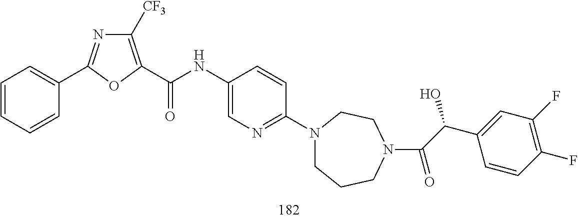 | B |

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

What is claimed is:

1. A compound, or a pharmaceutically acceptable salt thereof, the compound being represented by the general formula I: 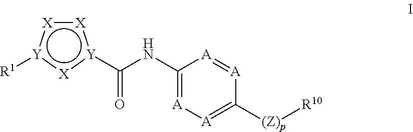
wherein:
each A is independently selected from C(R³) and N;
or alternately the moiety: 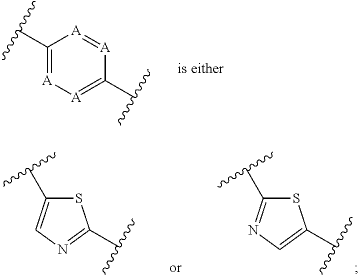
X is independently selected from C(R³), N, N(R⁴), O and S, provided that no more than one X is S or O, and at least one X or one Y is N, O, or S;
Y is independently selected from C and N;
Z is independently a bond, O or NR⁴;
p is 0 or 1;
R¹ is selected from aryl, heteroaryl, alkyl or cycloalkyl, wherein said aryl is unsubstituted or optionally independently substituted with one or more moieties which are the same or different, each substituent being independently selected from the group consisting of alkyl, haloalkoxy, methoxy-ethoxy alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —ON, —OR^{c}, —C(O)R^{c}, —C(O)OR^{c}, —C(O)N(R^{c})(R^{d}), —SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}), —N(R^{c})(R^{d}), -alkyl-N(R^{c})(R^{d}), —N(R^{c})C(O)R^{d}, —CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d}, —N(R^{c})S(O)₂N(R^{d})(R^{b}), —N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{c}, —N₃, —NO₂ and —S(O)₂R^{c}, wherein each R^{b}, R^{c} and R^{d}, is independently selected;
R³ is selected from the group of H, lower alkyl, hydroxy, halo, O-alkyl, O-haloalkyl, O-cycloalkyl, S-alkyl, S-haloalkyl, CN, CF₃, —SF₅, —OSF₅, —Si(R^{c})₃, —SR^{c}, cycloalkyl, heterocyclyl, haloalkyl, aryl, heteroaryl, N-alkyl, N-haloalkyl, NH₂, and N-cycloalkyl;
R⁴ is selected from the group of H, lower alkyl, cycloalkyl, heterocyclyl, haloalkyl, aryl, and heteroaryl;
R¹⁰ is either (i) a 4-8 membered heterocyclyl ring having from 1 to 3 ring N atoms, or (ii) a bicyclic heterocyclyl ring having from 1 to 3 ring N atoms,
wherein each of said heterocyclyl ring or bicyclic heterocyclyl ring for R¹⁰ is optionally fused with a heteroaryl ring, further wherein each of said heterocyclyl ring or bicyclic heterocyclyl ring for R¹⁰ is independently unsubstituted or optionally substituted, off of either (i) a ring N atom or (ii) a ring carbon atom on said heterocyclyl ring or said bicyclic heterocyclyl ring, with one or more G moieties wherein said G moieties can be the same or different, each G moiety being independently selected from the group consisting of: 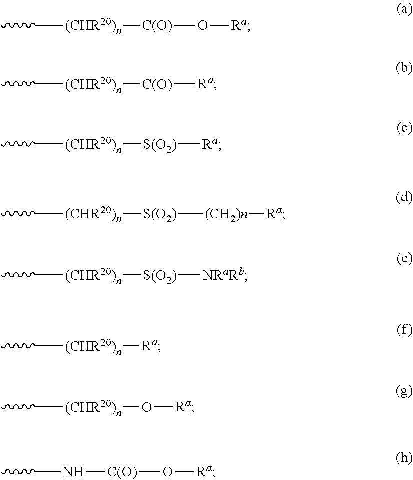
off of only C and not off of N, with the proviso that R¹⁰ is not a 5- or 6-membered heterocyclyl ring; 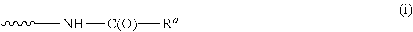
off of only C and not off of N, with the proviso that R¹⁰ is not a 5- or 6-membered heterocyclyl ring; 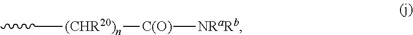
with the proviso that R¹⁰ is not a 5- or 6-membered heterocyclyl ring; 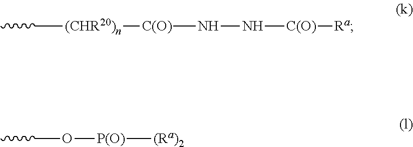
off of only C and not off of N; 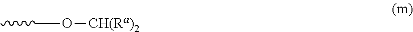
off of only C and not off of N; (n) an oxo group off of only C and not off of N; 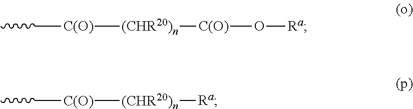
and (q) a spirocyclyl group;
wherein R^{a} is selected from the group consisting of hydrogen, hydroxy, CN, halo, alkyl, haloalkyl, alkenyl, alkynyl, aryl, heteroaryl, heterocyclyl, cycloalkyl or spirocyclyl, wherein each of said alkyl, alkenyl, alkynyl, aryl, heteroaryl, heterocyclyl and cycloalkyl is unsubstituted or optionally independently substituted with one or more moieties which are the same or different, each moiety being selected independently from the group consisting of O-haloalkyl, S-haloalkyl, CN, NO₂, CF₃, cycloalkyl, heterocyclyl, haloalkyl, aryl, heteroaryl, N-alkyl, N-haloalkyl, and N-cycloalkyl; alkyl, alkenyl, alkynyl, cycloalkylalkyl, cycloalkenyl, heterocyclylalkyl, aryl, arylalkyl, heteroaryl, heteroarylalkyl, halo, —OR^{c}, —C(O)R^{c}, —C(O)OR^{c}, —C(O)N(R^{c})(R^{d}), SF₅, —OSF₅, —Si(R^{c})₃, —SR^{c}, —S(O)N(R^{c})(R^{d}), —CH(R^{c})(R^{d}), —S(O)₂N(R^{c})(R^{d}), —C(═NOR^{c})R^{d}, —P(O)(OR^{c})(OR^{d}), —N(R^{c})(R^{d}), -alkyl-N(R^{c})(R^{d}), —N(R^{c})C(O)R^{d}, —CH₂—N(R^{c})C(O)R^{d}, —CH₂—N(R^{c})C(O)N(R^{d})(R^{b}), —CH₂—R^{c}; —CH₂N(R^{c})(R^{d}), —N(R^{c})S(O)R^{d}, —N(R^{c})S(O)₂R^{d}, —CH₂—N(R^{c})S(O)₂R^{d}, —N(R^{c})S(O)₂N(R^{d})(R^{b}), —N(R^{c})S(O)N(R^{d})(R^{b}), —N(R^{c})C(O)N(R^{d})(R^{b}), —CH₂—N(R^{c})C(O)N(R^{d})(R^{b}), —N(R^{c})C(O)OR^{d}, —CH₂—N(R^{c})C(O)OR^{d}, —S(O)R^{c}, ═NOR^{c}, —N₃, and —S(O)₂R^{c}; and
wherein each R^{b}, R^{c} and R^{d} is independently selected;
R^{b} is H, lower alkyl, cycloalkyl, aryl, heteroaryl or heterocycloalkyl;
R^{c} is H, lower alkyl, cycloalkyl, aryl, heteroaryl or heterocycloalkyl;
R^{d} is H, lower alkyl, cycloalkyl, aryl, heteroaryl or heterocycloalkyl;
wherein each of said alkyl, cycloalkyl, aryl, heteroaryl or heterocycloalkyl in R^{b}, R^{c}, and R^{d} can be unsubstituted or optionally independently substituted with 1-2 substituents independently selected from halo, OH, NH₂, CF₃, CN, Oalkyl, NHalkyl, N(alkyl)₂ and Si(alkyl)₃;
R²⁰ is H, —OH, halo, or —CF₃;
m is 1-3, and
n is 0-3.

2. A compound, or a pharmaceutically acceptable salt thereof, wherein the compound is selected from the group consisting of the following:

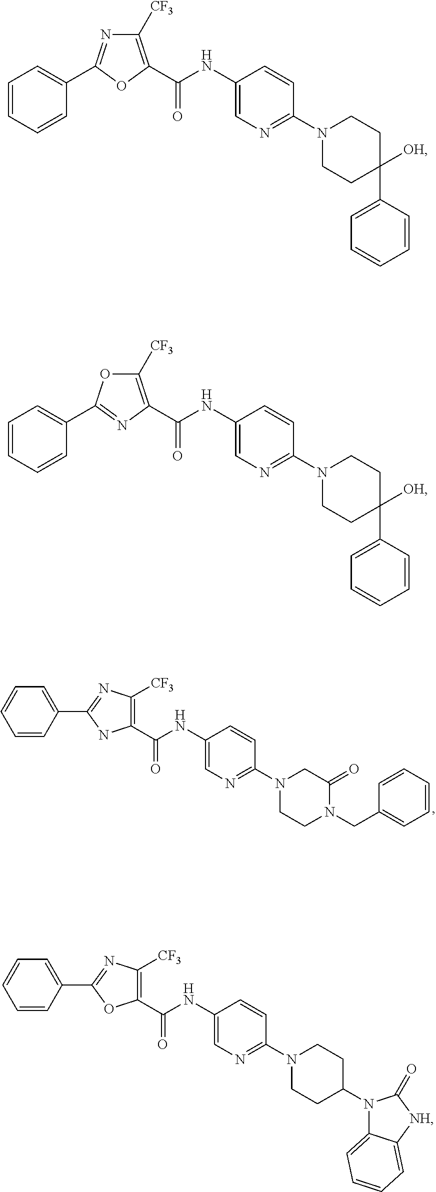

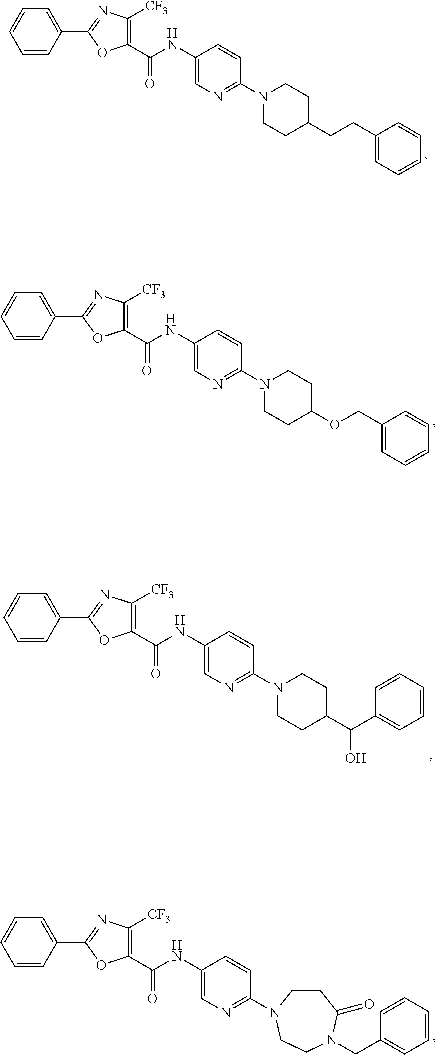

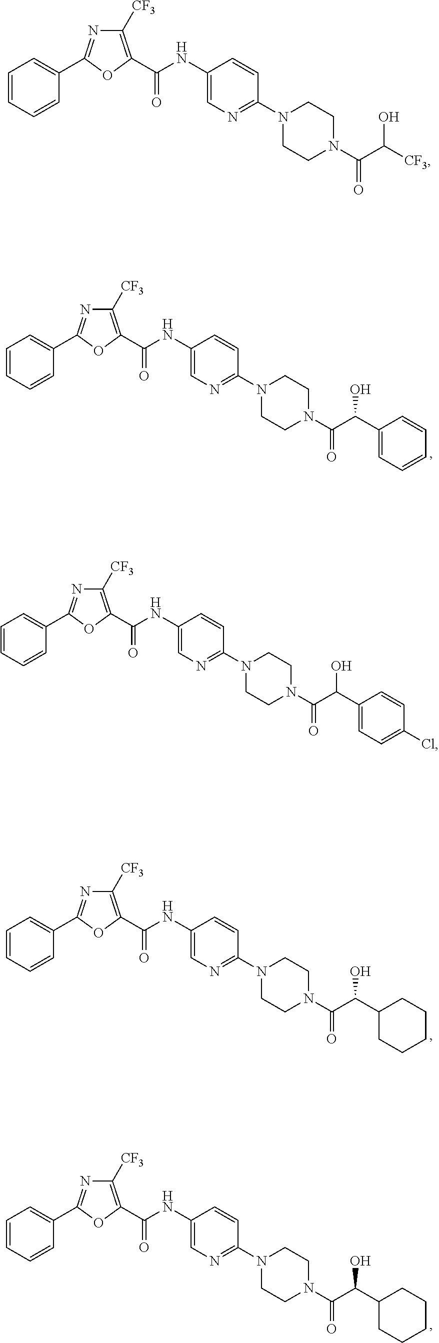

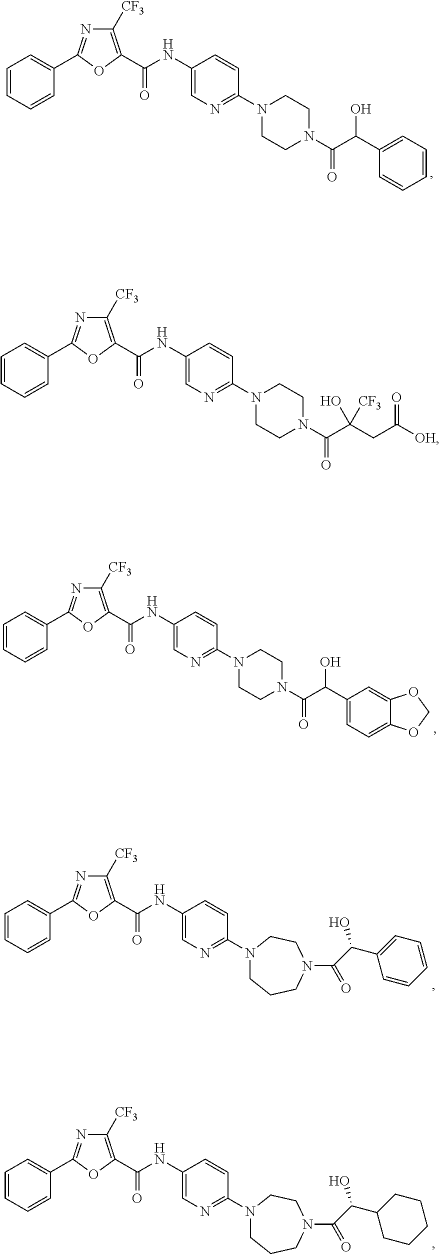

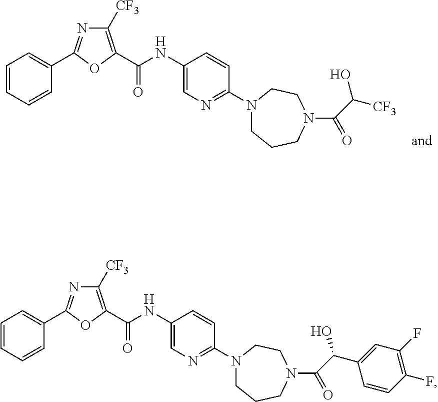

3. A pharmaceutical composition comprising an effective amount of at least one compound of claim 1, or a pharmaceutically acceptable salt thereof, and a pharmaceutically acceptable carrier.

4. A pharmaceutical composition comprising an effective amount of at least one compound of claim 2, or a pharmaceutically acceptable salt thereof, and a pharmaceutically acceptable carrier.

5. A method of treating a cardiovascular disease, a metabolic disorder, obesity, an obesity-related disorder, dyslipidemia, diabetes, a diabetic complication, impaired glucose tolerance or impaired fasting glucose in a patient, comprising administering to the patient an effective amount of at least one compound of claim 1, or a pharmaceutically acceptable salt thereof.

6. A method of treating a cardiovascular disease, a metabolic disorder, obesity, an obesity-related disorder, dyslipidemia, diabetes, a diabetic complication, impaired glucose tolerance or impaired fasting glucose in a patient, comprising administering to the patient an effective amount of at least one compound of claim 2, or a pharmaceutically acceptable salt thereof.

7. The method of claim 5, wherein the disease treated is diabetes.

8. The method of claim 6, wherein the diabetes is type II diabetes.

9. The method of claim 5, wherein the disease treated is obesity.

10. The method of claim 5, wherein the disease treated is a metabolic disorder.

11. The method of claim 5, further comprising administering to the patient an effective amount of at least one additional therapeutic agent, wherein the additional therapeutic agent(s) is selected from an antidiabetic agent or an antiobesity agent.

12. The method of claim 11, wherein the disease treated is diabetes.

13. The method of claim 12, wherein the diabetes is type II diabetes.

14. The method of claim 6, wherein the disease treated is a metabolic disorder.

15. The method of claim 6, further comprising administering to the patient an effective amount of at least one additional therapeutic agent, wherein the additional therapeutic agent(s) is selected from an antidiabetic agent or an antiobesity agent.
